(19) United States
(12) Patent Application Publication
(10) Pub. No.: US 2012/0328602 A1
(43) Pub. Date: Dec. 27, 2012

(54) COMPOSITIONS AND METHODS FOR TREATING CONDITIONS ASSOCIATED WITH CERAMIDE BIOSYNTHESIS

(75) Inventor: Daniela Salvemini, Chesterfield, MO (US)

(73) Assignee: SAINT LOUIS UNIVERSITY, St. Louis, MO (US)

(21) Appl. No.: 13/586,675

(22) Filed: Aug. 15, 2012

Related U.S. Application Data
(62) Division of application No. 12/565,634, filed on Sep. 23, 2009.
Application No. 12/565,634 is a continuation-in-part of application No. 11/695,519, filed on Apr. 2, 2007, now abandoned.

(30) Foreign Application Priority Data
Apr. 2, 2007 (US) PCT/US2008/059123

(51) Int. Cl. A61K 31/485; A61P 25/36; A61K 39/395; A61K 31/713; A61K 31/7105

(52) U.S. Cl. 424/130.1; 514/44 A; 514/44 R; 514/282

(57) ABSTRACT

Provided are a pharmaceutical composition and a method for reducing, preventing, or delaying the development of a biological condition associated with administration of an opioid drug, in particular, tolerance to and/or physical dependence on an opioid drug. The pharmaceutical composition includes an opioid drug, a ceramide biosynthesis inhibitor and a pharmaceutically acceptable carrier. The method of treatment involves administration of an opioid drug and a ceramide biosynthesis inhibitor. Also provided are a method of screening for an agent that reduces, prevents or delays the development of tolerance to and/or physical dependence on an opioid drug as well as compositions comprising a dsRNA for inhibiting ceramide biosynthesis in a cell and a vector for expressing a shRNA for inhibiting ceramide biosynthesis in a cell.

### CROSS-REFERENCE TO RELATED APPLICATIONS

The present application is a divisional of U.S. patent application Ser. No. 12/565,634 entitled “COMPOSITIONS AND METHODS FOR TREATING CONDITIONS ASSOCIATED WITH CERAMIDE BIOSYNTHESIS” filed on Sep. 23, 2009 which is a continuation-in-part of U.S. patent application Ser. No. 11/695,519 entitled “INHIBITORS OF THE CERAMIDE METABOLIC PATHWAY AS ADJUNCTS TO OPIATES FOR PAIN” filed on Apr. 2, 2007, which is now abandoned, with the United States Patent and Trademark Office, the contents of which are hereby incorporated by reference in their entirety to the extent permitted by law.

### STATEMENT REGARDING FEDERALLY SPONSORED RESEARCH OR DEVELOPMENT

Not applicable.

### INCORPORATION-BY-REFERENCE OF SEQUENCE LISTING

The Sequence Listing, which is a part of the present disclosure, includes a computer readable file “5015227-5_ST25.TXT” generated by U.S. Patent & Trademark Office Patent In version 3.5 software comprising nucleotide and/or amino acid sequences of the present invention. The subject matter of the Sequence Listing is incorporated herein by reference in its entirety.

### FIELD

The present teachings relate to methods and compositions for treating opioid tolerance in a subject.

### INTRODUCTION

Chronic, severe pain is a significant health problem both in the U.S. and worldwide. One third of Americans suffer from some form of chronic pain, and in more than thirty percent of these cases the pain becomes resistant to analgesic therapy. The economic impact of pain in the U.S. is approximately $100 billion annually (Renfrey et al., 2003).

Opiate analgesics, typified by morphine sulfate, are the most effective treatments for acute and chronic severe pain. The clinical utility of opiates is, however, hampered by the development of analgesic tolerance, which necessitates the use of escalating doses to achieve an equivalent level of pain relief (Foley, 1995).

Adaptive modifications in cellular responsiveness and, in particular, desensitization and down-regulation of opioid receptors are thought to be at the root of opioid tolerance (Taylor et al., 2001). An alternative hypothesis, however, is that the stimulation of opioid receptors over time triggers activation of anti-opioid systems that, in turn, reduce sensory thresholds, thereby resulting in hypersensitivity to tactile stimulation (i.e. Allodynia) and to noxious thermal stimulation (i.e. hyperalgesia). As a corollary to this hypothesis, such opioid-induced hypersensitivity paradoxically diminishes the net analgesic effect of the opioid agonists (Ossipov et al., 2004; Simonnet et al., 2003; Rothman, 1992). Support for this alternative hypothesis has been evidenced in vivo in animals (Mao et al., 1995; Celerier et al., 2000; Celerier et al., 2001) and in human subjects (Amer et al., 1988; De Conno et al., 1991; Devulder, 1997). It is thought, therefore, that analgesic tolerance arises when pain facilitatory systems become sensitized or hyperactive after repeated opioid use. In other words, hyperalgesia and antinociceptive/analgesic tolerance are a result of the same disorder stemming from opiate use.

Ceramide is a sphingolipid signaling molecule that is generated from de novo synthesis mediated by serine palmitoyltransferase (SPT) and ceramide synthase (CeS), as well as by enzymatic hydrolysis of sphingomyelin by sphingomyelinases (SMases). The de novo pathway is stimulated by numerous chemotherapeutics and usually results in prolonged ceramide elevation. Ultimately, the steady-state availability of ceramide is regulated by ceramidases that convert ceramide to sphingosine by catalyzing the hydrolysis of the ceramide amide group. One form of acid ceramidease may also be a secreted enzyme, whereas a form of neutral ceramidase may be mitochondrial and hence may affect ceramide synthase-mediated ceramide signaling in that cellular compartment.

Ceramide is also generated by enzymatic hydrolysis of sphingomyelin by sphingomyelinases. Sphingomyelin is generated by the enzyme sphingomyelin synthase (SMS) and is localized to the outer leaflet of the plasma membrane, providing a semi-permeable barrier to the extracellular environment (Tafesse et al., 2006). Several isoforms of sphingomyelinase can be distinguished by pH optima for their activity, and these are referred to as acid (ASMase), neutral (NSMase), or alkaline SMase. Of these isoforms, NSMase and ASMase are rapidly activated by diverse stressors and cause increased ceramide levels within minutes to hours. Mammalian ASMase and NSMase have been cloned from distinct genes (Horinouchi et al., 1995), ASMase was originally described as a lysosomal enzyme (pH optimum 4.5-5) that is defective in patients with Niemann-Pick disease. More recently, a secretory isoform was identified that targets the plasma membrane and is secreted extracellularly (Schissel et al., 1998; Schissel et al., 1996). The lysosomal and secretory ASMase are derived from the same inactive 75 kDa precursor, but differ by their NH₂-termini and display different glycosylation patterns that likely determine their targeting. Secretory ASMase hydrolyzes cell surface sphingomyelin to initiate signaling, whereas neutral SMase is primarily located to the plasma membrane. Consequently, each SMase generates separate intracellular pools of ceramide.

Opioid tolerance as described above has not been known to be related to ceramide levels and ceramide biosynthesis prior the work reported herein.

Other conditions include those related to peroxynitrite which is an anion having the formula ONOO⁻. The molecule is an oxidant and nitrating agent that can damage a wide array of biological molecules, including DNA and proteins. Peroxynitrite reacts nucleophilically with carbon dioxide. The concentration of carbon dioxide in vivo is about 1 mM, and its reaction with peroxynitrite occurs quickly. Free radicals associated with this reaction are believed to be responsible for conditions involving peroxynitrite-related cellular damage. These conditions have also not been known to be related to ceramide levels and ceramide biosynthesis prior to the present work.

### SUMMARY

Accordingly, the present invention provides pharmaceutical compositions and methods for treating, preventing, or inhibiting biological conditions associated with ceramide biosynthesis.

Thus, the present invention provides, in various embodiments, a pharmaceutical composition that is suitable for treating, preventing or inhibiting a biological condition associated with ceramide biosynthesis accompanying administration of an opioid drug. The pharmaceutical composition includes an analgesic amount of an opioid drug, a therapeutically effective amount of a ceramide biosynthesis inhibitor and a pharmaceutically acceptable carrier. In various aspects of this embodiment, the opioid drug may any opioid drug and, in particular, one that targets one or more of μ-opioid receptors, δ-opioid receptors or κ-opioid receptors. In various embodiments, the opioid drug may be morphine. The ceramide biosynthesis inhibitor may be an inhibitor of any one or more ceramide biosynthetic enzymes. Such enzymes may include a sphingomyelinase, a serine palmitoyltransferase, a 3-ketosphinganine reductase, ceramide synthase, a dihydroceramide desaturase or any combination thereof. In particular, the ceramide biosynthesis inhibitor may be Fumonisin B1 (FB1), tyclodecan-9-xanthogenate (D609), myriocin or any combination thereof.

In various other embodiments, the present invention includes a method for reducing, preventing or delaying the development of tolerance to, and/or physical dependence on, an opioid drug that targets an opioid receptor. The method includes administering to a subject in need thereof, an analgesic amount of the opioid drug and a therapeutically effective amount of an agent that inhibits ceramide biosynthesis inhibitor. The ceramide synthesis inhibitor may be administered within a therapeutically effective time with respect to administering the opioid drug. In various aspects of this embodiment, the ceramide synthesis inhibitor may be administered prior to administration of the opioid drug, for example about 15 minutes, about 2 hours, or about 24 hours prior to administration of the opioid drug; the ceramide synthesis inhibitor may be administered at substantially the same time as the opioid drug; or the ceramide synthesis inhibitor may be administered after administration of the opioid drug, for example about 15 minutes, about 2 hours, or about 24 hours after administration of the opioid drug. The opioid drug may be any opioid drug and, in particular, one that targets one or more of μ-opioid receptors, δ-opioid receptors or κ-opioid receptors. In various embodiments, the opioid drug may be morphine. The agent that inhibits ceramide biosynthesis may be an inhibitor of any one or more ceramide biosynthetic enzymes in which the ceramide biosynthetic enzyme may be a sphingomyelinase, a serine palmitoyltransferase, a 3-ketosphinganine reductase, a ceramide synthase or a dihydroceramide desaturase. In particular, the ceramide biosynthesis inhibitor may be Fumonisin B1 (FB1), tyclodecan-9-xanthogenate (D609), myriocin or any combination thereof.

The present invention also includes, in various embodiments, a method of screening for an agent that reduces, prevents or delays the development of tolerance to, and/or physical dependence on, an opioid drug that targets an opioid receptor. The method includes (a) contacting a cell comprising the opioid receptor, with an opioid drug; (b) contacting the cell with a test agent; (c) determining whether the test agent inhibits biosynthesis of ceramide in the presence of the opioid drug and/or reduces or prevents an increase in ceramide elicited by the opioid drug; and (d) selecting the test agent as an agent that may reduce, prevent or delay the development of tolerance to and/or physical dependence on the opioid drug if the test agent inhibits biosynthesis of ceramide and/or reduces or prevents an increase in ceramide levels elicited by the opioid drug. The opioid drug may any opioid drug and, in particular, one that targets one or more of μ-opioid receptors, δ-opioid receptors or κ-opioid receptors. In various embodiments, the opioid drug may be morphine. The agent that inhibits ceramide biosynthesis may be an inhibitor of any one or more ceramide biosynthetic enzymes in which the ceramide biosynthetic enzyme may be a sphingomyelinase, a serine palmitoyltransferase, a 3-ketosphinganine reductase, a ceramide synthase or a dihydroceramide desaturase. Both in vitro and in vivo screening methods are within the scope of the present invention.

In various other embodiments, the present invention also includes a method for treating a biological condition associated with ceramide biosynthesis accompanying administration of an opioid in a subject. The method includes administering to a subject receiving administration of the opioid drug and having the biological condition, a therapeutically effective amount of an agent that inhibits ceramide biosynthesis. In various embodiments, the biological condition may be opioid tolerance, nitroxidative stress or neuroimmune activation. The ceramide synthesis inhibitor may be administered within a therapeutically effective time with respect to administering the opioid drug. In various aspects of this embodiment, the ceramide synthesis inhibitor may be administered prior to administration of the opioid drug, for example about 15 minutes, about 2 hours, or about 24 hours prior to administration of the opioid drug; the ceramide synthesis inhibitor may be administered at substantially the same time as the opioid drug; or the ceramide synthesis inhibitor may be administered after administration of the opioid drug, for example about 15 minutes, about 2 hours, or about 2.4 hours after administration of the opioid drug. In various aspects of this embodiment, the opioid drug may any opioid drug and, in particular, one that targets one or more of μ-opioid receptors, δ-opioid receptors or κ-opioid receptors. In various embodiments, the opioid drug may be morphine. In various embodiments, the agent that inhibits ceramide biosynthesis may be an inhibitor of any one or more ceramide biosynthetic enzymes in which the ceramide biosynthetic enzyme may be a sphingomyelinase, a serine palmitoyltransferase, a 3-ketosphinganine reductase, a ceramide synthase or a dihydroceramide desaturase. In particular, the ceramide biosynthesis inhibitor may be Fumonisin B1 (FB1), tyclodecan-9-xanthogenate (D609), myriocin or any combination thereof.

The present invention also includes, in various embodiments, a dsRNA for inhibiting ceramide biosynthesis in a cell. The dsRNA includes a sense strand and an antisense strand in which the sense strand is substantially complementary to the antisense strand. Further, the antisense strand includes a region of complementarity having a sequence substantially complementary to a target sequence of an RNA encoding a ceramide biosynthesis enzyme. The target sequence may be not more than about 30 contiguous nucleotides in length. Upon contact with a cell comprising the target sequence, the dsRNA inhibits ceramide biosynthesis. In various embodiments, the enzyme encoded by the RNA containing the target sequence, may be a sphingomyelinase, a serine palmitoyltransferase, a 3-ketosphinganine reductase, a ceramide synthase or a dihydroceramide desaturase. In various embodiments, the antisense strand may include a region of complementarity having a sequence substantially complementary to a target sequence of not more than about 30 contiguous and, in particular, from about 19 to about 21 contiguous nucleotides of a sequence encoding SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33 or SEQ ID NO: 35. In various embodiments, the antisense strand may include a region of complementarity having a sequence substantially complementary to a target sequence of not more than about 30 contiguous nucleotides and, in particular, from about 119 to about 21 contiguous nucleotides of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34 or SEQ ID NO: 36.

In still other embodiments, the present invention includes a vector for expressing shRNA for inhibiting ceramide biosynthesis in a cell. The vector includes a sense strand, a hairpin linker, and an antisense strand in which the sense strand is substantially complementary to the antisense strand. Further, the sense strand includes a region of complementarity having a sequence substantially complementary to a target sequence of an RNA encoding a ceramide biosynthesis enzyme. The target sequence may be not more than about 30 contiguous nucleotides in length. Upon contact with a cell comprising the target sequence, the shRNA inhibits ceramide biosynthesis. In various embodiments, the enzyme encoded by the RNA containing the target sequence, may be a sphingomyelinase, a serine palmitoyltransferase, a 3-ketosphinganine reductase, a ceramide synthase or a dihydroceramide desaturase. In various embodiments, the sense strand may include a region of complementarity having a sequence substantially complementary to a target sequence of not more than about 30 contiguous and, in particular, from about 19 to about 2.1 contiguous nucleotides of a sequence encoding SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33 or SEQ ID NO: 35. In various embodiments, the sense strand may include a region of complementarity having a sequence substantially complementary to a target sequence of not more than about 30 contiguous nucleotides and, in particular, from about 19 to about 21 contiguous nucleotides of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34 or SEQ ID NO: 36.

The present invention also includes, in various embodiments, a pharmaceutical composition tier inhibiting ceramide biosynthesis in a cell in which the pharmaceutical composition includes a dsRNA that inhibits ceramide biosynthesis and a pharmaceutically acceptable carrier. The dsRNA includes a sense strand and an antisense strand in which the sense strand is substantially complementary to the antisense strand. Further, the antisense strand includes a region of complementarily having a sequence substantially complementary to a target sequence of an RNA encoding a ceramide biosynthesis enzyme. The target sequence may be not more than about 30 contiguous nucleotides in length. Upon contact with a cell comprising the target sequence, the dsRNA inhibits ceramide biosynthesis. In various embodiments, the enzyme encoded by the RNA containing the target sequence, may be a sphingomyelinase, a serine palmitoyltransferase, a 3-ketosphinganine reductase, a ceramide synthase or a dihydroceramide desaturase. In various embodiments, the antisense strand may include a region of complementarity having a sequence substantially complementary to a target sequence of not more than about 30 contiguous and, in particular, from about 19 to about 2.1 contiguous nucleotides of a sequence encoding SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33 or SEQ ID NO: 35. In various embodiments, the antisense strand may include a region of complementarity having a sequence substantially complementary to a target sequence of not more than about 30 contiguous nucleotides and, in particular, from about 19 to about 21 contiguous nucleotides of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34 or SEQ ID NO: 36.

The present invention also includes, in various embodiments, a pharmaceutical composition for inhibiting ceramide biosynthesis in a cell in which the pharmaceutical composition includes a vector for expressing a shRNA that inhibits ceramide biosynthesis and a pharmaceutically acceptable carrier. The vector includes a sense strand, a hairpin linker, and an antisense strand in which the sense strand is substantially complementary to the antisense strand. Further, the sense strand includes a region of complementarity having a sequence substantially complementary to a target sequence of an RNA encoding a ceramide biosynthesis enzyme. The target sequence may be not more than about 30 contiguous nucleotides in length. Upon contact with a cell comprising the target sequence, the shRNA inhibits ceramide biosynthesis. In various embodiments, the enzyme encoded by the RNA containing the target sequence, may be a sphingomyelinase, a serine palmitoyltransferase, a 3-ketosphinganine reductase, a ceramide synthase or a dihydroceramide desaturase. In various embodiments, the sense strand may include a region of complementarity having a sequence substantially complementary to a target sequence of not more than about 30 contiguous and, in particular, from about 19 to about 21 contiguous nucleotides of a sequence encoding SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33 or SEQ ID NO: 35. In various embodiments, the sense strand may include a region of complementarily having a sequence substantially complementary to a target sequence of not more than about 30 contiguous nucleotides and, in particular, from about 19 to about 21 contiguous nucleotides of SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34 or SEQ ID NO: 36.

The present invention also includes, in various embodiments, a method for treating a biological condition associated with a compound downstream of ceramide in a metabolic pathway that includes ceramide, in a subject. The method includes administering to a subject in need thereof a therapeutically effective amount of an agent that inhibits ceramide biosynthesis. In various embodiments, the compound downstream of ceramide may be peroxynitrite, a cytokine such as TNF-α, IL-1β, of IL-6, transcription factor NK-κB, manganese superoxide dismutase or a combination thereof. The agent that inhibits ceramide biosynthesis targets at least one ceramide-biosynthetic enzyme such as, for example, a sphingomyelinase, a serine palmitoyltransferase, a 3-ketosphinganine reductase, a ceramide synthase, adihydroceramide desaturase or any combination thereof. In particular, the ceramide biosynthesis inhibitor may be FB1, D609, myriocin or any combinations thereof.

### DRAWINGS

Those of skill in the art will understand that the drawings, described below, are for illustrative purposes only. The drawings are not intended to limit the scope of the present teachings in any way.

FIG. 1. Schematic illustration of the ceramide metabolic pathways.

FIG. 2, Graph illustrating the inhibition of antinociceptive tolerance in mice by inhibition of ceramide synthesis using Fumonisin B (FB1), tyclodecan-9-xanthogenate (D609), and myriocin.

FIG. 3. Series of photomicrographs that illustrate the reduction (as compared to control mice) of ceramide in the spinal column of mice after treatment with morphine and ceramide synthesis inhibitor FB1.

FIG. 4. Graph illustrating that co-administration of FB1 with morphine blocks an increase in ceramide levels that occurs in control mice given morphine alone.

FIG. 5, Series of graphs demonstrating that ceramide inhibitors effectively prevent the development of morphine antinociceptive tolerance.

FIG. 6. Series of photographs depicting immunohistochemical detection of ceramide.

FIG. 7. Graph illustrating the lack of effect of ceramide inhibitors on antinociceptive responses to acute morphine in non-tolerant animals.

FIG. 8. Series of photographs and a graph demonstrating that the development of nitroxidative stress during morphine antinociceptive tolerance is blocked by an inhibitor of ceramide biosynthesis, namely FB1.

FIG. 9. Series of photographs and graphs demonstrating that NF-κB activation during morphine antinociceptive tolerance is blocked by an inhibitor of ceramide biosynthesis, namely FB1.

FIG. 10. Series of photographs illustrating that ceramide preferentially co-localizes with glial cells but not with neurons.

FIG. 11. Series of photographs illustrating that activation of spinal glial cells during morphine antinociceptive tolerance is blocked by an inhibitor of ceramide biosynthesis, namely FB1.

FIG. 12. Series of graphs illustrating that increased spinal production of proinflammatory and pronociceptive cytokines is blocked by an inhibitor of ceramide biosynthesis, namely FB1.

FIG. 13. Diagram illustrating certain findings associated with the present invention.

### DETAILED DESCRIPTION

### Abbreviations and Definitions

To facilitate understanding of the invention, a number of terms and abbreviations as used herein are defined below as follows:

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A atone, B alone, C atone, A and B in combination, A and C in combination, B and C combination or A, B, and C in combination.

Agent or Therapeutic Agent: As used herein, the terms “agent” and “therapeutic agent” refer to any natural or synthesized composition that when administered to a subject relieves the subject of disease or improves health. More specifically, as referred to herein, agents and therapeutic agents include chemical compounds, polypeptides, amino acids, oligonucleotides or combinations thereof. In particular, the term “agent” may refer to a substance that inhibits ceramide biosynthesis and/or reduces ceramide levels in a subject.

Antisense Strand: The term “antisense strand” refers to the strand of a dsRNA which includes a region that is substantially complementary to a target sequence. As used herein, the term “region of complementarity” refers to the region on the antisense strand that is substantially complementary to a sequence, for example a target sequence, as defined herein. Where the region of complementarity is not fully complementary to the target sequence, the mismatches are most tolerated in the terminal regions and, if present, are generally in a terminal region or regions, e.g., within 6, 5, 4, 3, or 2 nucleotides of the 5′ and/or 3′ terminus. In certain aspects of the invention, the mismatches can be located within 6, 5, 4, 3, or 2 nucleotides of the 5′ terminus of the antisense strand and/or the 3′ terminus of the sense strand.

Bind, Binds or Interacts With: As used herein, “bind,” “binds,” or “interacts with” means that one molecule recognizes and adheres to a particular second molecule in a sample, but does not substantially recognize or adhere to other structurally unrelated molecules in the sample. Generally, a first molecule that “specifically binds” a second molecule has a binding affinity greater than about 10⁵ to 10⁶ moles/liter for that second molecule.

Complementary: As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions, e.g., stringent conditions, with an oligonucleotide polynucleotide comprising the second nucleotide sequence, as will be understood by the skilled person. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides. This includes base-pairing of the oligonucleotide or polynucleotide comprising the first nucleotide sequence to the oligonucleotide or polynucleotide comprising the second nucleotide sequence over the entire length of the first and second nucleotide sequence. Such sequences can be related to as “fully complementary” with respect to each other herein. However, where a first sequence is referred to as “substantially complementary” with respect to a second sequence herein, the two sequences can be fully complementary, or they may form one or more, but generally not more than 4, 3 or 2 mismatched base pairs upon hybridization, while retaining the ability to hybridize under the conditions most relevant to their ultimate application. However, where two oligonucleotides are designed to form, upon hybridization, one or more single stranded overhangs, such overhangs shalt not be regarded as mismatches with regard to the determination of complementarily. For example, a dsRNA comprising one oligonucleotide 21 nucleotides in length and another oligonucleotide 23 nucleotides in length, wherein the longer oligonucleotide comprises a sequence of 21 nucleotides that is fully complementary to the shorter oligonucleotide, may yet be referred to as “fully complementary” for the purposes of the invention. “Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled.

The terms “complementary”, “fully complementary” and “substantially complementary” herein may be used with respect to the base matching between the sense strand and the antisense strand of a dsRNA, or between the antisense strand of a dsRNA and a target sequence, as will be understood from the context of their use.

As used herein, a polynucleotide which is “substantially complementary to at least part of” a messenger RNA (mRNA) refers to a polynucleotide that is substantially complementary to a contiguous portion of the mRNA of interest.

Controlled-Release Component: As used herein, the term “controlled-release component” refers to a composition or compound, including, but not limited to, polymers, polymer matrices, gels, permeable membranes, liposomes, microspheres, or the like, or a combination thereof, that facilitates the controlled-release of a composition or composition combination.

Conservative Changes: As used herein, when referring to mutations in a nucleic acid molecule, “conservative changes” are those in which at least one codon in the protein-coding region of the nucleic acid has been changed such that at least one amino acid of the polypeptide encoded by the nucleic acid sequence is substituted with a another amino acid having similar characteristics. Examples of conservative amino acid substitutions are ser for ala, thr, or cys; lys for arg; gin for asn, his, or lys; his for asn; glu for asp or lys; asn for his or gin; asp for gin; pro for gly; leu for ile, phe, met, or vat; val for ile or leu; ile for leu, met, or val; arg for lys; met for phe; tyr for phe or trp; thr for ser; trp for tyr; and phe for tyr.

Double-Stranded RNA or dsRNA: The term “double-stranded RNA” or “dsRNA”, as used herein, refers to a complex of ribonucleic acid molecules, having a duplex structure comprising two anti-parallel and substantially complementary, as defined above, nucleic acid strands. The two strands forming the duplex structure may be different portions of one larger RNA molecule, or they may be separate RNA molecules. Where the two strands are part of one larger molecule, and therefore are connected by an uninterrupted chain of nucleotides between the 3′-end of one strand and the 5′-end of the respective other strand forming the duplex structure, the connecting RNA chain is referred to as a “hairpin loop” and the entire structure is referred to as a “short hairpin RNA” or “shRNA”. Where the two strands are connected covalently by means other than an uninterrupted chain of nucleotides between the 3′-end of one strand and the 5′-end of the respective other strand forming the duplex structure, the connecting structure is referred to as a “linker”. In various aspects, the linker can include the sequences AUG, CCC, ETUCG, CCACC, CTCGAG, AAGCUU, CCACACC, and UUCAAGAGA. The RNA strands may have the same or a different number of nucleotides. The maximum number of base pairs is the number of nucleotides in the shortest strand of the dsRNA minus any overhangs that are present in the duplex. In addition to the duplex structure, a dsRNA may comprise one or more nucleotide overhangs.

Fragment: A “fragment” of a nucleic acid is a portion of a nucleic acid that is less than full-length and comprises at least a minimum length capable of hybridizing specifically with a native nucleic acid under stringent hybridization conditions. The length of such a fragment is preferably at least 15 nucleotides, more preferably at least 20 nucleotides, and most preferably at least 30 nucleotides of a native nucleic acid sequence, A “fragment” of a polypeptide is a portion of a polypeptide that is less than full-length (e.g., a polypeptide consisting of 5, 10, 15, 20, 30, 40, 50, 75, 100 or more amino acids of a native protein), and preferably retains at least one functional activity of a native protein.

Functional Activity: As used herein, the term “functional activity” refers to a protein having any activity associated with the physiological function of the protein.

Gene: As used herein, the term “gene” means a nucleic acid molecule that codes for a particular protein, or in certain cases, a functional or structural RNA molecule.

Homolog: As used herein, the term “homolog” refers to a target gene encoding a target polypeptide isolated from an organism other than a human being.

Introducing Into a Cell: “Introducing into a cell”, when referring to a dsRNA, means facilitating uptake or absorption into the cell, as is understood by those skilled in the art. Absorption or uptake of dsRNA can occur through unaided diffusive or active cellular processes, or by auxiliary agents or devices. The meaning of this term is not limited to cells in vitro; a dsRNA may also be “introduced into a cell”, wherein the cell is part of a living organism. In such instance, introduction into the cell will include the delivery to the organism. For example, for in vivo delivery, dsRNA can be injected into a tissue site or administered systemically. In vitro introduction into a cell includes methods known in the art such as electroporation and lipofection.

Labeled: The term “labeled,” with regard to a probe or antibody, is intended to encompass direct labeling of the probe or antibody by coupling (i.e., physically linking) a detectable substance to the probe or antibody.

Native: When referring to a nucleic acid molecule or polypeptide, the term “native” refers to a naturally-occurring (e.g., a “wild-type”) nucleic acid or polypeptide.

Neuroimmune Activation: As used herein, the term “neuroimmune activation” refers to glial cell activation and release of proinflammatory cytokines such as tumor necrosis factor-α, IL-1β, and IL-6.

Nucleic Acid or Nucleic Acid Molecule: As used herein, the term “nucleic acid” or “nucleic acid molecule” means a chain of two or more nucleotides such as RNA (ribonucleic acid) and DNA (deoxyribonucleic acid). A “purified” nucleic acid molecule is one that is substantially separated from other nucleic acid sequences in a cell or organism in which the nucleic acid naturally occurs (e.g., 30%, 40%, 50%, 60%, 70%, 80%, 90%, 95%, 96%, 97%, 98%, 99% or 100% free of contaminants). The term includes, e.g., a recombinant nucleic acid molecule incorporated into a vector, a plasmid, a virus, or a genome of a prokaryote or eukaryote. Examples of purified nucleic acids include cDNAs, fragments of genomic nucleic acids, nucleic acids produced polymerase chain reaction (PCR), nucleic acids formed by restriction enzyme treatment of genomic nucleic acids, recombinant nucleic acids, and chemically synthesized nucleic acid molecules. A “recombinant” nucleic acid molecule is one made by an artificial combination of two otherwise separated segments of sequence, e.g., by chemical synthesis or by the manipulation of isolated segments of nucleic acids by genetic engineering techniques.

Nucleotide Overhang: As used herein, a “nucleotide overhang” refers to the unpaired nucleotide or nucleotides that protrude from the duplex structure of a dsRNA when a 3′-end of one strand of the dsRNA extends beyond the 5′-end of the other strand, or vice versa. “Blunt” or “blunt end” means that there are no unpaired nucleotides at that end of the dsRNA, i.e., no nucleotide overhang. A “blunt ended” dsRNA is a dsRNA that has no nucleotide overhang at either end of the molecule.

Operably Linked: As used herein, the term “operably linked” refers to a first nucleic-acid sequence physically linked with a second nucleic acid sequence creating a functional relationship with the second nucleic acid sequence. For example, a promoter is operably linked to a coding sequence if the promoter affects the transcription or expression of the coding sequence. Generally, operably linked nucleic acid sequences are contiguous and, where necessary to join two protein coding regions, in reading frame.

Opiate or Opioid: As used herein, the terms “opiate” and “opioid” are used to refer to any of a variety of analgesic agents. The best-known example of an opiate is morphine. Opiates operate by mimicking natural peptides such as enkephalins and endorphins to stimulate one or more of the μ-, δ-, and κ-receptor systems in the nervous system. Opioids are commonly used in the clinical management of severe pain, including chronic severe pain such as that experienced by cancer patients. (Gilman et al., 1980, Goodman and Gilman's. The Pharmacological Basis of Therapeutics, Chapter 24:494-534, Pub. Pergamon Press: hereby incorporated by reference). Opioids include morphine and morphine-like homologs, including, for example, the semisynthetic derivatives codeine (methylmorphine) and hydrocodone (dihydrocodeinone), among many other such derivatives. A non-limiting list of opioid analgesic agents that may be utilized in the present invention includes: alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, diamorphone, dihydrocodeine, dihydromorphine, dimenoxadol, dimepheptanol, dimethylthiambutene, dioxaphetylbutyrate, dipipanone, eptazocine, ethoheptazine, ethylmethylthiambutene, ethylmorphine, etonitazene fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propiram, propoxyphene, sufentanil, tilidine, tramadol, salts thereof, complexes thereof mixtures of any of the foregoing, mixed μ-agonists/antagonists, μ-antagonist combinations salts or complexes thereof, and the like. In certain aspect of the invention, the opioid analgesic is a μ- or κ-opioid agonist. In additional aspects of the invention, the opioid analgesic is a selective κ-agonist.

In certain other aspects of the invention, the opioid analgesic is selected from codeine, hydromorphone, hydrocodone, oxycodone, dihydrocodeine, dihydromorphine, diamorphone, morphine, tramadol, oxymorphone salts thereof or mixtures thereof.

Pharmaceutically Acceptable: As used herein, the term “pharmaceutically acceptable” means approved by a regulatory agency of the Federal or a state government or listed in the U.S. Pharmacopeia or other generally recognized pharmacopeia for use in animals, and more particularly in humans.

Pharmaceutically Acceptable Carrier: As used herein, the term “pharmaceutically acceptable carrier” refers to a diluent, adjuvant, excipient, or vehicle with which a composition is administered. Such carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Water is a preferred carrier when a composition is administered intravenously. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A composition, if desired, can also contain minor amounts of wetting or emulsifying agents, or pH buffering agents such as acetates, citrates or phosphates, Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier.

Pharmaceutically Acceptable Salt: As used herein, the term “pharmaceutically acceptable salt” includes those salts of a pharmaceutically acceptable composition formed with free amino groups such as those derived from hydrochloric, phosphoric, acetic, oxalic, tartaric acids, and those formed with free carboxyl groups such as those derived from sodium, potassium, ammonium, calcium, ferric hydroxides, isopropylamine, triethylamine, 2-ethylamino ethanol, histidine, and procaine. If the composition is basic, salts may be prepared from pharmaceutically acceptable non-toxic acids including inorganic and organic acids. Such acids include acetic, benzene-sulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. Particularly preferred are besylate, hydrobromic, hydrochloric, phosphoric and sulfuric acids. If the composition is acidic, salts may be prepared from pharmaceutically acceptable organic and inorganic bases. Suitable organic bases include, but are not limited to, lysine, chloroprocaine, choline, diethanolamine, ethylenediamine, meglumine (N-methylglucamine) and procaine. Suitable inorganic bases include, but are not limited to, alkaline and earth-alkaline metals such as aluminum, calcium, lithium, magnesium, potassium, sodium and zinc.

Pro-drug: As used herein, the term “pro-drug” refers to any composition which releases an active drug in vivo when such a composition is administered to a mammalian subject. Pro-drugs can be prepared, for example, by functional group modification of a parent drug. The functional group may be cleaved in vivo to release the active parent drug compound. Pro-drugs include, for example, compounds in which a group that may be cleaved in vivo is attached to a hydroxy, amino or carboxyl group in the active drug. Examples of pro-drugs include, but are not limited to esters (e.g., acetate, methyl, ethyl, formate, and benzoate derivatives), carbamates, amides and ethers. Methods for synthesizing such pro-drugs are known to those of skill in the art.

Protein or Polypeptide: As used herein, “protein” or “polypeptide” mean any peptide-linked chain of amino acids, regardless of length or post-translational modification, e.g., glycosylation or phosphorylation, A “purified” polypeptide is one that is substantially separated from other polypeptides in a cell or organism in which the polypeptide naturally occurs (e.g., 30%, 40%, 50%, 60%, 70%, 80%, 90%, 95%, 96%, 97%, 98%, 99% or 100% free of contaminants).

Purified substance: A “purified” substance is one that is substantially separated from other undesired substances such as contaminants that may naturally occur with the substance (e.g., 30%, 40%, 50%, 60%, 70%, 80%, 90%, 95%, 96%, 97%, 98%, 99% or 100% free of contaminants).

Sense Strand: The term “sense strand,” as used herein, refers to the strand of a dsRNA that includes a region that is substantially complementary to a region of the antisense strand.

Sequence Identity: As used herein, “sequence identity” means the percentage of identical subunits at corresponding positions in two sequences when the two sequences are aligned to maximize subunit matching, i.e., taking into account gaps and insertions. Sequence identity is present when a subunit position in both of the two sequences is occupied by the same nucleotide or amino acid, e.g., if a given position is occupied by an adenine in each of two DNA molecules, then the molecules are identical at that position. For example, if 9 positions in a sequence 10 nucleotides in length are identical to the corresponding positions in a second 10-nucleotide sequence, then the two sequences have 90% sequence identity. Percent sequence identity of an antisense compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

Silence and Inhibit the Expression Of: The terms “silence” and “inhibit the expression of,” in as far as they refer to a gene herein refer to the at least partial suppression of the expression of that gene as manifested by a reduction of the amount of mRNA transcribed from that gene, which may be isolated from a first cell or group of cells in which the gene is transcribed and which has or have been treated such that the expression of the corresponding gene product is inhibited, as compared to a second cell or group of cells substantially identical to the first cell or group of cells but which has or have not been so treated (control cells). The degree of inhibition is usually expressed in terms of:

$$\frac{mRNA in control cells - mRNA in treated cells}{mRNA in control cells} \times 100$$

Alternatively, the degree of inhibition may be given in terms of a reduction of a parameter that is functionally linked to gene transcription, e.g. the amount of protein encoded by a gene that is secreted by a cell, or found in solution after lysis of such cells, or the number of cells displaying a certain phenotype. In principle, gene silencing may be determined in any cell expressing the target, either constitutively or by genomic engineering, and by any appropriate assay. However, when a reference is needed in order to determine whether a given dsRNA inhibits the expression of a gene by a certain degree and therefore is encompassed by the instant invention, the assays provided in the Examples below shall serve as such reference. For example, in certain instances, expression of a gene is suppressed by at least about 20%, 21%, 22%, 23%, 24%, 25%, 26%, 27%, 28%, 29%, 30%, 31%, 32%, 33%, 34%, 35%, 36%, 37%, 38%, 19%, 40%, 41%, 42%, 43%, 44%, 45%, 46%, 47%, 48%, 49% or 50% by administration of the double-stranded oligonucleotide of the invention. In various aspects, a gene is suppressed by at least about 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79% or 80% by administration of the double-stranded oligonucleotide of the invention. In various aspects, a gene is suppressed by at least about 81%, 82%, 83%, 84%, 85%, 86%, 87%, 88%, 89%, 90%, 91%, 92%, 93%, 94%, 95%, 96%, 97%, 98%, 99% or 100% by administration of the double-stranded oligonucleotide of the invention.

Silent and Conservative: When referring to mutations in a nucleic acid molecule, “silent” changes are those that substitute of one or more base pairs in the nucleotide sequence, but do not change the amino acid sequence of the polypeptide encoded by the sequence. “Conservative” changes are those in which at least one codon in the protein-coding region of the nucleic acid has been changed such that at least one amino acid of the polypeptide encoded by the nucleic acid sequence is substituted with a another amino acid having similar characteristics. Examples of conservative amino acid substitutions are ser for ala, thr, or cys; lys for arg; gin for asn, his, or lys; his for asn; glu for asp or lys; asn for his or gin; asp for glu; pro for gly; leu for ile, phe, met, or vat; val for ile or leu; ile for leu, met, or val; arg for lys; met for phe; tyr for phe or trp; thr for ser; trp for tyr; and phe for tyr.

Strand Comprising a Sequence: As used herein, the term “strand comprising a sequence” refers to an oligonucleotide comprising a chain of nucleotides that is described by the sequence referred to using the standard nucleotide nomenclature.

Stringent Hybridization Conditions or Stringent Conditions: As used herein, the term “stringent hybridization conditions” or “stringent conditions” refers to conditions under which a compound of the invention will hybridize to its target sequence, but to a minimal number of other sequences. Stringent conditions are sequence-dependent and will be different in different circumstances and in the context of this invention, “stringent conditions” under which oligomeric compounds hybridize to a target sequence are determined by the nature and composition of the oligomeric compounds and the assays in which they are being investigated. For example, hybridization conducted under “low stringency conditions” means in 10% formamide, 5×Denhart's solution, 6×SSPE, 0.2% SDS at 42° C., followed by washing in 1×SSPE, 0.2% SDS, at 50° C.; “moderate stringency conditions” means in 50% formamide, 5×Denhart's solution, 5×SSPE, 0.2% SDS at 42° C., followed by washing in 0.2×SSPE, 0.2% SDS, at 65° C.; and “high stringency conditions” means in 50% formamide, 5×Denhart's solution, 5×SSPE, 0.2% SDS at 42° C., followed by washing in 0.1×SSPE, and 0.1% SDS at 65° C.

Subject: As used herein, the terms “subject” and “subjects” refer to any mammal, including a human mammal. Human subjects include any human who is at risk of developing, or who has developed, opiate induced tolerance or hyperalgesia. This includes any subject who will be administered an opiate, whether the subject has previously received an opiate or not, and whether the subject has previously exhibited signs or symptoms of opiate induced tolerance or hyperalgesia or not. Subjects particularly at risk of developing tolerance are those who require multiple doses of opiates, such as subjects suffering from chronic pain.

Also included in the definitions of “subject” and “subjects” are those who are either already addicted to opiates or who are at risk of addiction to opiates. Subjects addicted to opiates may include humans who have self-administered and/or misused opiates, as well as subjects suffering from hyperalgesia due to opiate withdrawal. Subjects at highest risk for developing opiate induced tolerance or addiction include those subjects who have been administered, or have self-administered, opiates over a prolonged period of time.

Non-human animal subjects may include, but are not limited to, mammals such as primates, mice, pigs, cows, cats, goats, rabbits, rats, guinea pigs, hamsters, horses, sheep, dogs, and the like. Such animals may be companion animals, as in the case of dogs and cats, for example, or may be trained animals including therapy animals such as a therapy dog. Also included are service animals, such as dogs that assist persons who are in need of assistance due to loss or impairment of sight, hearing, or other senses. Further, non-human subjects may include working animals such as dogs or other animals trained for security or rescue work. Also included are animals trained or maintained for procreation or entertainment purposes, including purebred animal breeds, racehorses, or workhorses. Animals that are genetically-engineered are likewise included, regardless of the purposes of the genetic engineering, as are rare or exotic animals, including zoo animals and wild animals.

Target Sequence: As used herein, “target sequence” refers to a contiguous portion of the nucleotide sequence of an mRNA molecule formed during the transcription of a gene, including mRNA that is a product of RNA processing of a primary transcription product. The target sequence of any given RNAi agent of the invention means an mRNA-sequence of X nucleotides that is targeted by the RNAi agent by virtue of the complementarity of the antisense strand of the RNAi agent to such sequence and to which the antisense strand may hybridize when brought into contact with the mRNA, wherein X is the number of nucleotides in the antisense strand plus the number of nucleotides in a single-stranded overhang of the sense strand, if any.

Therapeutically Effective Amount: As used herein, the term “therapeutically effective amount” refers to those amounts that, when administered to a population of subjects will have a desired therapeutic effect, e.g. an amount that will cure, prevent, inhibit, or at least partially arrest or partially prevent a target disease or condition. Alternatively, a “therapeutically effective amount” may be administered to a particular subject in view of the nature and severity of that subject's disease or condition. A therapeutically effective amount with respect to an agent that inhibits ceramide biosynthesis means an amount sufficient to inhibit ceramide biosynthesis upon administration of the agent to a subject. An analgesic amount refers to an amount of a substance that produces a pain-relieving effect when administered to naïve subjects who have not previously received the substance and who have not developed a tolerance to the substance. A sub-analgesic amount refers to an amount of a pain-relieving substance that is less an analgesic amount of the substance.

Therapeutically Effective Time: As used herein, the term “Therapeutically Effective Time” refers to the interval of time between administration of a therapeutic agent of the present invention (e.g. a ceramide biosynthesis inhibitor) and administration of an opiate in co-administration treatment regimens where the therapeutic agent is administered prior to an opiate, concurrently with an opiate, or subsequent to an opiate. A therapeutically effective time may be determined for a general population of subjects. Alternatively, a therapeutically effective time may be determined empirically in each subject by a medical practitioner who may consider among other medically-related indicators, a subjects ceramide levels, or ceramide levels from historical data of similar subjects. Non-limiting examples of a therapeutically effective times include; less than about 15 minutes; about 15 minutes, from about minutes to about one hour; from about 1 to about 2 hours; from about 2 to about 3 hours; frorn about 3 to about 4 hours; from about 4 to about 5 hours; from about 5 to about 6 hours; from about 6 to about 7 hours; from about 7 to about 8 hours; from about 8 to about 9 hours; from about 9 to about 10 hours; from about 10 to about 12 hours; from about 12 to about 14 hours; from about 14 to about 16 hours; from about 16 to about 20 hours; from about 20 to about 24 hours; from about 1 to about 2 days; from about 2 to about 3 days; from about 3 to about 6 days; and more than 6 days.

Transformed, Transfected or Transgenic: A cell, tissue, or organism into which has been introduced a foreign nucleic acid, such as a recombinant vector, is considered “transformed,” “transfected,” or “transgenic.” A “transgenic” or “transformed” cell or organism also includes progeny of the cell or organism, including progeny produced from a breeding program employing such a “transgenic” cell or organism as a parent in a cross.

Vector: As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors.”

“G,” “C,” “A”, “T” and “U” (irrespective of whether written in capital or small letters) each generally stand for a nucleotide that contains guanine, cytosine, adenine, thymine, and uracil as a base, respectively. However, it will be understood that the term “ribonucleotide” or “nucleotide” can also refer to a modified nucleotide, as further detailed below, or a surrogate replacement moiety. The skilled person is well aware that guanine, cytosine, adenine, thymine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of the invention by a nucleotide containing, for example, inosine.

Compositions positions and Methods for Conditions Associated with Ceramide Biosynthesis

The present invention relates to inhibition of ceramide biosynthesis, or otherwise blocking the action of cermide, to treat, prevent, and inhibit biological conditions that are mediated by ceramide, particularly opioid antinociceptive tolerance, nitroxidative stress, and neuroimmune activation. The invention is also directed to methods of detecting ceramide inhibitors. The invention further relates to polynucleotides and polypeptides, including double-stranded RNA (dsRNA) compounds such as siRNAs and shRNAs capable of inhibiting the expression of components of the ceramide biosynthesis pathway.

### Ceramide

Ceramides are a family of lipids composed of sphingosine and a fatty acid. Ceramide synthesis in the body occurs via one of three major pathways: the de novo pathway, the sphingomyelin pathway, and the salvage pathway. The de novo pathway results in ceramide synthesis from less complex molecules in the body. The sphingomyelin pathway produces ceramide through the breakdown of sphingomyelin mediated by the enzyme sphingomyelinase, Ceramide is produced via the salvage pathway by the breakdown of complex sphingolipids into sphingosine, which is then used to form ceramide.

The inventor of the present invention has discovered that opiate treatment causes an increase in ceramide levels in the subject being treated.

### Ceramide Synthesis Inhibitors

For a review of ceramide synthesis inhibitors, see Delgado et al., 2006, which is hereby incorporate herein by reference and discussed below.

### De Novo Pathway

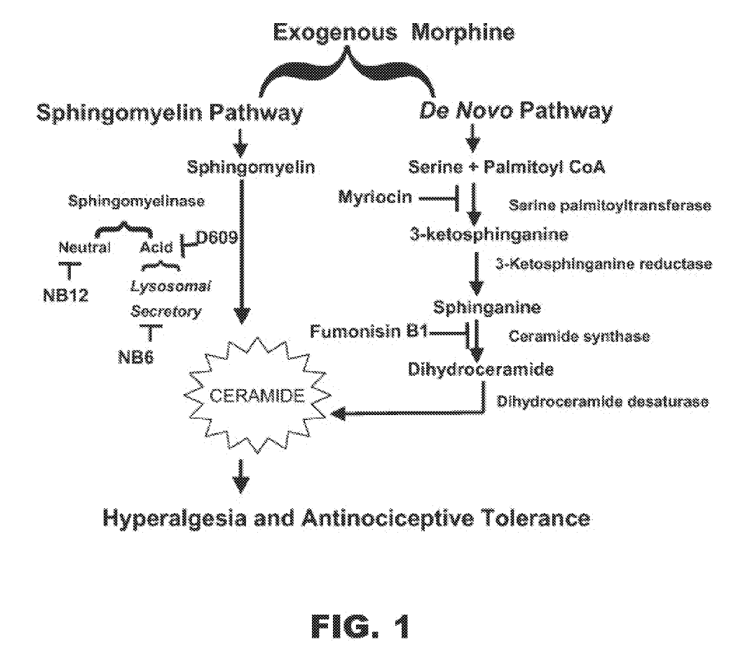

The ceramide de novo pathway includes a series of enzymes that produce ceramide from the starting components serine and palmitoyl CoA. An overview of the pathway is provided in FIG. 1.

Serine palmitoyltransferase (SPT) catalyzes the first step in the synthesis of ceramide in the de novo pathway, which is the production of 3-ketodihydrosphingosine from serine and palmitoyl CoA. By way of example, but not of limitation, inhibitors of SPT include the sphingo-fungins, lipoxamycin, myriocin, L-cycloserine and beta-chloro-L-alanine, as well as the class of Viridiofungins.

Ceramide synthase (CerS) catalyzes the acylation of the amino group of sphingosine, sphinganine, and other sphingoid bases using acyl CoA esters. By way of example, but not of limitation, inhibitors of this enzyme include the Fumonisins, the related AAL-toxin, and australifungins. The Fumonisins family of inhibitors are produced by Fusarium verticillioides and includes Fumonisin B1 (FB1). The N-acylated forms of FB1 are known to be potent CerS inhibitors while the O-deacylated form is less potent. Of the N-acylated forms of FB1, the erythro-, threo-2-amino-3-hydroxy-, and stereoisomers of 2-amino-3,5-dihydroxyoctadecanes are also known as CerS inhibitors. Australifungins from the organism Sporondella australlis is also a potent inhibitor of CerS.

Dihydroceramide desaturase (DES) is the last enzyme in the de novo biosynthesis pathway of ceramide synthesis. At least two different forms, DES1 and DES2, are known. By way of example, but not of limitation, inhibitors of these enzymes include the cyclopropene-containing sphingolipid GT11, as well as a-ketoamide (GT85, GT98, GT99), urea (GT55), and thiourea (GT77) analogs of this molecule.

### Sphingomyelin Pathway

Sphingomyelin hydrolysis by sphingomyelinases (SMases) produces phosphorylcholine and ceramide. At least five isotypes of SMase are known, including acid and neutral forms. Several physiological inhibitors of acid SMase have been described including L-alpha-phosphatidyl-D-myo-inositol-3,5-bisphosphate, a specific acid SMase inhibitor, and L-alpha-phosphatidyl-D-myo-inositol-3,4,5-triphosphate, a non-competitive inhibitor of acid SMase. Ceramide-1-phosphate and sphingosine-1-phosphate have also been described as physiological inhibitors. Glutathione is an inhibitor of neutral SMase at physiological concentrations with a greater than 95% inhibition observed at 5 mM GSH. Compounds that are structurally unrelated to sphingomyelin but function as SMase inhibitors included desipramine, imipramine, SR33557, (3-carbazol-9-yl-propyl)-[2-(3,4-dimethoxy-phenyl)-ethyl)-methyl-amine (NB6), hexanoic acid (2-cyclo-pent-1-enyl-2-hydroxy-1-hydroxy-methyl-ethyl)-amide (NB12) C11AG, and GW4869. Compound SR33557 is a specific acid SMase inhibitor (72% inhibition at 30 μM). The compound NB6 has been reported as an inhibitor of the SMase gene transcription. Inhibitors derived from natural sources include Scyphostatin, Macquarimicin A, and Alutenusin, which are non-competitive inhibitors of neutral SMase, and Chlorogentisylquinone, and Manumycin A, which are irreversible specific inhibitors of neutral SMase. Also described is α-Mangostin, an inhibitor of acid SMase. Scyphostatin analogs with inhibitory proprieties include spiroepoxide 1, Scyphostatin, and Manumycin A sphingolactones. Sphingomyelin analogs with inhibitory proprieties include 3-O-methylsphingomyelin, and 3-O-ethylsphingomyelin.

Table 1, below, provides a non-exhaustive list of exemplary sphingomyelinase inhibitors known in the art.

TABLE 1
Exemplary Sphingomyelinase Inhibitors

| No. | COMPOUND NAME |
|---|---|
| 1 | [3 (10,11-Dihydro-dibenzo[b,f]azepin-5-yl)-N-propyl]-[2 (3,4-dimethoxypenyl)- |
| | ethyl]methylamine |
| 2 | [3 (10,11-Dihydro-dibenzo[b,f]azepin-5-yl)-N-propyl]-[2 (4-methoxyphenyl)- |
| | ethyl]methylamine |
| 3 | [2 (3,4-Dimethoxypheyl)-ethyl]-[3 (2-chlorphenothiazin-10-yl)-N-propyl]-methylamine |
| 4 | [2 (4-Methoxyphenyl)-ethyl]-[3 (2-chlorphenothiazin-10-yl)-N-propyl]-methylamine |
| 5 | [3 (Carbazol-9-yl)-N-propyl]-[2 (3,4-dimethoxyphenyl)-ethyl]methylamine |
| 6 | [3 (Carbazol-9-yl)-N-propyl]-[2 (4-methoxyphenyl)-ethyl]methylamine |
| 7 | [2 (3,4-Dimethoxyphenyl)-ethyl]-[2 (phenothiazin-10-yl)-N-ethyl]-methylamine |
| 8 | [2 (4-Methoxyphenyl)-ethy]-[2 (phenothiazin-10-yl)-N-ethyl]-methylamine |
| 9 | [(3,4-Dimethoxyphenyl)-acetyl]-[3 (2-chlorphenothiazin-10-yl)-N-propyl]-methylamine |
| 10 | n (1-naphthyl)-N′ [2 (3,4-dimethoxyphenyl)-ethyl]-ethyl diamine |
| 11 | n (1-naphthyl)-N[2 (4-methoxyphenyl)-ethyl]-ethyl diamine |
| 12 | n [2 (3,4-Dimethoxyphenyl)-ethyl]-n [1-naphthylmethyl]amine |
| 13 | n [2 (4-Methoxyphenyl)-ethyl]-n [1-naphthylmethyl]amine |
| 14 | [3 (10.11-Dihydro dibenzo[b,f]azepin-5-yl)-N-propyl]-[(4-methoxyphenyl)-acetyl]- |
| | methylamine |
| 15 | [2 (10,11-Dihydro-dibenzo[b,f]azepin-5-yl)-N-ethyl]-[2 (3,4-dimethoxyphenyl)- |
| | ethyl]methylamine |
| 16 | [2 (10,11-Dihydro-dibenzo[b,f]azepin-5-yl)-N-ethyl]-[2 (4-methoxyphenyl)-ethyl]- |
| | methylamine |
| 17 | [2 (10,11-Dihydro-dibenzo[b,f]azepin-5-yl)-N-ethyl]-[(4-methoxyphenyl)-acety-1]- |
| | methylamine |
| 18 | n [2 (Carbazol-9-yl)-N-ethyl]-N′ [2 (4-methoxyphenyl)-ethyl]piperazine |
| 19 | 1[2 (Carbazol-9-yl)-N-ethyl]-4[2 (4-methoxyphenyl)-ethyl]-3,5-dimethylpiperazine |
| 20 | [2 (4-Methoxyphenyl)-ethyl]-[3 (phenoxazin-10-yl)-N-propyl]-methylamine |
| 21 | [3 (5,6,11,12-Tetrahydrodibenzo[b,f]azocin)-N-propyl]-[3 (4-methoxyphenyl)- |
| | propyl]methylamine |
| 22 | n (5H-Dibenzo [A,D]cycloheptan-5-yl)-N′ [2 (4-methoxyphenyl)-ethyl]-propylene diamine |
| 23 | [2 (Carbazol-9-yl)-N-ethyl]-[2(4-methoxyphenyl)-ethyl]methylamine |

Other compounds or agents shown in the art to reduce ceramide levels include L-carnitine (200 mcg/ml), siylmarin, 1-phenyl-2-decanoylaminon-3-morpholine-1-propanol, 1-phenyl-2-hexdecanoylaminon-3-pyrrolidino-1-propanol, Scyphostatin, L-carnitine, glutathione, human milk bile salt-stimulated lipase, myriocin, cycloserine, Fumonisin 9, PPMP, D609, methylthiodihydroceramide, propanolol, and resveratrol. Agents comprised of polypeptide sequences have also been shown to reduce ceramide levels, as describe in U.S. Pat. No. 7,037,700, incorporated herein by reference.

The foregoing listing of agents that reduce ceramide levels is non-exhaustive. It will be apparent to one of skill in the art that analogs or fragments of the inhibitors described herein may also possess inhibitory properties. In addition to the agents described herein, the present invention may also be practiced using agents that decrease ceramide pathway metabolic enzymes or increase ceramide catabolic enzymes. These include, but are not limited to, agents that modify or regulate transcriptional or translational activity, or that otherwise degrade, inactivate, or protect these enzymes.

### Screening Methods

The present invention additionally provides a method of screening for an agent that reduces, prevents or delays the development of tolerance to, and/or physical dependence on, an opioid drug that targets an opioid receptor. The method entails contacting a cell comprising the opioid receptor, with a test agent and then determining whether the test agent inhibits biosynthesis of ceramide, for example, by measuring enzyme levels in a pathway for biosynthesis of ceramide. In various embodiments, the method may involve contacting the cell with an opioid drug. In various embodiments in which the cell is contacted with a test compound and an opioid drug, the cell may be contacted with the test compound prior to contacting the cell with the opioid drug, for example from about 5 minutes to about 30 minutes, from about 30 minutes to about 1 hour, from about 1 hour to about 2 hours, from about 2 hours to about 6 hours or from about 6 hours to about 24 hours prior to contact the cell with the opioid drug or any time therebetween; substantially at the same time as contacting the cell with the opioid drug or after contacting the cell with the opioid drug, for example from about 5 minutes to about 30 minutes, from about 30 minutes to about 1 hour, from about 1 hour to about 2 hours, from about 2 hours to about 6 hours or from about 6 hours to about 24 hours after contact the cell with the opioid drug or any time therebetween. In various embodiments, the method may involve determining whether the test agent reduces or prevents an increase in ceramide levels elicited by the opioid drug in addition to or as an alternative to determining whether the test agent inhibits biosynthesis of ceramide. The method may further involve selecting the test agent as an agent that may reduce, prevent or delay the development of tolerance to, and/or physical dependence on, the opioid drug if the test agent inhibits synthesis of ceramide and/or reduces or prevents an increase in ceramide levels elicited by the opioid drug.

Cells useful in the screening methods of the invention comprise an opioid receptor such as a μ-opioid receptor, δ-opioid receptor or a κ-opioid receptor. Determining whether the test agent inhibits biosynthesis of ceramide may reduce, prevent or delay the development of tolerance to, and/or physical dependence on, the opioid drug be achieved by contacting the cell with an opioid drug and then measuring activity of one or more enzymes in the biosynthetic pathway for ceramide in the absence and presence of the test compound. Examples of such enzymes include a sphingomyelinase, a serine palmitoyltransferase, a 3-ketosphinganine reductase, ceramide synthase or a dihydroceramide desaturase. A test agent that reduces or prevents an increase in enzyme activity elicited by the opioid drug may be selected as a compound that may reduce, prevent or delay the development of tolerance to, and/or physical dependence on, the opioid drug. The activities of enzymes in the biosynthetic pathway for ceramide may be measured by any of a variety of methods including those described in Example 3 below.

Alternatively, determining may involve contacting the cell with an opioid drug and then measuring the ceramide levels in the absence and presence of the test compound. A test agent that reduces or prevents the increase in ceramide levels elicited hy the opioid drug may be selected. In such studies ceramide levels may be measured by any of a variety of methods, including those described herein. For in vitro studies, ceramide levels may be measured using methods such as thin-layer or high-performance liquid chromatography or mass spectrometry or E. coli diacylglycerol kinase assay (see, for example, Cremesti and Fischl, 2000). For in vivo studies, samples may be obtained from test animals and assay methods described above may be used or, alternatively, immunohistochemistry methods as described in Examples 1 and 3 below may be used.

In some embodiments, the contacting step may be carried out in vitro to facilitate the screening of large numbers of test agents. Briefly, an in vitro screening method may be performed by incubating cells comprising a suitable opioid receptor with an opioid drug and a test agent under conditions designed to provide a ceramide biosynthesis inhibitory concentration of the test agent over the incubation period. After test agent treatment and incubation, the cells may be recovered and assayed as described above.

High-throughput methods may be employed for the screening method of the invention. Such high-throughput methods may utilize any of a variety of testing and assay methods such as those described above. Exemplary assays amenable to high-throughput screening are known in the art. In particular, assay methods involving measurement of ceramide levels have been reported (see, for example, Bektas et al., 2003; Liebisch et al., 1999). Other assay methods known in the art may also be used such as those described below in Examples 1 and 3.

### Test Agent Database

In various embodiments, generally involving the screening of a large number of test agents, the screening method may include the recordation of any test agent of interest that inhibits ceramide biosynthesis and/or reduces or prevents an increase in ceramide levels elicited by the opioid drug, in a database of agents that may reduce, prevent or delay the development of tolerance to, and/or physical dependence on, an opioid drug.

The term “database” refers to a means for recording and retrieving information. In various embodiments, the database also provides means for sorting and/or searching the stored information. The database can employ any convenient medium including, but not limited to, paper systems, card systems, mechanical systems, electronic systems, optical systems, magnetic systems or combinations thereof. In various embodiments, databases include electronic (e.g. computer-based) databases. Computer systems for use in storage and manipulation of databases are well known to those of skill in the art and include, but are not limited to “personal computer systems,” mainframe systems, distributed nodes on an internet or intranet data or databases stored in specialized hardware (e.g. in microchips) and the like.

### Screening Libraries

Many assays for screening candidate or test compounds that decrease or inhibit biosynthesis of ceramide and/or reduce or prevent an increase in ceramide levels elicited by an opioid drug, are available to those of skill in the art. Test compounds can be obtained using any of the numerous approaches in combinatorial library methods, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptides, while the other four approaches encompass peptide, non-peptide oligomer or small molecule libraries of compounds.

Preparation and screening of combinatorial chemical libraries is well known to those of skill in the art. Such combinatorial chemical libraries include, but are not limited to, peptide libraries (see, e.g., U.S. Pat. No. 5,010,175, Furka et al., 1991 and Houghton et al. 1991), Other chemistries for generating chemical diversity libraries are also optionally used. Such chemistries include, but are not limited to: peptoids (PCT Publication No. WO 91/19735), encoded peptides (PCT Publication WO 93/20242), random bio-oligomers (PCT Publication No. WO 92/00091), benzodiazepines (U.S. Pat. No. 5,288,514), diversomers such as hydantoins, benzodiazepines and dipeptides (Hobbs-Dewitt et al., 1993), vinylogous polypeptides (Hagihara et al., 1992), nonpeptidal peptidomimetics with .alpha.-D-glucose scaffolding (Hirschmann et al., 1992), analogous organic syntheses of small compound libraries (Chen et al., 1994), oligocarbamates (Cho et al., 1993), and/or peptidyl phosphonates (Campbell et al., 1994), nucleic acid libraries (see, Berger and Kimmel, Guide to Molecular Cloning Techniques, Methods in Enzymology, volume 152, Academic Press, Inc., San Diego, Calif., Sambrook, supra, and Ausubel, supra; peptide nucleic acid libraries (see, e.g., U.S. Pat. No. 5,539,083), antibody libraries (see, e.g., Vaughan et al., 1996 and PCT/US96/10287), carbohydrate libraries (see, e.g., Liang et al., 1996 and U.S. Pat. No. 5,593,853), small organic molecule libraries (see, e.g., benzodiazepines, Baum, 1993; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

### Neuroimmune Activation

Neuroimmune activation is the activation of cells that interact with the nervous system. The process includes activation of spinal glial cells, and can result in an increased production of cytokines, cellular adhesion molecules, chemokines, and surface antigens that can enhance an immune cascade. Among the cytokines upregulated by the neuroimmune activation process are TNF-α, IL-1β, and IL-6. Unless otherwise indicated, the term “neuroimmune activation” as used herein will retain the definition set forth in the Definitions section of this writing.

Neuroimmune activation contributes to morphine antinociceptive tolerance. Thus, anti-cytokine approaches to dealing with antinociceptive tolerance, as well as inhibitors of glial cell metabolism, block morphine-induced hyperalgesia and antinociceptive tolerance. The inventor of the present invention has discovered that ceramide plays a novel role as a signaling mediator in neuroimmune activation, and describes the importance of NF-κB in this process.

### Nitroxidative Stress/Peroxynitrite

Neuronal and epithelial cells in the brain produce the signaling molecule nitric oxide (NO) from L-arginine and oxygen. The process is mediated by the enzyme nitric oxide synthase (NOS). NO reacts rapidly with superoxide (O₂⁻) to produce peroxynitrite (ONOO⁻), a powerful oxidant, pro-inflammatory, and primary component of nitroxidative stress. Nitroxidative stress can initiate a cascade of redox reactions that can trigger apoptosis and a number of cytotoxic effects. Peroxynitrite contributes to the development of morphine antinociceptive tolerance through spinal apoptosis and increased production of TNF-α, IL-1β, and IL-6.

The inventor of the present invention has discovered that ceramide plays a novel role in the production of peroxynitrite.

### Methods of Treatment

The present invention provides for both prophylactic and therapeutic methods of treating a subject at risk of susceptible to, or having a disorder or condition associated with ceramide biosynthesis. Examples of such disorders or conditions include opioid tolerance, nitroxidative stress (and resulting disorders and conditions), and neuroimmune activation (and resulting disorders and conditions).

### Treatment of Diseases, Disorders, and Conditions

Diseases, disorders, and conditions characterized by increased ceramide biosynthesis may be treated with therapeutics that antagonize (i.e. reduce or inhibit) the production of ceramide. Antagonists may be administered in a therapeutic or prophylactic manner. Such antagonists are included broadly herein as agents that reduce or inhibit ceramide biosynthesis, and may include, but are not limited to: 1) proteins or polypeptides that reduce or inhibit ceramide biosynthesis, including analogs, derivatives, fragments, or homologs thereof; 2) antibodies to proteins or peptides involved in the biosynthesis of ceramide; 3) nucleic acids; or 4) administration of antisense nucleic acid or dsRNAs.

Diseases, disorders, or conditions that are characterized by increased levels of ceramide may also be treated with agents that inhibit the downstream action of ceramide that has already been produced.

A non-limiting method of determining ceramide levels in a subject includes the following: lipid extracts from blood, plasma, or spinal fluid may be prepared by back-washing with the artificial upper phase and drying under nitrogen prior to storage in chloroform under nitrogen until Electrospray Tonisation Mass Spectrometry (ESI-MS) analysis, Lipid extracts may be mixed with methanol containing 10 mM NaOH prior to direct infusion into the ESI-MS source at a flow rate of 3 μl per minute, Ceramides can be directly analyzed in the negative-ion ESI-MS. Tandem mass spectrometry: of ceramides after ESI can be performed with collision energy of 2.5 mTorr (argon). With tandem mass spectrometry, ceramides can be detected by the neutral loss of m/z 256.2. Typically, a five minute period of signal averaging for each ceramide sample, or a ten minute period of signal averaging for each tandem mass spectrum of a lipid extract in the profile mode, should be employed. Ceramide molecular species can be directly quantitated by comparisons of ion peak intensities with that of internal standard (i.e. 17:0 ceramide) in both ESI-MS and ESI-MS-MS analyses after a correction for ¹³C isotope effects.

Ceramide levels may be determined through any number of techniques known to those skilled in the art, including but not limited to thin layer chromatography, high-pressure liquid chromatography, mass spectrometry, immunochemical-based assays and enzyme-based assays, including those using ceramide kinase or diacylglycerol kinase as described by Bektas et al. (2003) and Modrak (2005).

### Antibodies as Therapeutic or Prophylactic Agents

Antibodies to proteins or peptides involved in the biosynthesis of ceramide may be used in accordance with the teachings of the present invention. Exemplary antibodies include those antibodies that inhibit activity of enzymes of the sphingomyelin pathway, antibodies that inhibit activity of enzymes of the de novo pathway, or any combination thereof.

Thus the present invention includes the use of Antibodies (Abs) and antibody fragments, such as Fab or (Fab)₂ that bind immunospecifically to any epitope of an enzyme in the pathway for biosynthesis of ceramide. Examples of such ceramide biosynthesis enzymes include a sphingomyelinase, a serine palmitoyltransferase, a 3-ketosphinganine reductase, ceramide synthase or a dihydroceramide desaturase. An “Antibody” (Ab) may include single Abs directed against a ceramide biosynthesis enzyme, Ab compositions with poly-epitope specificity, single chain Abs, and fragments of Abs. A “monoclonal antibody” is obtained from a population of substantially homogeneous Abs, i.e., the individual Abs comprising the population are identical except for possible naturally-occurring mutations that may be present in minor amounts. Exemplary Abs include polyclonal (pAb), monoclonal (mAb), humanized, bi-specific (bsAb), and heteroconjugate Abs. Antibodies can be produced by any known method in the art or obtained commercially.

### Monovalent Abs

The Abs may be monovalent Abs that consequently do not cross-link with each other. For example, one method involves recombinant expression of Ig light chain and modified heavy chain. Heavy chain truncations generally at any point in the Fc region will prevent heavy chain cross-linking. Alternatively, the relevant cysteine residues are substituted with another amino acid residue or are deleted, preventing crosslinking. In vitro methods are also suitable for preparing monovalent Abs. Abs can be digested to produce fragments, such as Fab fragments.

### Humanized and Human Abs

Antibodies to a ceramide biosynthesis enzyme may further comprise humanized or human Abs. Humanized forms of non-human Abs are chimeric Igs, Ig chains or fragments (such as Fv, Fab, Fab′, F(ab)′2 or other antigen-binding subsequences of Abs) that contain minimal sequence derived from non-human Ig.

Generally, a humanized antibody has one or more amino acid residues introduced from a non-human source. These non-human amino acid residues are often referred to as “import” residues, which are typically taken from an “import” variable domain. Humanization is accomplished by substituting rodent CDRs or CDR sequences for the corresponding sequences of a human antibody. Such “humanized” Abs are chimeric Abs, wherein substantially less than an intact human variable domain has been substituted by the corresponding sequence from a non-human species. In practice, humanized Abs are typically human Abs in which some CDR residues and possibly some FR residues are substituted by residues from analogous sites in rodent Abs. Humanized Abs include human Igs (recipient antibody) in which residues from a complementary determining region (CDR) of the recipient are replaced by residues from a CDR of a non-human species (donor antibody) such as mouse, rat or rabbit, having the desired specificity, affinity and capacity. In some instances, corresponding non-human residues replace Fv framework residues attic human Ig. Humanized Abs may comprise residues that are found neither in the recipient antibody nor in the imported CDR or framework sequences. In general, the humanized antibody comprises substantially all of at least one, and typically two, variable domains, in which most if not all of the CDR regions correspond to those of a non-human Ig and most if not all of the FR regions are those of a human Ig consensus sequence. The humanized antibody optimally also comprises at least a portion of an Ig constant region (Fe), typically that of a human Ig.

Human Abs can also be produced using various techniques, including phage display libraries and the preparation of human mAbs. Similarly, introducing human Ig genes into transgenic animals in which the endogenous Ig genes have been partially or completely inactivated can be exploited to synthesize human Abs. Upon challenge, human antibody production is observed, which closely resembles that seen in humans in all respects, including gene rearrangement, assembly, and antibody repertoire.

Bi-Specific mAbs

Bi-specific Abs are monoclonal, preferably human or humanized, that have binding specificities for at least two different antigens. For example, one binding specificity may be to a ceramide biosynthesis enzyme and the other is for any antigen of choice, preferably a cell-surface protein or receptor or receptor subunit. Traditionally, the recombinant production of bi-specific Abs is based on the co-expression of two Ig heavy-chain/light-chain pairs, where the two heavy chains have different specificities. Because of the random assortment of Ig heavy and light chains, the resulting hybridomas (quadromas) produce a potential mixture of ten different antibody molecules, of which only one has the desired bi-specific structure. The desired antibody can be purified using affinity chromatography or other techniques.

To manufacture a bi-specific antibody, variable domains with the desired antibody-antigen combining sites are fused to Ig constant domain sequences. The fusion is preferably with an Ig heavy-chain constant domain, comprising at least part of the hinge, CH2, and CH3 regions. Preferably, the first heavy-chain constant region (CH1) containing the site necessary for light-chain binding is in at least one of the fusions. DNAs encoding the Ig heavy-chain fusions and, if desired, the Ig light chain, are inserted into separate expression vectors and are co-transfected into a suitable host organism.

The interface between a pair of antibody molecules can be engineered to maximize the percentage of heterodimers that are recovered from recombinant cell culture. The preferred interface comprises at least part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This mechanism increases the yield of the heterodimer over unwanted end products such as homodimers.

Bi-specific Abs can be prepared as full length Abs or antibody fragments (e.g., F(ab′)2 bi-specific Abs). One technique to generate bi-specific Abs exploits chemical linkage. Intact Abs can be proteolytically cleaved to generate F(ab′)2 fragments. Fragments are reduced with a dithiol complexing agent, such as sodium arsenite, to stabilize vicinal dithiols and prevent intermolecular disulfide formation. The generated F(ab′)2 fragments are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bi-specific antibody. The produced bi-specific Abs can be used as agents for the selective immobilization of enzymes.

F(ab′)2 fragments may be directly recovered from E. coli and chemically coupled to form bi-specific Abs. For example, fully humanized bi-specific F(ab′)2 Abs can be produced by methods known to those of skill in the art. Each Fab′ fragment is separately secreted from E. coli and directly coupled chemically in vitro, forming the bi-specific antibody.

Various techniques for making and isolating bi-specific antibody fragments directly from recombinant cell culture have also been described. For example, leucine zipper motifs can be exploited. Peptides from the Fos and Jun proteins are linked to the Fab′ portions of two different Abs by gene fusion. The antibody homodimers are reduced at the hinge region to form monomers and then re-oxidized to form antibody heterodimers. This method can also produce antibody homodimers. The “diabody” technology provides an alternative method to generate bi-specific antibody fragments. The fragments comprise a heavy-chain variable domain (VH) connected to a tight-chain variable domain (VL) by a linker that is too short to allow pairing between the two domains on the same chain. The VH and VL domains of one fragment are forced to pair with the complementary VL and VH domains of another fragment, forming two antigen-binding sites. Another strategy for making bi-specific antibody fragments is the use of single-chain Fv (sFv) dimers. Abs with more than two valences are also contemplated, such as tri-specific Abs.

Exemplary bi-specific Abs may bind to two different epitopes on a given ceramide biosynthesis enzyme or two epitopes on two different ceramide biosynthesis enzymes. Alternatively, cellular defense mechanisms can be restricted to a particular cell expressing the particular ceramide biosynthesis enzyme: an antibody to a ceramide biosynthesis enzyme arm may be combined with an arm that binds to a leukocyte triggering molecule, such as a T-cell receptor molecule CD2, CD3, CD28, or B7), or to Fe receptors for IgG (FcγR), such as FcγRI (CD64), FcγRII (CD32) and FcγRIII (CD16).

### Heteroconjugate Abs

Heteroconjugate Abs, consisting of two covalently joined Abs, have been proposed to target immune system cells to unwanted cells. Abs prepared in vitro using synthetic protein chemistry methods, including those involving cross-linking agents, are contemplated. For example, immunotoxins may be constructed using a disulfide exchange reaction or by forming a thioether bond. Examples of suitable reagents include iminothiolate and methyl-4-mercaptobutyrimidate.

### Nucleic Acids as Therapeutic or Prophylactic Agents

Nucleic acid molecules that inhibit expression of one or more components of a ceramide biosynthesis pathway may be used in accordance with the teachings of the present invention. Exemplary nucleic acids include those nucleic acids that inhibit expression of enzymes of the sphingomyelin pathway, nucleic acids that inhibit expression of enzymes of the de novo pathway, or any combination thereof.

Nucleic acid molecules utilized in the present invention may be in the form of RNA or in the form of DNA (e.g., cDNA, genomic DNA, and synthetic DNA). The DNA may be double-stranded or single-stranded, and if single-stranded may be the coding (sense) strand or non-coding (anti-sense) strand. The coding sequence that encodes any protein or peptide described herein may be identical to a sequence provided in this writing, or it may also be a different coding sequence which, as a result of the redundancy or degeneracy of the genetic code, encodes the same polypeptide as the polynucleotides described herein. Examples of nucleotide codons which provide the same expressed amino acid are summarized in Table 2:

TABLE 2
Nucleotide codons.

| Codon | Full Name | Abbreviation (3 Letter) | Abbreviation (1 Letter) |
|---|---|---|---|
| TTT | Phenylalanine | Phe | F |
| TTC | Phenylalanine | Phe | F |
| TTA | Leucine | Leu | L |
| TTG | Leucine | Leu | L |
| TCT | Serine | Ser | S |
| TCC | Serine | Ser | S |
| TCA | Serine | Ser | S |
| TCG | Serine | Ser | S |
| TAT | Tyrosine | Tyr | Y |
| TAC | Tyrosine | Tyr | Y |
| TAA | Termination | Ter | X |
| TAG | Termination | Ter | X |
| TGT | Cysteine | Cys | C |
| TGC | Cysteine | Cys | C |
| TGA | Termination | Ter | X |
| TGG | Tryptophan | Trp | W |
| CTT | Leucine | Leu | L |
| CTC | Leucine | Leu | L |
| CTA | Leucine | Leu | L |
| CTG | Leucine | Leu | L |
| CCT | Proline | Pro | P |
| CCC | Proline | Pro | P |
| CCA | Proline | Pro | P |
| CCG | Proline | Pro | P |
| CAT | Histidine | His | H |
| CAC | Histidine | His | H |
| CAA | Glutamine | Gln | Q |
| CAG | Glutamine | Gln | Q |
| CGT | Arginine | Arg | R |
| CGC | Arginine | Arg | R |
| CGA | Arginine | Arg | R |
| CGG | Arginine | Arg | R |
| ATT | Isoleucine | Ile | I |
| ATC | Isoleucine | Ile | I |
| ATA | Isoleucine | Ile | I |
| ATG | Methionine | Met | M |
| ACT | Threonine | Thr | T |
| ACC | Threonine | Thr | T |
| ACA | Threonine | Thr | T |
| ACG | Threonine | Thr | T |
| AAT | Asparagine | Asn | N |
| AAC | Asparagine | Asn | N |
| AAA | Lysine | Lys | K |
| AAG | Lysine | Lys | K |
| AGT | Serine | Ser | S |
| AGC | Serine | Ser | S |
| AGA | Arginine | Arg | R |
| AGG | Arginine | Arg | R |
| GTT | Valine | Val | V |
| GTC | Valine | Val | V |
| GTA | Valine | Val | V |
| GTG | Valine | Val | V |
| GCT | Alanine | Ala | A |
| GCC | Alanine | Ala | A |
| GCA | Alanine | Ala | A |
| GCG | Alanine | Ala | A |
| GAT | Aspartate | Asp | D |
| GAC | Aspartate | Asp | D |
| GAA | Glutamate | Glu | E |
| GAG | Glutamate | Glu | E |
| GGT | Glycine | Gly | G |
| GGC | Glycine | Gly | G |
| GGA | Glycine | Gly | G |
| GGG | Glycine | Gly | G |

Examples of such nucleotide substitutions, as shown in Table 2, are those that cause changes in (a) the structure of the polypeptide backbone; (b) the charge or hydrophobicity of the polypeptide; or (c) the bulk of an amino acid side chain. Nucleotide substitutions generally expected to produce the greatest changes in protein properties are those that cause non-conservative changes in codons. Examples of codon changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histadine, for (or by) an electronegative residue, e.g., glutamine or aspartine, or (d) a residue having a bulky side chain, e.g., phenylalanine, for (or by) one not having a side chain, e.g., glycine. Table 3 provides similar possible substitution possibilities:

TABLE 3
Amino Acid Properties

| | 3-letter | 1-letter | |
|---|---|---|---|
| Amino Acid | code | code | Properties |
| Alanine | Ala | A | Aliphatic, hydrophobic, neutral |
| Arginine | Arg | R | polar, hydrophilic, charged (+) |
| Asparagine | Asn | N | polar, hydrophilic, neutral |
| Aspartate | Asp | D | polar, hydrophilic, charged (−) |
| Cysteine | Cys | C | polar, hydrophobic, neutral |
| Glutamine | Gln | Q | polar, hydrophilic, neutral |
| Glutamate | Glu | E | polar, hydrophilic, charged (−) |
| Glycine | Gly | G | aliphatic, neutral |
| Histidine | His | H | aromatic, polar, hydrophilic, |
| | | | charged (+) |
| Isoleucine | Ile | I | Aliphatic, hydrophobic, neutral |
| Leucine | Leu | L | Aliphatic, hydrophobic, neutral |
| Lysine | Lys | K | polar, hydrophilic, charged (+) |
| Methionine | Met | M | hydrophobic, neutral |
| Phenylalanine | Phe | F | aromatic, hydrophobic, neutral |
| Proline | Pro | P | hydrophobic, neutral |
| Serine | Ser | S | polar, hydrophilic, neutral |
| Threonine | Thr | T | polar, hydrophilic, neutral |
| Tryptophan | Trp | W | aromatic, hydrophobic, neutral |
| Tyrosine | Tyr | Y | aromatic, polar, hydrophobic |
| Valine | Val | V | Aliphatic, hydrophobic, neutral |

Naturally occurring allelic variants of a native gene or native mRNAs within the invention are nucleic acids isolated from human tissue that have at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the native gene or native mRNAs, and encode polypeptides having structural similarity to a native protein. Homologs of the native gene or native mRNAs within the invention are nucleic acids isolated from other species that have at least 75% (e.g., 75%, 76%, 77%, 78%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the native gene or native mRNAs, and encode polypeptides having structural similarity to native protein. Public and/or proprietary nucleic acid databases can be searched to identify other nucleic acid molecules having a high percent (e.g., 75%, 85%, 95% or more) sequence identity to the native gene or native mRNAs.

Non-naturally occurring gene or mRNA variants are nucleic acids that do not occur in nature (e.g., are made by the hand of man), comprise a sequence having at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the native gene or native mRNAs, and encode polypeptides having structural similarity to native protein, and preferably retain at least one functional activity. Examples of non-naturally occurring gene variants are those that encode a fragment of native protein, those that hybridize to the native gene or a complement of the native gene under stringent conditions, those that share at least 75% sequence identity with the native gene or a complement thereof, and those that encode a native fusion protein.

Nucleic acids encoding fragments of a native protein within the invention are those that encode, e.g., 2, 3, 4, 5, 10, 25, 50, 100, 150, 200, 250, 300, 350, 400, 450, 500, 550, 600, 650, 700, 750, 800, 900 or more amino acid residues of the native protein. Shorter oligonucleotides (e.g., those of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 53, 54, 55, 56, 57, 58, 59, 60, 61, 62, 63, 64, 65, 66, 67, 68, 69, 70, 71, 72, 73, 74, 75, 76, 77, 78, 79, 80, 100, 125, 150, 200, or 250 base pairs in length) that encode or hybridize with nucleic acids that encode fragments of a native 300, 350, 400, 450, 500, 550, 600, 650, 700, 750, 800, 900 protein can be used as probes, primers, or antisense molecules. Longer polynucleotides (e.g., those of 300, 400, 500, 600, 700, 800, 900, 1000, 1100, 1200, 1300, 1400, 1500, 1600, 1700, 1800, 1900 or 2000 base pairs) that encode or hybridize with nucleic acids that encode fragments of a native protein can also be used in various aspects of the invention. Nucleic acids encoding fragments of a native protein can be made by enzymatic digestion (e.g., using a restriction enzyme) or chemical degradation of the full length native gene, mRNA or cDNA, or variants of the foregoing.

Nucleic acids that hybridize under stringent conditions to the nucleic acids of SEQ. ID. No. 1, SEQ. ID. No. 2, or SEQ. ID. No. 3, or the complements thereof, can also be used in the invention. Nucleic acids that hybridize to SEQ. ID. No. 1, SEQ. ID. No. 2, or SEQ. ID. No. 3 under low stringency conditions, moderate stringency conditions, or high stringency conditions are within the invention. Other nucleotides within the invention are polynucleotides that share, at least 65% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity to SEQ. ID. No. 1, SEQ. ID. No. 2, or SEQ. ID. No. 3. Nucleic acids that hybridize under stringent conditions to or share at least 65% sequence identity with SEQ, ID. No. 1, SEQ. ID. No. 2, or SEQ. ID. No. 3 can be obtained by techniques known in the art such as by making mutations in the native gene, or by isolation from an organism expressing such a nucleic acid (e.g., an allelic variant).

Nucleic acid molecules encoding fusion proteins are also within the invention. Such nucleic acids can be made by preparing a construct (e.g., an expression vector) that expresses a desired fusion protein when introduced into a suitable host. For example, such a construct can be made by ligating a first polynucleotide encoding a first protein fused in frame with a second polynucleotide encoding a second protein such that expression of the construct in a suitable expression system yields a fusion protein.

The nucleic acid molecules of the invention can be modified at a base moiety, sugar moiety, or the phosphate backbone, e.g., to improve stability of the molecule, hybridization, and the like. For example the nucleic acid molecules of the invention can be conjugated to groups such as peptides (e.g., for targeting host cell receptors in vivo), or agents facilitating transport across the cell membrane (see, e.g., Letsinger et al. 1989; Lemaitre et al., 1987; Tullis R H, PCT Publication No. WO 88/09810, published Dec. 15, 1988), hybridization-triggered cleavage agents. (See, e.g., van der Krol et al. 1988) or intercalating agents (see, e.g., Zon, 1988).

### Antisense, Ribozyme, Triplex Techniques

Another aspect of the invention relates to the use of purified antisense nucleic acids to inhibit expression of proteins involved in ceramide biosynthesis. Antisense nucleic acid molecules within the invention are those that specifically hybridize (e.g., hind) under cellular conditions to cellular mRNA and/or genomic. DNA encoding such proteins in a manner that inhibits expression of the protein, e.g., by inhibiting transcription and/or translation. The binding may be by conventional base pair complementarity, or, for example, in the case of binding to DNA duplexes, through specific interactions in the major groove of the double helix.

Antisense constructs can be delivered, for example, as an expression plasmid which, when transcribed in the cell, produces RNA which is complementary to at least a unique portion of the cellular mRNA which encodes a selected protein involved in the biosynthesis of ceramide. Alternatively, the antisense construct can take the form of an oligonucleotide probe generated ex vivo which, when introduced into target protein expressing cell, causes inhibition of target protein expression by hybridizing with an mRNA and/or genomic sequences coding for the target protein. Such oligonucleotide probes are preferably modified oligonucleotides that are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see, e.g., U.S. Pat. Nos. 5,176,996; 5,264,564; and 5,256,775). Additionally, general approaches to constructing oligomers useful in antisense therapy have been reviewed, for example, by Van der Krol et al. 1988; and Stein et al. 1988. With respect to antisense DNA, oligodeoxyribonucleotides derived from the translation initiation site, e.g., between the −10 and +10 regions of a target protein encoding nucleotide sequence, are preferred.

Antisense approaches involve the design of oligonucleotides (either DNA or RNA) that are complementary to a target mRNA. The antisense oligonucleotides will bind to target mRNA transcripts and prevent translation. Absolute complementarily, although preferred, is not required. The ability to hybridize will depend on both the degree of complementarity and the length of the antisense nucleic acid. Generally, the longer the hybridizing nucleic acid, the more base mismatches with an RNA it may contain and still form a stable duplex or triplex. One skilled in the art can ascertain a tolerable degree of mismatch by use of standard procedures to determine the melting point of the hybridized complex. Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. (Wagner, R W. 1994). Therefore, oligonucleotides complementary to either the 5′ or 3′ untranslated, non-coding regions of a target gene could be used in an antisense approach to inhibit translation of endogenous target mRNA. Oligonucleotides complementary to the 5′ untranslated region of the mRNA should preferably include the complement of the AUG start codon. Although antisense oligonucleotides complementary to mRNA coding regions are generally less efficient inhibitors of translation, these could still be used in the invention. Whether designed to hybridize to the 5′, 3′ or coding region of the target mRNA, preferred antisense nucleic acids are less that about 100 (e.g., less than about 30, 25, 20, or 18) nucleotides in length. Generally, in order to be effective, the antisense oligonucleotide should be 18 or more nucleotides in length, but may be shorter depending on the conditions.

Specific antisense oligonucleotides can be tested for effectiveness using in vitro studies to assess the ability of the antisense oligonucleotide to inhibit gene expression. Preferably such studies (1) utilize controls (e.g., a non-antisense oligonucleotide of the same size as the antisense oligonucleotide) to distinguish between antisense gene inhibition and nonspecific biological effects of oligonucleotides, and (2) compare levels of the target RNA or protein with that of an internal control RNA or protein.

Antisense oligonucleotides of the invention may include at least one modified base or sugar moiety such as those provided above. Antisense oligonucleotides within the invention might also be an alpha-anomeric oligonucleotide. See, Gautier et al. 1987. For example, the antisense oligonucleotide can be a 2′-O-methylribonucleotide (Inoue et al. 1987A), or a chimeric RNA-DNA analogue (Inoue et al. 1987B).

Oligonucleotides of the invention may be synthesized by standard methods known in the art, e.g., by use of an automated DNA synthesizer, as described herein. Phosphorothioate oligonucleotides may be synthesized by the method of Stein et al. 1988. Methylphosphonate oligonucleotides can be prepared by use of controlled pore glass polymer supports (e.g., as described in Sarin et al. 1988).

The invention also provides a method for delivering one or more of the above-described nucleic acid molecules into cells that express the target protein(s). A number of methods have been developed for delivering antisense DNA or RNA into cells. For example, antisense molecules can be introduced directly into a cell by electroporation, liposome-mediated transfection, CaCl-mediated transfection, or using a gene gun. Modified nucleic acid molecules designed to target the desired cells (e.g., antisense oligonucleotides linked to peptides or antibodies that specifically bind receptors or antigens expressed on the target cell surface) can be used. To achieve high intracellular concentrations of antisense oligonucleotides (as may be required to suppress translation on endogenous mRNAs), a preferred approach utilizes a recombinant DNA construct in which the antisense oligonucleotide is placed under the control of a strong promoter (e.g., the CMV promoter).

### Ribozymes

Ribozyme molecules designed to catalytically cleave target mRNA transcripts can also be used to prevent translation of target mRNAs and expression of the target proteins (see, e.g., Wright and Kearney, 2001; Lewin and Hauswirth, 2001; Sarver et al. 1990 and U.S. Pat. No. 5,093,246). As one example, hammerhead ribozymes that cleave mRNAs at locations dictated by flanking regions that form complementary base pairs with the target mRNA might be used so long as the target mRNA has the following common sequence: 5′-UG-3′. See, e.g., Haseloff and Gerlach 1988. As another example, hairpin and hepatitis delta virus ribozymes may also be used. See, e.g., Bartolome et al. 2004. To increase efficiency and minimize the intracellular accumulation of non-functional mRNA transcripts, a ribozyme should be engineered so that the cleavage recognition site is located near the 5′ end of the target RanBP9 mRNA. Ribozymes within the invention can be delivered to a cell using a vector as described below.

Other methods can also be used to reduce target gene expression in a cell. For example, such gene expression can be reduced by inactivating or “knocking out” the target gene or its promoter using targeted homologous reconibination. See, e.g., Ketnpin et al., 1997; Smithies et al. 1985; Thomas and Capecchi 1987 and Thompson et al. 1989. For example, a mutant, non-functional variant of the target gene (or a completely unrelated DNA sequence) flanked by DNA homologous to the endogenous target gene (either the coding regions or regulatory regions of the target gene) can be used, with or without a selectable marker and/or a negative selectable marker, to transfect cells that express the target protein in vivo.

Expression of the target gene might also be reduced by targeting deoxyribonucleotide sequences complementary to the regulatory region of the target gene the promoter and/or enhancers) to form triple helical structures that prevent transcription of the gene in target cells. See generally, Helene, C. 1991; Helene, C., et al. 1992; and Maher, L. J. 3rd 1992. Nucleic acid molecules to be used in this technique are preferably single stranded and composed of deoxyribonucleotides. The base composition of these oligonucleotides should be selected to promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, e.g., containing a stretch of G residues. These molecules will form a triple helix with a DNA duplex that is rich in GC pairs, in which the majority of the purine residues are located on a single strand of the targeted duplex, resulting in CGC triplets across the three strands in the triplex. The potential sequences that can be targeted for triple helix formation may be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizable stretch of either purines or pyrimidines to be present on one strand of a duplex.

The antisense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramide chemical synthesis, RNA molecules may be generated by in vitro and in vivo transcription of DNA sequences encoding the antisense RNA molecule. Such DNA sequences may be incorporated into a wide variety of vectors which incorporate suitable RNA polymerase promoters. Alternatively, antisense cDNA constructs that synthesize antisense RNA constitutively or inducibly, depending on the promoter used, can be introduced stably into cell lines.

dsRNA Agents that Inhibit Ceramide Biosynthesis

RNA interference (RNAi) can be used to decrease the levels of ceramide or inhibit ceramide biosynthesis. RNAi methods can utilize double stranded RNAs, for example, small interfering RNAs (siRNA), short hairpin RNA (shRNA), and micro RNAs (miRNA). The following discussion will focus on dsRNA generally, but one skilled in the art will recognize that many approaches including those discussed below are available for siRNA, shRNA, miRNA and other RNAi molecules.

dsRNA molecules may be designed and/or optimized based upon G/C content at the termini of the dsRNAs, Tₘ of specific internal domains of the dsRNA, dsRNA length, position of the target sequence within the CDS (coding region), and nucleotide content of the 3′ overhangs.

Administration of dsRNA molecules specific for functional target protein, and/or other related molecules with similar functions, can effect the RNAi-mediated degradation of the target mRNA. For example, a therapeutically effective amount of dsRNA specific for serine palmitoyltransferase (SPT) can be adminstered to patient in need thereof at a therapeutically effective time with respect to administration of an opioid to treat or inhibit the development of opioid tolerance, Any nucleotide that effects a decrease in ceramide biosynthesis can be useful in this aspect of the present invention.

Generally, an effective amount of dsRNA molecule can comprise an intercellular concentration from about 1 nanomolar (nM) to about 100 mM, and in various aspects from about 2 nM to about 50 nM, and in other aspects from about 2.5 nM to about 10 nM. It is contemplated that greater or lesser amounts of dsRNA can be administered.

The dsRNA may be administered to the subject by any means suitable for delivering the RNAi molecules to the cells of interest. For example, dsRNA molecules can be administered by gene gun, electroporation, or by other suitable parenteral or enteral administration routes, such as intravenous injection. RNAi molecules can also be administered locally (lung tissue) or systemically (circulatory system) via pulmonary delivery. A variety of pulmonary delivery devices can be effective at delivering functional RanBP9-specific RNAi molecules to a subject. RNAi molecules can be used in conjunction with a variety of delivery and targeting systems, as described in further detail below. For example, dsRNA can be encapsulated into targeted polymeric delivery systems designed to promote payload internalization.

The dsRNA may be targeted to any stretch of less than 30 contiguous nucleotides, generally about 19-25 contiguous nucleotides, in the desired mRNA target sequences. Searches of the human genome database (BLAST) may be carried out to ensure that selected dsRNA sequence will not target other gene transcripts. Thus, the sense strand of the present dsRNA can comprise a nucleotide sequence identical to any contiguous stretch of about 19 to about 25 nucleotides in the target mRNA of the functional target protein (or related molecule with similar function). Generally, a target sequence on the target mRNA can be selected from a given cDNA sequence corresponding to the target mRNA, for example, beginning 50 to 100 nt downstream (i.e., in the 3′ direction) from the start codon. The target sequence can, however, be located in the 5′ or 3′ untranslated regions, or in the region nearby the start codon.

The dsRNA of the invention can comprise an RNA strand (the antisense strand) having a region which is less than 30 nucleotides in length, generally 19-25 nucleotides in length, and is substantially complementary to at least part of an mRNA transcript of a target gene. The use of these dsRNAs enables the targeted degradation of mRNAs of genes that are involved in ceramide biosynthesis. Using cell-based and animal assays, very low dosages of these dsRNA can specifically and efficiently mediate RNAi, resulting in significant inhibition of expression of a target gene. Thus, the methods and compositions of the invention comprising these dsRNAs are useful for treating pathological processes mediated by expression of the target gene, and subsequent ceramide biosynthesis, e.g. opioid tolerance, nitroxidative stress, and neuroimmune activation, by targeting a gene involved in protein synthesis.

The pharmaceutical compositions of the invention comprise a dsRNA having an antisense strand comprising a region of complementarity which is less than 30 nucleotides in length, generally 19-25 nucleotides in length, and is substantially complementary to at least part of an RNA transcript of a target gene, together with a pharmaceutically acceptable carrier.

Accordingly, certain aspects of the invention provide pharmaceutical compositions comprising the dsRNA of the invention together with a pharmaceutically acceptable carrier, methods of using the compositions to inhibit expression of a target gene, and methods of using the pharmaceutical compositions to treat diseases caused by expression of a target gene.

One aspect of the present invention provides dsRNA molecules for inhibiting the expression of a target gene in a cell or mammal, wherein the dsRNA comprises an antisense strand comprising a region of complementarity which is complementary to at least a part of an mRNA formed in the expression of the target gene and wherein the region of complementarity is less than 30 nucleotides in length, generally 19-25 nucleotides in length.

In various aspects of the present invention, the dsRNA can have at least 5, at least 10, at least 15, at least 18, or at least 20 contiguous nucleotides per strand in common with at least one strand, and in various aspects both strands, of various positions within a target sequence.

The dsRNA comprises two RNA strands that are complementary to hybridize to form a duplex structure. One strand of the dsRNA (the antisense strand) comprises a region of complementarity, that is substantially complementary, and generally fully complementary, to a target sequence, derived from the sequence of an mRNA formed during the expression of a target gene, the other strand (the sense strand) comprises a region which is complementary to the antisense strand, such that the two strands hybridize and form a duplex structure when combined under suitable conditions. Generally, the sense and antisense strands of the duplex structure may each comprise from about 15 to about 30, more generally from about 18 to about 25, yet more generally from about 19 to about 24, and most generally from about 19 to about 21 contiguous base pairs in length. Similarly, the region of complementarity to the target sequence may be from about 15 to about 30, more generally from about 18 to about 25, yet more generally from about 19 to about 24, and most generally from about 19 to about 21 contiguous nucleotides in length. The dsRNA of the invention may further comprise one or more single-stranded nucleotide overhang(s). For example, deoxyribonucleotide sequence “tt” or ribonucleotide sequence “UU” can be connected to the 3′-end of both sense and antisense strands to form overhangs. The dsRNA can be synthesized by standard methods known in the art as further discussed below, e.g., by use of an automated DNA synthesizer, such as are commercially available from, for example, Biosearch, Applied Biosystems, Inc. In one aspect of the present invention, a target gene can be a human gene.

In various aspects, the dsRNA comprises at least two sequences selected from this group, wherein one of the at least two sequences is complementary to another of the at least two sequences, and one of the at least two sequences is substantially complementary to a sequence of an mRNA generated in the expression of a target gene.

dsRNAs comprising a duplex structure of between 20 and 23, but specifically 21, base pairs may be particularly effective in inducing RNA interference, however, shorter or longer dsRNAs may be effective as well.

The substantially complementary antisense strand of the dsRNA of the invention may contain one to three mismatches to the target sequence. If the antisense strand of the dsRNA contains mismatches to a target sequence, it is preferable that the area of mismatch not be located in the center of the region of complementarity. If the antisense strand of the dsRNA contains mismatches to the target sequence, it is preferable that the mismatch be restricted to 5 nucleotides from either end, for example 5, 4, 3, 2, or 1 nucleotide from either the 5′ or 3′ end of the region of complementarity, and preferably from the 5′-end. For example, for a 23 nucleotide dsRNA strand which is complementary to a region of a target gene, the dsRNA generally does not contain any mismatch within the central 13 nucleotides. In another aspect, the antisense strand of the dsRNA does not contain any mismatch in the region from positions 1, or 2, to positions 9, or 10, of the antisense strand (counting 5′-3). The methods described within the invention can be used to determine whether a dsRNA containing a mismatch to a target sequence is effective in inhibiting the expression of a target gene. Consideration of the efficacy of dsRNAs with mismatches in inhibiting expression of a target gene is important, especially if the particular region of complementarity in a target gene is known to have polymorphic sequence variation within the population.

In one aspect, a east one end of the dsRNA has a single-stranded nucleotide overhang of 1 to 4, generally 1 or 2 nucleotides. dsRNAs having at least one nucleotide overhang have unexpectedly superior inhibitory properties than their blunt-ended counterparts. Moreover, the presence of only one nucleotide overhang strengthens the interference activity of the dsRNA, without affecting its overall stability. dsRNA having only one overhang has proven particularly stable and effective in vivo, as well as in a variety of cells, cell culture mediums, blood, and serum. Generally, the single-stranded overhang is located at the 3′-terminal end of the antisense strand or, alternatively, at the 3′-terminal end of the sense strand. The dsRNA may also have a blunt end, generally located at the 5′-end of the antisense strand. Such dsRNAs have improved stability and inhibitory activity, thus allowing administration at low dosages, i.e., less than 5 mg/kg body weight of the recipient per day. Generally, the antisense strand of the dsRNA has a nucleotide overhang at the 3′-end, and the 5′-end is blunt. In another aspect, one or more of the nucleotides in the overhang is replaced with a nucleoside thiophosphate.

Exemplary RNA sequences may be targeted to sequences encoding ceramide biosynthesis enzymes including sphingomyelinase, serine palmitoyltransferase, 3-ketosphinganine reductase, ceramide synthase and dihydroceramide desaturase. Examples of mRNA targets are shown in Table 4 below:

TABLE 4
Ceramide Biosynthetic Enzyme Target mRNAs

| | | | | |
|---|---|---|---|---|
| | | GenBank | Protein SEQ | Nucleic Acid |
| Gene Name | Species | Accession No. | ID NO: | SEQ ID NO: |

Sphingomyelinase Enzymes

| | | | | |
|---|---|---|---|---|
| sphingomyelin phosphodiesterase | Homo | NM_000543 | 1 | 2 |
| 1, acid lysosomal | sapiens | | | |
| sphingomyelin phosphodiesterase | Homo | AJ222801 | 3 | 4 |
| 2, neutral membrane | sapiens | | | |
| sphingomyelin phosphodiesterase | Homo | NM_018667 | 5 | 6 |
| 3, neutral membrane | sapiens | | | |
| sphingomyelin phosphodiesterase, | Homo | NM_006714 | 7 | 8 |
| acid-like 3A | sapiens | | | |
| sphingomyelin phosphodiesterase, | Homo | NM_014474 | 9 | 10 |
| acid-like 3B | sapiens | | | |
| sphingomyelin phosphodiesterase | Homo | NM_017751 | 11 | 12 |
| 4, neutral membrane, transcript | sapiens | | | |
| variant 1 | | | | |

Serine Palmitoyltransferase Enzymes

| | | | | |
|---|---|---|---|---|
| serine palmitoyltransferase, long | Homo | NM_006415 | 13 | 14 |
| chain base subunit 1 | sapiens | | | |
| serine palmitoyltransferase, long | Homo | NM_004863 | 15 | 16 |
| chain base subunit 2 | sapiens | | | |
| serine palmitoyltransferase, long | Homo | NM_018327 | 17 | 18 |
| chain base subunit 3 | sapiens | | | |

3-Ketosphinganine Reductase Enzyme

| | | | | |
|---|---|---|---|---|
| 3-ketodihydrosphingosine | Homo | NM_002035.2 | 19 | 20 |
| reductase | sapiens | | | |

Ceramide Synthase Enzymes

| | | | | |
|---|---|---|---|---|
| ceramide synthase 1 | Homo | AF105005 | 21 | 22 |
| | sapiens | | | |
| ceramide synthase 2 | Homo | NM_022075 | 23 | 24 |
| | sapiens | | | |
| ceramide synthase 3 | Homo | NM_178842 | 25 | 26 |
| | sapiens | | | |
| ceramide synthase 4 | Homo | NM_024552 | 77 | 28 |
| | sapiens | | | |
| ceramide synthase 5 | Homo | NM_147190 | 29 | 30 |
| | sapiens | | | |
| ceramide synthase 6 | Homo | NM_203463 | 31 | 32 |
| | sapiens | | | |

Dihydroceramide Desaturase Enzymes

| | | | | |
|---|---|---|---|---|
| sphingolipid delta-4 desaturase 1 | Homo | AF002668 | 33 | 34 |
| | sapiens | | | |
| sphingolipid delta-4 desaturase 2 | Homo | NM_206918 | 35 | 36 |
| | sapiens | | | |

### Pharmacogenomics

Agents, or modulators that have a stimulatory or inhibitory effect on ceramide activity or biosynthesis, as identified by a screening assay can be administered to individuals to treat disorders. In conjunction with such treatment, the pharmacogenomics (i.e., the study of the relationship between a subject's genotype and the subject's response to a foreign modality, such as a food, compound or drug) may be considered. Metabolic differences of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Pharmacogenomics can further be used to determine appropriate dosages and therapeutic regimens. Accordingly, the activity of ceramide, biosynthesis of ceramide, expression of nucleic acids involved in the biosynthetic pathways for ceramide, or mutations in said nucleic acids, in an individual can be determined to guide the selection of appropriate agent(s) for therapeutic or prophylactic treatment.

The activity of ceramide, biosynthesis of ceramide, or expression of nucleic acids involved in a ceramide biosynthetic pathway, or mutations thereof in an individual can be determined to select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a ceramide biosynthesis inhibitor, or a downstream inhibitor of the action or effects of ceramide.

### Pharmaceutical Preparations and Methods of Administration

The identified compositions treat, inhibit, control and/or prevent, or at least partially arrest or partially prevent ceramide biosynthesis and biological conditions that are mediated by ceramide. Such compositions can be administered to a subject at therapeutically effective doses for the inhibition, prevention, prophylaxis or therapy for such illnesses as opioid antinociceptive tolerance, nitroxidative stress, neuroimmune activation, and other conditions mediated by ceramide biosynthesis. The compositions of the present invention comprise a therapeutically effective dosage of a ceramide biosynthesis inhibitor, a term which includes therapeutically, inhibitory, preventive and prophylactically effective doses of the compositions of the present invention and is more particularly defined below. The subject is preferably an animal, including, but not limited to, mammals, reptiles and avians, more preferably horses, cows, dogs, cats, sheep, pigs, and chickens, and most preferably humans.

### Therapeutically Effective Dosage

Toxicity and therapeutic efficacy of such compositions can be determined by standard pharmaceutical procedures in cell cultures or experimental animals for determining the LD₅₀, (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀. Compositions that exhibit large therapeutic indices are preferred. While compositions exhibiting toxic side effects may be used, care should be taken to design a delivery system that targets such compositions to the site affected by the disease or disorder in order to minimize potential damage to unaffected cells and reduce side effects.

The data obtained from the cell culture assays and animal studies can be used in formulating a range of dosages for use in humans and other mammals. The dosage of such compositions lies preferably within a range of circulating plasma or other bodily fluid concentrations that include the ED₅₀ with little or no toxicity. The dosage may vary within this range depending upon the dosage form employed and the route of administration utilized. For any composition of the invention, the therapeutically effective dose can be estimated initially from cell culture assays. A dosage may be formulated in animal models to achieve a circulating plasma concentration range that includes the IC₅₀ (the concentration of the test composition that achieves a half-maximal inhibition of symptoms) as determined in cell culture. Such information can be used to more accurately determine useful dosages in humans and other mammals. Composition levels in plasma may be measured, for example, by high performance liquid chromatography.

The amount of a composition that may be combined with pharmaceutically acceptable carriers to produce a single dosage form will vary depending upon the host treated and the particular mode of administration. It will be appreciated by those skilled in the art that the unit content of a composition contained in an individual dose of each dosage form need not in itself constitute a therapeutically effective amount, as the necessary therapeutically effective amount could be reached by administration of a number of individual doses. The selection of dosage depends upon the dosage form utilized, the condition being treated, and the pa cu ar purpose to be achieved according to the determination of those skilled in the art.

The dosage regime for treating a disease or condition with the compositions and/or composition combinations of this invention is selected in accordance with a variety of factors, including the type, age, weight, sex, diet and medical condition of the patient, the route of administration, pharmacological considerations such as activity, efficacy, pharmacokinetic and toxicology profiles of the particular composition employed, whether a composition delivery system is utilized and whether the composition is administered as a pro-drug or part of a drug combination. Thus, the dosage regime actually employed may vary widely from subject to subject.

### Formulations and Use

The compositions of the present invention may be formulated by known methods for administration to a subject using several routes which include, but are not limited to, parenteral, oral, topical, intradermal, intramuscular, intraperitoneal, intravenous, subcutaneous, intranasal, epidural, and ophthalmic routes. The individual compositions may also be administered in combination with one or more additional compositions of the present invention and/or together with other biologically active or biologically inert agents (“composition combinations”). Such biologically active or inert agents may be in fluid or mechanical communication with the composition(s) or attached to the composition(s) by ionic, covalent, Van der Waals, hydrophobic, hydrophillic or other physical forces. It is preferred that administration is localized in a subject, but administration may also be systemic.

The compositions or composition combinations may be formulated by any conventional manner using one or more pharmaceutically acceptable carriers and/or excipients. Thus, the compositions and their pharmaceutically acceptable salts and solvates may be specifically formulated for administration, e.g., by inhalation or insufflation (either through the mouth or the nose) or oral, buccal, parenteral or rectal administration. The composition or composition combinations may take the form of charged, neutral and/or other pharmaceutically acceptable salt forms. Examples of pharmaceutically acceptable carriers include, but are not limited to, those described in REMINGTON'S PHARMACEUTICAL SCIENCES (A.R. Gennaro, Ed.), 20th edition, Williams & Wilkins PA, USA (2000).

The compositions may also take the form of solutions, suspensions, emulsions, tablets, pills, capsules, powders, controlled- or sustained-release formulations and the like. Such compositions will contain a therapeutically effective amount of the composition, preferably in purified form, together with a suitable amount of carrier so as to provide the form for proper administration to the patient. The formulation should suit the mode of administration.

### Parenteral Administration

The composition or composition combination may be formulated for parenteral administration by injection, e.g., by bolus injection or continuous infusion. Formulations for injection may be presented in unit dosage form in ampoules or in multi-dose containers with an optional preservative added. The parenteral preparation can be enclosed in ampoules, disposable syringes or multiple dose vials made of glass, plastic or the like. The composition may take such forms as suspensions, solutions or emulsions in oily or aqueous vehicles, and may contain formulatory agents such as suspending, stabilizing and/or dispersing agents.

For example, a parenteral preparation may be a sterile injectable solution or suspension in a nontoxic parenterally acceptable diluent or solvent (e.g., as a solution in 1,3-butanediol). Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid may be used in the parenteral preparation.

Alternatively, the composition may be in powder form for constitution with a suitable vehicle, such as sterile pyrogen-free water, before use. For example, a composition suitable for parenteral administration may comprise a sterile isotonic saline solution containing between 0.1 percent and 90 percent weight per volume of the composition or composition combination. By way of example, a solution may contain from about 5 percent to about 20 percent, more preferably from about 5 percent to about 17 percent, more preferably from about 8 to about 14 percent, and still more preferably about 10 percent of the composition. The solution or powder preparation may also include a solubilizing agent and a local anesthetic such as lignocaine to ease pain at the site of the injection. Other methods of parenteral delivery of compositions will be known to the skilled artisan and are within the scope of the invention.

### Oral Administration

For oral administration, the composition or composition combination may take the form of tablets or capsules prepared by conventional means with pharmaceutically acceptable excipients such as binding agents, fillers, lubricants and disintegrants:

### A. Binding Agents

Binding agents include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof. Suitable forms of microcrystalline cellulose include, for example, the materials sold AVICEL-PH-101, AVICEL-PH-103 and AVICEL-PH-105 (available from FMC Corporation, American Viscose Division, Avicel Sales, Marcus Hook, Pa., USA). An exemplary suitable binder is a mixture of microcrystalline cellulose and sodium carboxymethyl cellulose sold as AVICEL RC-581 by FMC Corporation.

### B. Fillers

Fillers include, but are not limited to, talc, calcium carbonate (e.g., granules or powder), lactose, microcrystalline cellulose, powdered cellulose, dextrates, mannitol, silicic acid, sorbitol, starch, pre-gelatinized starch, and mixtures thereof.

### C. Lubricants

Lubricants include, but are not limited to, calcium stearate, magnesium stearate, mineral oil, light mineral oil, glycerin, sorbitol, mannitol, polyethylene glycol, other glycols, stearic acid, sodium tauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, and mixtures thereof. Additional lubricants include, for example, amyloid silica gel (AEROSIL 200, manufactured by W.R. Grace Co. of Baltimore, Md., USA), a coagulated aerosol of synthetic silica (marketed by Deaussa Co. of Plano, Tex., USA), CAB-O-SIL (a pyrogenic silicon dioxide product sold by Cabot Co, of Boston, Mass., USA), and mixtures thereof.

### D. Disintegrants

Disintegrants include, but are not limited to, agar-agar, alginic acid, calcium carbonate, microcrystal line cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

The tablets or capsules may optionally be coated by methods well known in the art. If binders and/or fillers are used with the compositions of the invention, they are typically formulated as about 50 to about 99 weight percent of the composition. Preferably, about 0.5 to about 15 weight percent of disintegrant, preferably about 1 to about 5 weight percent of disintegrant, may be used in the composition. A lubricant may optionally be added, typically in an amount of less than about 1 weight percent of the composition. Techniques and pharmaceutically acceptable additives for making solid oral dosage forms are described in Marshall, SOLID ORAL DOSAGE FORMS, Modern Pharmaceutics (Banker and Rhodes, Eds.), 7:359-427 (1979). Other less typical formulations are known in the art.

Liquid preparations for oral administration may take the form of solutions, syrups or suspensions. Alternatively, the liquid preparations may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and/or preservatives (e.g., methyl or propyl-p-hydroxybenzoates or sorbic acid). The preparations may also contain buffer salts, flavoring, coloring, perfuming and sweetening agents as appropriate. Preparations for oral administration may also be formulated to achieve controlled release of the composition. Oral formulations preferably contain 10% to 95% composition. In addition, the compositions of the present invention may be formulated for buccal administration in the form of tablets or lozenges formulated in a conventional manner. Other methods of oral delivery of compositions will be known to the skilled artisan and are within the scope of the invention.

### Controlled-Release Administration

Controlled-release (or sustained-release) preparations may be formulated to extend the activity of the composition or composition combination and reduce dosage frequency. Controlled-release preparations can also be used to effect the time of onset of action or other characteristics, such as blood levels of the composition, and consequently affect the occurrence of side effects.

Controlled-release preparations may be designed to initially release an amount of a composition that produces the desired therapeutic effect, and gradually and continually release other amounts of the composition to maintain the level of therapeutic effect over an extended period of time. In order to maintain a near-constant level of a composition in the body, the composition could be released from the dosage form at a rate that will replace the amount of composition being metabolized and/or excreted from the body. The controlled-release of a composition may be stimulated by various inducers, e.g., change in pH, change in temperature, enzymes, water, or other physiological conditions or molecules.

Controlled-release systems may include, for example, an infusion pump which may be used to administer the composition in a manner similar to that used for delivering insulin or chemotherapy to specific organs or tumors. Typically, using such a system, the composition is administered in combination with a biodegradable, biocompatible polymeric implant that releases the composition over a controlled period of time at a selected site. Examples of polymeric materials include polyanhydrides, polyorthoesters, polyglycolic acid, polylactic acid, polyethylene vinyl acetate, and copolymers and blends thereof. In addition, a controlled release system can be placed in proximity of a therapeutic target, thus requiring only a fraction of a systemic dosage.

The compositions of the invention may be administered by other controlled-release means or delivery devices that are well known to those of ordinary skill in the art. These include, for example, hydropropylmethyl cellulose, other polymer matrices, gels, permeable membranes, osmotic systems, multilayer coatings, microparticles, liposomes, microspheres, or the like, or a combination of any of the above to provide the desired release profile in varying proportions. Other methods of controlled-release delivery of compositions will be known to the skilled artisan and are within the scope of the invention.

### Inhalation Administration

The composition or composition combination may also be administered directly to the lung by inhalation. For administration by inhalation, a composition may be conveniently delivered to the lung by a number of different devices. For example, a Metered Dose Inhaler (“MDI”) which utilizes canisters that contain a suitable low boiling point propellant, e.g., dichlorodifluoromethane, trichlorofluoromethane, dichlorotetraftuoroethane, carbon dioxide or other suitable gas may be used to deliver a composition directly to the lung. MDI devices are available from a number of suppliers such as 3M Corporation, Aventis, Boehringer Ingleheim, Forest Laboratories, Glaxo-Wellcome, Schering Plough and Vectura.

Alternatively, a Dry Powder inhaler (DPI) device may be used to administer a composition to the lung, DPI devices typically use a mechanism such as a burst of gas to create a cloud of dry powder inside a container, which may then be inhaled by the patient. DPI devices are also well known in the art and may be purchased from a number of vendors which include, for example, Fisons, Glaxo-Wellcome, Inhale Therapeutic Systems, ML Laboratories, Qdose and Vectura. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura. For example, capsules and cartridges of gelatin for use in an inhaler or insufflator may be formulated containing a powder mix of the compound and a suitable powder base such as lactose or starch for these systems.

Another type of device that may be used to deliver a composition to the lung is a liquid spray device supplied, for example, by Aradigm Corporation. Liquid spray systems use extremely small nozzle holes to aerosolize liquid composition formulations that may then be directly inhaled into the lung. For example, a nebulizer device may be used to deliver a composition to the lung. Nebulizers create aerosols from liquid composition formulations by using, for example, ultrasonic energy to form fine particles that may be readily inhaled. Examples of nebulizers include devices supplied by Sheffield/Systemic Pulmonary Delivery Ltd., Aventis and Batelle Pulmonary Therapeutics.

In another example, an electrohydrodynamic (“EHD”) aerosol device may be used to deliver a composition to the lung. EHD aerosol devices use electrical energy to aerosolize liquid composition solutions or suspensions. The electrochemical properties of the composition formulation are important parameters to optimize when delivering this composition to the lung with an EHD aerosol device. Such optimization is routinely performed by one of skill in the art. Other methods of intra-pulmonary delivery of compositions will be known to the skilled artisan and are within the scope of the invention.

Liquid composition formulations suitable for use with nebulizers and liquid spray devices and EHD aerosol devices wilt typically include the composition with a pharmaceutically acceptable carrier. In one exemplary embodiment, the pharmaceutically acceptable carrier is a liquid such as alcohol, water, polyethylene glycol or a perfluorocarbon. Optionally, another material may be added to alter the aerosol properties of the solution or suspension of the composition. For example, this material may be a liquid such as an alcohol, polyglycol or a fatty acid. Other methods of formulating liquid composition solutions or suspensions suitable for use in aerosol devices are known to those of skill in the art.

### Depot Administration

The composition or composition combination may also be formulated as a depot preparation. Such long-acting formulations may be administered by implantation (e.g., subcutaneously or intramuscularly) or by intramuscular injection. Accordingly, the compositions may be formulated with suitable polymeric or hydrophobic materials such as an emulsion in an acceptable oil or ion exchange resins, or as sparingly soluble derivatives such as a sparingly soluble salt. Other methods of depot delivery of compositions will be known to the skilled artisan and are within the scope of the invention.

### Topical Administration

For topical application, the composition or composition combination may be combined with a carrier so that an effective dosage is delivered, based on the desired activity ranging from an effective dosage, for example, of 1.0 μM to 1.0 mM. In one embodiment, a topical composition is applied to the skin. The carrier may be in the form of, for example, and not by way of limitation, an ointment, cream, gel, paste, foam, aerosol, suppository, pad or gelled stick.

A topical formulation may also consist of a therapeutically effective amount of the composition in an ophthalmologically acceptable excipient such as buffered saline, mineral oil, vegetable oils such as corn or arachis oil, petroleum jelly, Miglyol 182, alcohol solutions, or liposomes or liposome-like products. Any of these compositions may also include preservatives, antioxidants, antibiotics, immunosuppressants, and other biologically or pharmaceutically effective agents which do not exert a detrimental effect on the composition. Other methods of topical delivery of compositions will be known to the skilled artisan and are within the scope of the invention.

### Suppository Administration

The composition or composition combination may also be formulated in rectal formulations such as suppositories or retention enemas containing conventional suppository bases such as cocoa butter or other glycerides and binders and carriers such as triglycerides, microcrystalline cellulose, gum tragacanth or gelatin. Suppositories may contain the composition in the range of 0.5% to 10% by weight. Other methods of suppository delivery of compositions will be known to the skilled artisan and are within the scope of the invention.

### Other Systems of Administration

Various other delivery systems are known in the art and can be used to administer the compositions of the invention. Moreover, these and other delivery systems may be combined and/or modified to optimize the administration of the compositions of the present invention.

### Active Ingredient Kits

In various embodiments, the present invention can also involve kits. Such kits can include the compositions of the present invention and, in certain embodiments, instructions for administration. When supplied as a kit, the different components of the composition can be packaged in separate containers and admixed immediately before use. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components. In addition, if more than one route of administration is intended or more than one schedule for administration is intended, the different components can be packaged separately and not mixed prior to use. In various embodiments, the different components can be packaged in one composition for administration together.

Kits may also include reagents in separate containers such as, for example, sterile water or saline to be added to a lyophilized active component packaged separately. For example, sealed glass ampules may contain lyophilized phosphatases and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

### Biological Methods

Methods described above involving conventional molecular biology techniques are generally known in the art and are described in detail in methodology treatises such as MOLECULAR CLONING: A LABORATORY MANUAL, 2nd ed., vol. 1-3, ed. Sambrook et al., Cold Spring Harbor Laboratory Press, Cold Spring Harbor, N.Y., 1989; and CURRENT PROTOCOLS IN MOLECULAR BIOLOGY, ed. Ausubel et al., Greene Publishing and Wiley-Interscience, New York, 1992 (with periodic updates), Various techniques using polymerase chain reaction (PCR) are described, e.g., in Innis et al., PCR PROTOCOLS: A GUIDE TO METHODS AND APPLICATIONS, Academic Tress San Diego, 1990. PCR-primer pairs can be derived from known sequences by known techniques such as using computer programs intended for that purpose. The Reverse Transcriptase Polymerase Chain Reaction (RT-PCR) method used to identify and amplify certain polynucleotide sequences within the invention may be performed as described in Elek et al., in vivo, 14:172-182, 2000). Methods and apparatus for chemical synthesis of nucleic acids are provided in several commercial embodiments, e.g., those provided by Applied Biosystems, Foster City, Calif., and Sigma-Genosys, The Woodlands, Tex. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., GENE THERAPY: PRINCIPLES AND APPLICATIONS, ed. T. Blackenstein, Springer Verlag, 1999; GENE THERAPY PROTOCOLS (METHODS IN MOLECULAR MEDICINE), ed. P. D. Robbins, Humana Press, 1997; and RETRO-VECTORS FOR HUMAN GENE THERAPY, ed. C. P. Hodgson, Springer Verlag, 1996.

### EXAMPLES

Aspects of the present teachings may be further understood in light of the following examples, which should not be construed as limiting the scope of the present teachings in any way.

### Examples 1 and 2

### General Methods

For Examples 1 and 2, below, the following general methods were employed:

### Induction of Morphine-Induced Antinociceptive Tolerance in Mice

Nociceptive thresholds were determined by measuring the latencies of mice placed in a transparent glass cylinder on a hot plate (Ugo Basile, Italy) maintained at 52° C. Determination of antinociception was assessed between 7:00 and 10:00 A.M. Responses indicative of nociception included intermittent lifting and/or licking of the hindpaws, or escape behavior. A cut-off latency of twenty seconds was employed to prevent tissue damage and the results were expressed as Hot Plate Latency Changes (response latency-baseline latency, in seconds). Baseline values ranged between six to eight seconds. Hot plate latencies were taken in mice from all groups on day five before (baseline latency) and forty minutes after (response latency) an acute dose of morphine (3 mg/kg, given subcutaneously), a time previously identified to produce near-to-maximal antinociceptive effect (99±2% antinociceptive effect, n=8).

Mice were injected subcutaneously twice a day (at approximately 7 A.M. and 4 P.M.) with morphine (2×10 mg/kg/day; Mor group) or an equivalent volume of saline (0.1 ml, Control group) over four days, Fumonisin B1 (FB1, 1 mg/kg/day), a competitive and reversible inhibitor of ceramide synthase (Cayman Chemical, Ann Arbor, Mich.), myriocin, an inhibitor of serine palmitosyltransferase, D609, an inhibitor of the acid sphingomyelinase, or their vehicle (saline, 0.1 ml) were given by daily intraperitoneal (i.p.) injection fifteen minutes before each morphine dose (Mori-Drug group). On day five, mice received the first dose of FB1, myriocin, D609, or their respective vehicle, followed fifteen minutes later by the acute dose of morphine. In order to exclude a potential interaction between these interventional drugs and acute morphine, mice were treated as in the Control group, except in the presence of the drug under investigation (Control+Drug). On day five, spinal cord tissues from the lumbar enlargement segment of the spinal cord (L4-L6) and dorsal horn tissues were removed and the tissues processed for immunohistochemical, Western blot, and biochemical analysis as described in the General Methods section. For biochemical determinations of ceramide, the dorsal horn of the spinal cord lumbar segments were harvested and detected by mass spectrometry using electrospray ionization (ESI-MS/MS) and a triple quadrupole mass detector (Han et al., 2005). The spinal cord dorsal horn was sampled because the immunohistochemical staining showed that increases in ceramide were presented primarily in this region. Tolerance to the antinociceptive effect of morphine was indicated by a significant (P<0.05) reduction in Hot Plate Latency Change(seq) after challenge with the acute dose. The percent maximal possible antinociceptive effect (% MPE) was calculated as follows: (response latency-baseline latency)/(cut off latency-baseline latency)×100. Six mice per group were used and all experiments were conducted with the experimenters blinded to treatment conditions. Statistical analysis was performed by one-way ANOVA, followed by multiple Student-Newman-Keuts post hoc tests.

### Light Microscopy

Spinal cord tissues (L4-L6 area) were taken on day five after morphine treatment, Tissue segments were fixed in 4% (w/v) PBS-buffered paraformaldehyde and 7 μm sections were prepared from paraffin embedded tissues. Tissue transversal sections were deparaffinized with xylene, stained with Haematoxylin/Eosin (H&E) and studied using light microscopy (Dialux 22 Leitz) in order to study the superficial laminae of the dorsal horn.

### Immunohistochemical Localization of Ceramide

After deparaffinization, endogenous peroxidase was quenched with 0.3% (v/v) hydrogen peroxide in 60% (v/v) methanol for thirty minutes. Non-specific adsorption was minimized by incubating the section in 2% (v/v) normal goat serum in PBS for twenty minutes. Endogenous biotin or avidin binding sites were blocked by sequential incubation for fifteen minutes with biotin and avidin (DBA), respectively. Sections were incubated overnight with anti-ceramide antibody (1:50 in PBS, v/v, Sigma). Sections were washed with PBS, and incubated with secondary antibody. The counter-stain was developed with a biotin-conjugated goat anti-rabbit IgG and avidin-biotin peroxidase complex (DBA brown color) and nuclear fast red (red background). Positive staining was also stained in brown. To verify the binding specificity for ceramide, some sections were also incubated with only the primary antibody (no secondary antibody) or with only the secondary antibody (no primary antibody). Under these conditions, no positive staining was found in the sections, indicating that the immunoreactions were positive in all of the experiments.

### Tissue Preparation and Lipid Analysis by ESI-MS/MS

Dorsal horn tissues from the lumbar enlargement of spinal cords (50 mg wet weight) were snap frozen and then extracted by the Bligh-Dyer technique (Bligh et al., 1959) in the presence of 1 mg 17:0 ceramide internal standard. Lipid extracts were back-washed with artificial upper phase and then dried under nitrogen prior to storage in 250 ml chloroform under nitrogen until ESI-MS analyses. 50 ml of lumbar spinal cord lipid extract was mixed with 200 ml of methanol containing 10 mM NaOH prior to direct infusion into the ESI source at a flow rate of 3 ml/min as described by others (Han et al., 2005). Ceramides were directly analyzed in the negative-ion mode and detected using tandem mass spectrometry with a collision energy of 32 eV and a collision gas pressure of 2.5 mTorr (argon). With tandem mass spectrometry, ceramides were detected by the neutral loss of m/z 256.2. Typically, a five to ten minute period of signal averaging for each tandem mass spectrum of a lipid extract in the profile mode was employed. Ceramide molecular species were directly quantitated by comparisons of ion peak intensities with that of internal standard (i.e., 17:0 ceramide) after correction for 13C isotope effects.

### Example 1

### Inhibition of Ceramide Biosynthesis Blocks Morphine Tolerance

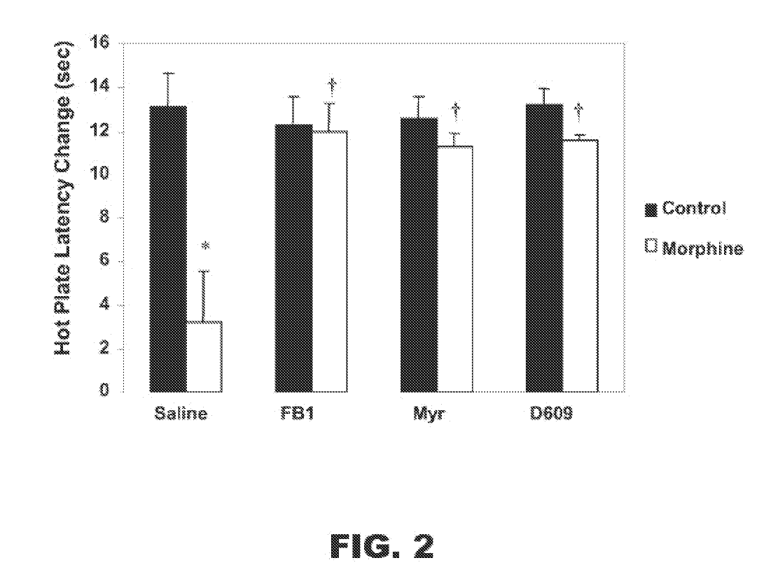

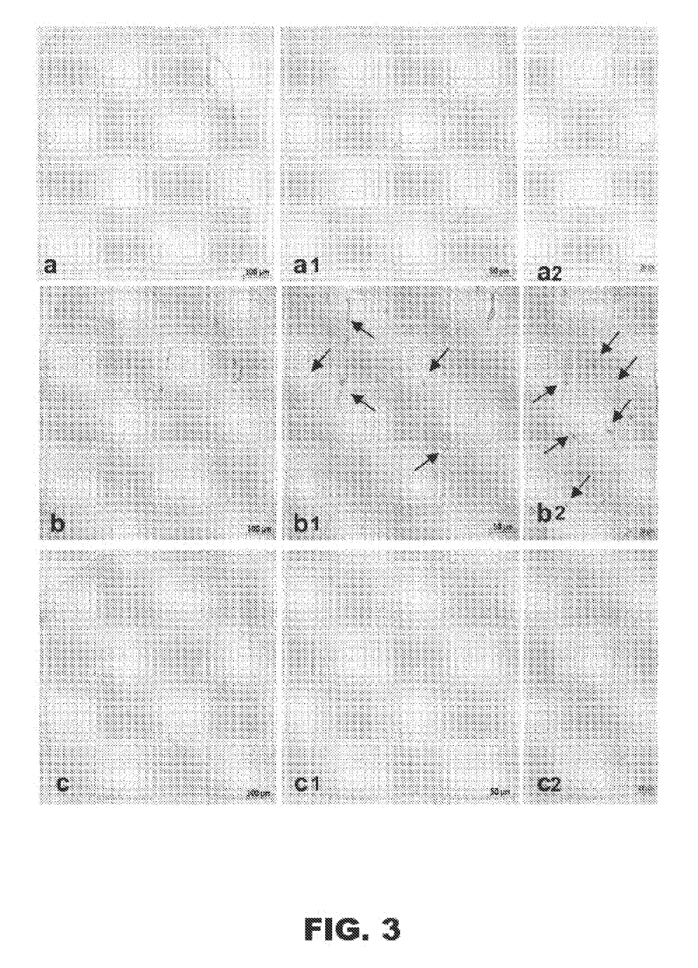

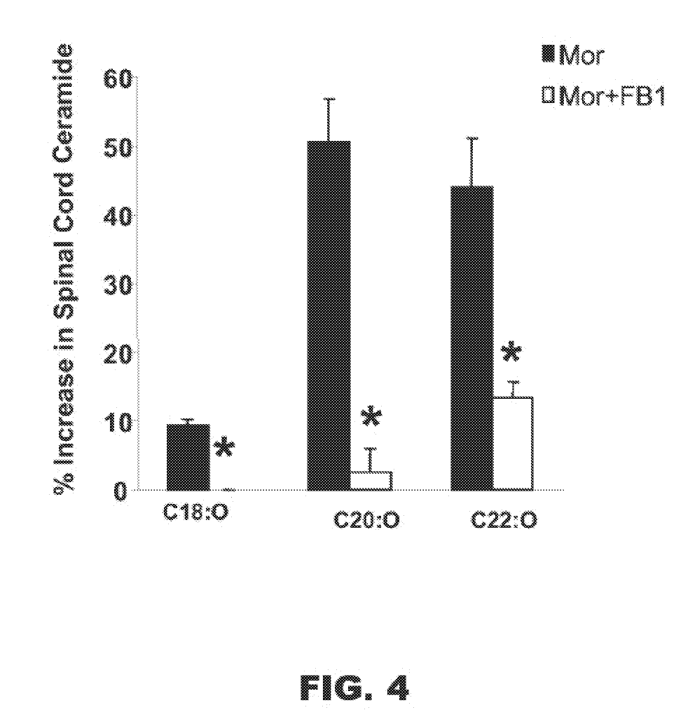

Repeated administration of morphine over four days led to the development of antinociceptive tolerance (FIG. 2; from 93±8 to 20±14% MPE for acute morphine in Control versus Morphine groups respectively (P<0.05)). This was associated with the appearance of ceramide in the superficial layers of the dorsal horn as detected by immunohistochemistry using an anti-ceramide monoclonal antibody (FIG. 3). As shown by ESI-MS/MS, the predominant ceramide species found to be increased by repeated morphine administration in dorsal horn tissues included 18:0, 20:0, and 22:0 ceramide (FIG. 4; n=3). No staining of ceramide was present in the ventral horn.

Co-administration of morphine with FB1 (1 mg/kg) prevented the development of antinociceptive tolerance and the increase in ceramide as measured by immunohistochemical analysis and ESI-MS/MS (FIGS. 3 and 4). To address the potential lack of specificity inherent to pharmacological inhibitors such as FB1, the upstream enzyme in the de novo pathway, serine palmitoyltransferase, was inhibited with myriocin. Similar to FB1, co-administration of morphine with myriocin (0.2 mg/kg) blocked antinociceptive tolerance (FIG. 2). In order to determine whether activation of the acid sphingomyelinase contributed to the development of antinociceptive tolerance, morphine was co-administered with D609 (40 mg/kg). D609 blocked antinociceptive tolerance, as shown in FIG. 2. Since D609 has been reported to inhibit ceramide formation also by inhibiting sphingomyelin synthase (the enzyme that generates sphingomyelin, the substrate for SMAse), it is possible that inhibition of both enzymes accounts for the overall beneficial action of D609 (Schutze et al., 1992; Luberto et al., 1998). Collectively, these results implicate the participation of the de novo and the sphingomyelin pathways in ceramide biosynthesis (FIG. 1).

The inhibitory effects of these drugs were not attributable to acute antinociceptive interactions with morphine since the responses to acute morphine in the control groups and control groups treated with FB1, myriocin or D609 were similar (FIG. 2). When tested alone these drugs had no antinociceptive effects (not shown).

### Example 2

### Induction of Antinociceptive/Analgesic Tolerance in Mice Following Subcutaneous Chronic Delivery of Morphine by Osmotic Minipumps

Antinociceptive/analgesic tolerance was also induced in mice using a continuous infusion of morphine with osmotic minipumps as previously described (Vera-Portocarrero et al., 2006). Thus, the experimental protocol is more clinically relevant than the one using repeated bolus injections. Furthermore, an osmotic pump ensures continuous delivery of morphine without intermittent periods of withdrawal. To this end, pilot testing was performed examining the effects of FB1 in this dosing paradigm. Morphine (50 mg/kg, Morphine groups) or saline (Control groups) was administered to male CD-1 mice using osmotic minipumps implanted subcutaneously to deliver morphine over seven days. A total of four groups n=6 mice/group) were used. FB1 (1 mg/kg/day), or an equivalent volume of its vehicle, was given together with morphine by i.p injection once a day for six days. On day six, thirty minutes after the injection of FB1, acute nociception was determined by the tail flick test (Ugo Basile, Italy), with baseline latencies of four to five seconds and a cutoff time of ten seconds. Latencies were taken in all animals before and thirty minutes after the acute challenge dose of morphine given by intraperitoneal injection (3 mg/kg, i.p) using the tail flick. These time points were chosen because they were identified from previous studies to produce near-to maximal antinociception. When compared to the control group, infusion of morphine led to the development of antinociceptive tolerance, and this was attenuated in mice that received FB1 (from 90±5% to 15±4% MPE tier acute morphine in the control groups and in the Morphine groups respectively, P<0.01; and from 15±4% to 87±4% MPE for acute morphine in the Mor groups and in the Mor+FB1 groups respectively, P<0.01). FB1 did not affect responses to acute morphine (90±5% to 85±6% MPE for acute morphine in the control groups and in the control+FB1 groups respectively).

### Examples 3 through 6

### General Methods

For Examples 3 through 6 below, the following general methods were employed:

### Induction of Morphine-Induced Antinociceptive Tolerance in Mice

Male CD-1 mice (24-30 g; Charles River Laboratory) were housed 5 per cage and maintained under identical conditions of temperature (21±1° C.) and humidity (65%±5%) with a 12-hour light/12-hour dark cycle and allowed food ad libitum. Nociceptive thresholds were determined by measuring latencies (in seconds) of mice placed in a transparent glass cylinder on a hot plate (Ugo Basile, Italy) maintained at 52° C. Determination of antinociception was assessed between 7 and 10:00 A.M. All injections were given intra-peritoneally (i.p.) or subcutaneously (s.c.) in a volume of 0.1 and 0.3 ml, respectively, at approximately 7 A.M. and 4 P.M. Responses indicative of nociception included intermittent lifting and/or licking of the hindpaws or escape behavior. Hot plate latencies were taken in mice from all groups on day five before (baseline latency) and thirty minutes after (response latency) an acute dose of morphine (0.3-3 mg/kg) or its vehicle (saline). Results were expressed as percentage of maximum possible antinociceptive effect, which was calculated as follows: (response latency−baseline latency)/(cut-off latency−baseline latency)×100. A cut-off latency of twenty seconds was employed to prevent tissue damage. Ten mice per group were used and all experiments were conducted with the experimentors blinded to treatment conditions. Fumonisin B1 (FB1), a competitive and reversible inhibitor of ceramide synthase (Delgado et al., 2006), myriocin, an inhibitor of serine palmitoyltransferase (Delgado et al., 2006), D609, an inhibitor of the acid sphingomyelinase (Delgado et al., 2006) or their vehicle (saline) were given by daily i.p. injection fifteen minutes before each dose of morphine. The following experimental groups were used:

Naive (N) Group: In this group, mice were injected twice per day with an i.p. injection of saline (the vehicle used to deliver the drugs to other groups over four days) and a s.c. injection of saline (the vehicle used to deliver morphine over four days). On day five, mice received an i.p. injection of saline followed fifteen minutes later by a s.c. injection of saline.

Naive+Drug Groups: In these groups, mice were injected twice a day for four days with an i.p. injection of the highest dose of FB1 (1 mg/kg/d), myriocin (0.4 mg/kg/d), or D609 (20 mg/kg/d), and an s.c. injection of saline. On day five, mice received an i.p. injection of FB1 (0.5 mg/kg), myriocin (0.2 mg/kg), D609 (10 mg/kg), followed fifteen minutes later by a s.c. injection of saline.

Vehicle (V) Group: In this group, mice were injected twice per day for four days with an i.p, injection of saline and a s.c. injection of saline. On day five, mice received an i.p. injection of saline followed fifteen minutes later by a s.c. injection of acute morphine eliciting near-to-maximal antinociception (3 mg/kg).

Vehicle+Drug Groups: In these groups, were injected twice per day for four days with an i.p. injection of the highest dose of FB1 (1 mg/kg/d), myriocin (0.2 mg/kg/d), or D609 (20 mg/kg), followed fifteen minutes later by s.c. doses of acute morphine giving from ten to ninety-five percent antinociceptive responses within forty minutes of administration (0.1-3 mg/kg).

Morphine (Mor) Group: in this group, mice were injected twice per day for four days with an i.p. injection of saline and a s.c. injection of morphine (20 mg/kg/d). On day five, mice received an i.p. injection of saline followed fifteen minutes by a s.c. dose of acute morphine (3 mg/kg).

Morphine+Drug Groups: In these groups, mice were injected twice per day for four days with an i.p. injection of FB1 (0.25, 0.5, and 1 mg/kg/d), myriocin (0.1, 0.2, and 0.4 mg/kg/d), or D609 (10, 20, and 40 mg/kg/d), and a s.c. injection of morphine (20 mg/kg/d). On day five, mice received an i.p. dose of FB1 (0.5 mg/kg), myriocin (0.2 mg/kg), or D609 (20 mg/kg), followed fifteen minutes later by the s.c. doses of acute morphine (3 mg/kg).

In another set of experiments, and in order to address whether FB1, myriocin, or D609 reverse the expression of tolerance, mice were treated twice a day with morphine as described above and on day five received a single i.p. dose of FB1 (1 mg/kg), myriocin (0.4 mg/kg), D609 (40 mg/kg) followed fifteen minutes later by the acute dose of morphine (3 mg/kg).

On day five after the behavioral tests, spinal cord tissues from the lumbar enlargement segment of the spinal cord (L-6) and dorsal horn tissues were removed and tissues processed for immunohistochemical, Western blot, and biochemical analysis.

Mice were trained before experimentation for their ability to remain for one-hundred twenty seconds on a revolving Rotarod apparatus (accelerating units increase from 3.5 to 35 rpm in five minutes). Mice (n=4 per group) were injected with an i.p. injection of the highest dose of FB1 (1 mg/kg), myriocin (0.4 mg/kg), D609 (40 mg/kg) used to block antinociceptive tolerance or its vehicle. Mice (n=4 per group) were tested and examined for motor implants on the Rotarod at fifteen, thirty, and sixty minutes after drug administration as described in the method section. The latency time to fall off the Rotarod was determined. A cut-off time of one-hundred twenty seconds was used.

### Determination of Ceramide Synthase Activity

About 60 to 80 mg of spinal cord homogenates were incubated with [³H]-palmytic acid (2.5 μCi/ml, GE Healthcare, England) for one hour. Lipids were extracted with ice-cold methanol containing 2% acetic acid and 5% chloroform and resolved using thin-layer chromatography. Lipids co-migrating with standards were scraped and quantified by lipid scintillation counting as described elsewhere (Castillo et al., 2007).

### Determination of Sphingomyelinase Activity

Sphingomyelinase activity was measure using Amplex® Red Sphingomyelinase Assay Kit (Molecular Probes, Eugene, Oreg.) following manufacturer's instructions. First, spinal cord tissues were homogenized in buffers for each specific assay as previously described (Dobrowsky and Kolesnick, 2001). For the acid isoforms, Na acetate (100 mM and pH 5.0) lysis buffer was used. 2 mM EDTA was added to the lysis buffer for detection of the insoluble isoform. For neutral isoform detection, the tissues were homogenized in Hepes (20 mM, pH 7.4) lysis buffer. The kinetics for sphingomyelinase activity was measured in a fluorescence microplate reader for two hours followed by normalization per protein concentration of the sample. Hydrogen peroxide and sphingomyelinase were used as positive controls.

### Determination of Serine Palmitoyl Transferase (SPT) Activity

SPT activity was determined by measuring the incorporation of [³H] serine into 3-ketosphinganine following the method previously described (Williams et al., 1984). The results were normalized by the samples' protein concentration.

### Light Microscopy

Spinal cord tissues (L4-16 area) were taken on day five after morphine treatment. Tissue segments were fixed in 4% (w/v) PBS-buffered paraformaldehyde and 7 μm sections were prepared from paraffin embedded tissues. Tissue transversal sections were deparaffinized with xylene, stained with Haematoxylin/Eosin (H&E) and studied using light microscopy (Dialux 22 Leitz) in order to study the superficial laminae of the dorsal horn.

### Immunohistochemistry for Ceramide, GFAP, and Iba1

For ceramide staining, endogenous peroxidase was quenched with 0.3% (v/v) hydrogen peroxide in 60% (v/v) methanol for thirty minutes after deparaffinization. Non-specific adsorption was minimized by incubating the section in 2% (v/v) normal goat serum in PBS for twenty minutes. Endogenous biotin or avidin binding sites were blocked by sequential incubation for fifteen minutes with biotin and avidin (DBA), respectively. Sections were incubated overnight with anti-ceramide antibody (1:50 in PBS, v/v Sigma). Sections were then washed with PBS, and incubated with secondary antibody. The counter stain was developed with a biotin-conjugated goat anti-rabbit IgG and avidin-biotin peroxidase complex (DBA brown color) and nuclear fast red (red background). Positive staining was detected as a brown color. To verify the binding specificity thr ceramide, some sections were also incubated with only the primary antibody (no secondary antibody) or with only the secondary antibody (no primary antibody). Under these conditions, no positive staining was found in the sections, indicating that the immunoreactions were positive in all of the experiments. For GFAP and IB1a staining, frozen sections were used. Mice were anesthetized with halothane (Sigma, St. Louis, Mo.) and intracardially perfused with a fresh solution of 4% paraformaldehyde in phosphate buffer (PB) (0.1 M sodium phosphate, pH 7.4). After perfusion, the spinal cord lumbar enlargement was quickly removed and postfixed in the same fixative overnight. Tissues were then immersed in a solution of 30% (w/v) sucrose in PB at 4° C. until the tissues were processed for sectioning. Transverse spinal sections (20 μm) were cut in a cryostat and mounted on polylysine-coated slides and processed for immunohistochemistry. All of the sections were blocked with 2% goat serum in 0.3% Triton X-100 for one hour at room temperature (RT). For immunofluorescent staining, the sequential spinal sections were incubated with primary antibody, either polyclonal rabbit anti-GFAP (GFAP, astrocyte marker, 1:500, Dako) or anti-IBa1 (microglia marker, 1:500, Wako Pure Chemical, Osaka Japan) overnight at 4° C., followed by incubation with FITC- (for GFAP) Texas-red- (for IBa1) conjugated secondary antibodies (1:500) for two hours at RT in the dark. After washing, the stained sections were examined with a fluorescence microscope (Fluovert, Leitz, Germany) and images were captured with a Sony DX500 digital camera (Sony, Tokyo, Japan). All images were taken at the same exposure settings. To determine the specificity of immunoreaction, the negative control sections were processed as above procedures but omitting the primary antibody.

### Immunoprecipitation and Western Blot

Animals were rapidly sacrificed (<1 min) in a CO₂ chamber and the dorsal portion of the spinal cord lumbar region enlargement removed and stored at −80° C. until used. Cytosolic and nuclear extracts were prepared as previously described (Bethea et al., 1998), with minor modifications. Tissues from each mouse were suspended in extraction Buffer A (0.2 mM PMSF, 0.15 pepstatin A, 20 μM leupeptin, 1 mM sodium orthovanadate), homogenized for two minutes, and centrifuged at 1,000×g for ten minutes at 4° C. Supernatants were collected as the cytosolic fraction. The pellets containing nuclei were re-suspended in Buffer B (1% Triton X-100, 150 mM NaCl, 10 mM TRIS-HCl pH 7.4, 1 mM EGTA, 1 mM EDTA, 0.2 mM PMSF, 20 μm leupeptin, 0.2 mM sodium orthovanadate). After centrifugation for thirty minutes at 15,000×g at 4° C., the supernatants were collected as nuclear extracts and then stored at −800° C. for further analysis. The levels of IκB-α, phospho-NF-κB p65 (serine 536), were quantified in cytosolic fraction from spinal cord tissue, while NF-κB p65 levels were quantified in nuclear fraction. The membranes were blocked with 5% (w/v) non-fat dried milk (PM) in 1×PBS for forty minutes at room temperature and subsequently probed with specific anti-IκB-α (Santa Cruz Biotechnology, 1:1000), phospho-NF-κB p65 (serine 536) (Cell Signaling, 1:1000), GFAP, or Mal with 5% w/v non-fat dried milk in 1×PBS, 0.1% Tween-20 (PMT) at 4° C. overnight, followed by incubations with either peroxidase-conjugated bovine anti-mouse IgG secondary antibody or peroxidase-conjugated goat anti-rabbit IgG (1:2000, Jackson ImmunoResearch, West Grove, Pa.) for one hour at room temperature. Manganese superoxide dismutase (MnSOD) nitration was determined with western blot analysis of immunoprecipitated protein complex in total lysates using antibodies specific to these proteins. The immunoprecipitated proteins were resolved in 12% SUS-PAGE mini and proteins transferred to nitrocellulose membranes. Membranes were blocked for one hour at room temperature (RT) in 1% Bovine Serum Albumin (BSA)/0.1% Thimerosal in 50 mM Tris•HCl, (pH 7.4)/150 mM NaCl/0.01% Tween 20 (TBS/T), then incubated with rabbit polyclonal antibodies for MnSOD (1:2000, Upstate Biotechnology, NY) followed by incubation of secondary antibodies conjugated with peroxidase for one hour at room temperature. Protein bands were visualized by enhanced chemiluminescence (ECL, Amersham Biosciences, Arlington Heights, Ill.). After stripping, all membranes were reprobed with either monoclonal anti-β-actin or α-tubulin antibody (1:20.000; Sigma; St Louis, Mo.) as a loading control. The relative expression of the protein levels as the band density for IκB-α (˜37 kDa), phospho NF-κB (65 kDa), NF-κB p65 (75 kDa), MnSOD (˜29 kDa), GFAP (˜50 kDa), and Iba1(˜17 kDa) was quantified by scanning of the X-ray films with GS-700 Imaging Densitometer (BIO-RAD U.S.A.) and a computer program (Molecular Analyst, IBM).

### Measurement of Mn and CuZn-SOD Activities

Dorsal halves of the spinal cord lumbar region enlargement (L4-L6) were homogenized with 10 mM phosphate buffered saline (pH 7.4) in a Polytron homogenizer and then sonicated on ice for one minute (twenty seconds, three times). The sonicated samples were subsequently centrifuged at 1,100×g for ten minutes. SOD activity was measured in the supernatants as described previously (Wang et al., 2004). A competitive inhibition assay was performed which used xanthine-xanthine oxidase-generated superoxide to reduce nitroblue tetrazolium (NBT) to blue tetrazolium salt. The reaction was performed in sodium carbonate buffer (50 mM, pH 10.1) containing EDTA (0.1 mM), nitroblue tetrazolium (25 μM), xanthine and xanthine-oxidase (0.1 mM and 2 nM respectively; Boehringer, Germany). The rate of NBT reduction was monitored spectrophotometrically (Perkin Elmer Lambda 5 Spectrophotometer, Milan, Italy) at 560 nm. The amount of protein required to inhibit the rate of NTB reduction by 50% was defined as one unit of enzyme activity. Cu/Zn-SOD activity was inhibited by performing the assay in the presence of 2 mM NaCN after pre-incubation for thirty minutes. Enzymatic activity was expressed in units per milligram of protein (Wang et al., 2004).

### Statistics

For paired group analysis, Students t-test was performed. For paired multiple groups, analysis of variance followed by Student-Newman-Keuls test was employed to analyze the data. Results are expressed as mean±SEM for n animals. A statistically significant difference was defined as a P value<0.05.

### Example 3

### Inhibition of Ceramide Biosynthesis Blocks Morphine Antinociceptive

### Tolerance Without Affecting Motor Function

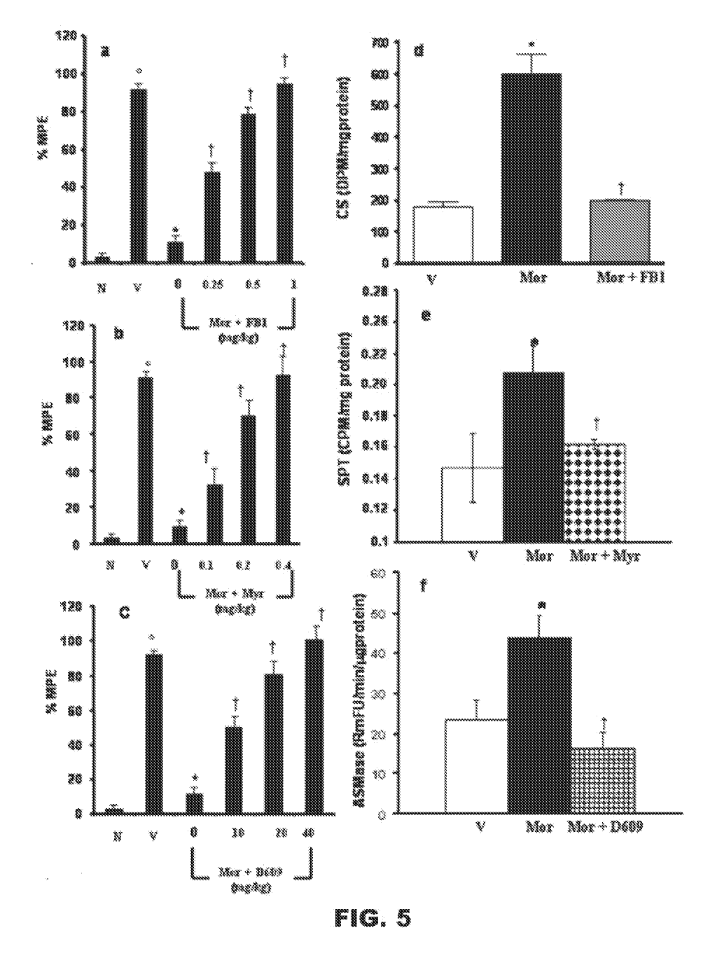

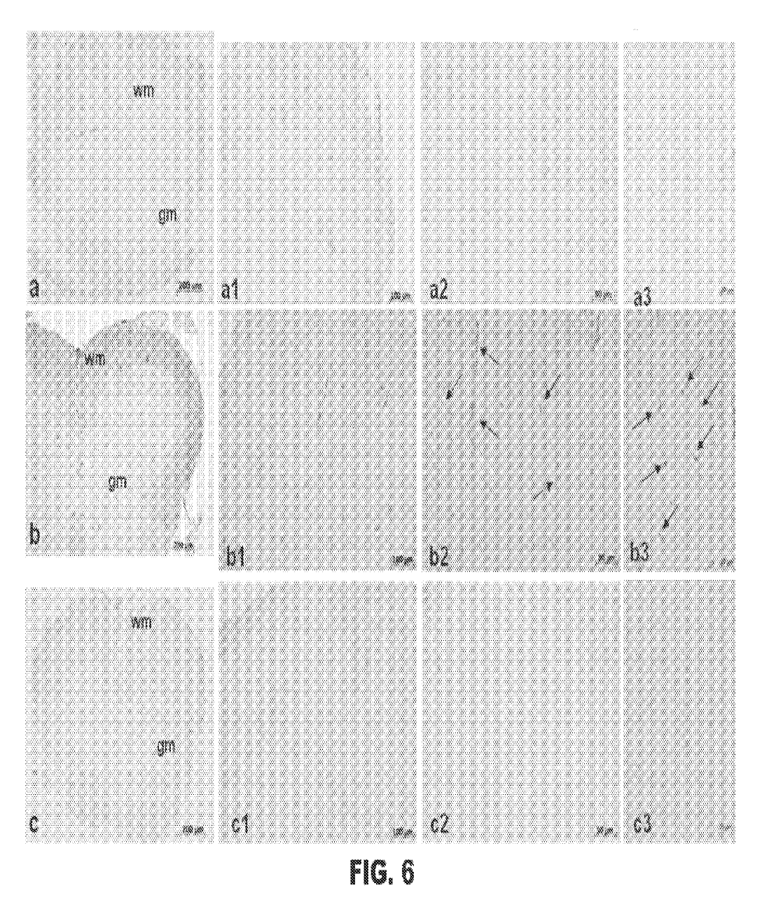

When compared with animals receiving an equivalent injection of saline (naïve group, “N”), acute injection of morphine (3 mg/kg) in animals that received saline over four days (vehicle group, “V”) produced a significant near-maximal antinociceptive response [percent maximal possible antinociceptive effect, (YOMPE, ranging from 90-95%] (FIG. 5a-c). On the other hand, repeated administration of morphine over the same time course (morphine group, “0” Mor+Drug groups) led to the development of antinociceptive tolerance as evidenced by a significant loss of antinociceptive response on the part of animals in the group (FIG. 5a-c). Antinociceptive tolerance was associated with increased enzymatic activity of ceramide synthase (CS, FIG. 5d), serine palmytoyl transferase (SPT, FIG. 5e) and the insoluble form of acid sphingomyelinase (ASMase, FIG. 51), and was also associated with the appearance of ceramide in the superficial layers of the dorsal horn, as detected by immunohistochemistry (arrows, FIG. 6b-b3). Activities of the soluble form of ASMase and the neutral SMase were not changed compared to vehicle (not shown). Baseline latencies in vehicle and morphine groups were statistically insignificant from each other and ranged from six to eight seconds (n=10).

To investigate whether the increased ceramide synthesis had a functional role in the development of morphine's antinociceptive tolerance, morphine was co-administered with specific inhibitors of both de novo and sphingomyelinase pathways, Co-administration of morphine with fumonisin B1 (FB1; 1 mg/kg/d, n=10), a competitive and reversible inhibitor of ceramide synthase (Delgado et al., 2006), attenuated as expected the increase in CS activity (FIG. 5d), and ceramide immunostaining (FIG. 6c-c3). Also attenuated in a dose-dependent manner (0.1-1 mg/kg/d, n=10) was the development of tolerance (FIG. 5a). Similar results were obtained with another inhibitor of the de novo pathway, myriocin, which targets the rate-limiting, most upstream enzyme, serine palmitoyltransferase (Delgado et al., 2006). Indeed, co-administration of morphine with myriocin (0.4 mg/kg/d, n=10) blocked, as expected, the activation of SPT (FIG. 5e), the increase in ceramide immunostaining (not shown), and the development of antinociceptive tolerance in a dose-dependent manner (0.1-0.4 mg/kg/d, n=10) (FIG. 5b). The role of the SMase pathway was determined by treating animals with tricyclodecan-9-xanthogenate (D609; 10-40 mg/kg/d, n=10), an inhibitor of this enzyme (Delgado et al., 2006). When co-administered with morphine, D609 (40 mg/kg/d, 10) blocked the increased activity of ASMase (FIG. 51) and ceramide immunostaining (not shown), and blocked in a dose-dependent manner (10-40 mg/kg/d, n=10 the development of tolerance (FIG. 5c). Since D609's inhibitory activities are not limited to SMase, but may also include sphingomyelin synthase, it is possible that inhibition of both enzymes accounted for the overall beneficial action of D609 against tolerance development.

As can be seen from FIG. 6, no positive staining for ceramide was observed in the dorsal horn when compared to the ventral horn tissues of the control groups (FIG. 6a-a3). Five days after morphine treatment, a marked appearance of positive staining for ceramide (brown) was observed in the dorsal horn when compared to the ventral horn (FIG. 6b-b3, see arrows). FB1 treatment abolished the presence of positive staining for ceramide (FIG. 6c-c3). Tissue sections were stained using 3,3′-diaminobenzidine (DAB). The results shown in FIG. 6 are representative of at least three experiments performed on different days. Tissues from the dorsal and ventral spinal cord were taken on the same day and processed together.

In order to establish whether these inhibitors, when tested at the highest dose shown to block antinociceptive tolerance, cause motor function impairment, mice were treated with myriocin (0.4 mg/kg), FB1 (1 mg/kg) or D609 (40 mg/kg) and then tested on the Rotarod for potential motor function deficits at fifteen, thirty, and sixty minutes after drug administration. When compared to the vehicle-treated group, these drugs did not show signs of Rotarod deficits over the observed time frame (n=4, not shown).

### Example 4

### Inhibition of Ceramide Biosynthesis Does Not Affect the Acute Antinociceptive Effects to Morphine

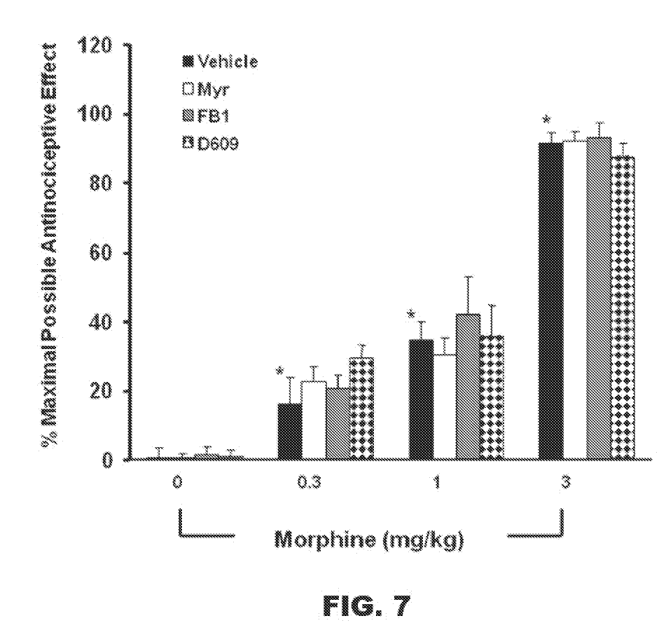

The inhibitory effects of FB1, myriocin, or D609 were not attributable to acute antinociceptive interactions between FB1, myriocin, or D609 and morphine, since the responses to acute morphine (0.3-3 mg/kg, n=10) in animals treated with the highest dose of FB1 (1 mg/kg/d, n=10), myriocin (0.4 mg/kg/d, n=10), D609 (40 mg/kg/d, n=10), or their vehicle over five days was statistically insignificant (FIG. 7). These results suggest that ceramide is not involved in spinal neurotransmission and antinociceptive signaling in response to brief administration of morphine. When tested alone, at the highest dose, none of FB1, myriocin, or D609 had antinociceptive effects. Thus, on day five hot plate latencies following a s.c. injection of saline in the vehicle group, or in animals that received the highest dose of FB1, myriocin, or D609, were statistically insignificant and ranged between six and seven seconds (n=10; data not shown).

### Example 5

### Inhibition of Ceramide Biosynthesis Does Not Reverse Establish Morphine Tolerance

The loss of the antinociceptive effect of morphine observed on day five in the morphine group was not restored by a single administration of the highest dose of FB1 (1 mg/kg, n=6), myriocin (0.4 mg/kg/d, n=6), or D609 (40 mg/kg, n=6) used and given by i.p. injection fifteen minutes before the acute dose of morphine (3 mg/kg). Thus, the % MPE was 96±3%, 10±2%, 7±3%, 13±2% and 11±2% for the vehicle, morphine, morphine plus FB1, morphine plus myriocin, and morphine plus D609 groups respectively (n=6, P<0.5 for all groups). These results suggest that these pharmacological agents inhibit the development of, and not the expression, of tolerance.

The profound and equal inhibitory effect of myriocin, FB1, and D609 indicate that controlling ceramide levels in the dorsal horn of the spinal cord is paramount to preventing antinociceptive tolerance, regardless of the enzymatic pathway by which it is synthesized. Therefore, only FB1 was chosen as an effective and well-characterized inhibitor of ceramide biosynthesis in subsequent mechanistic studies aimed to understanding the downstream pathophysiological effects initiated by an increase in spinal cord ceramide.

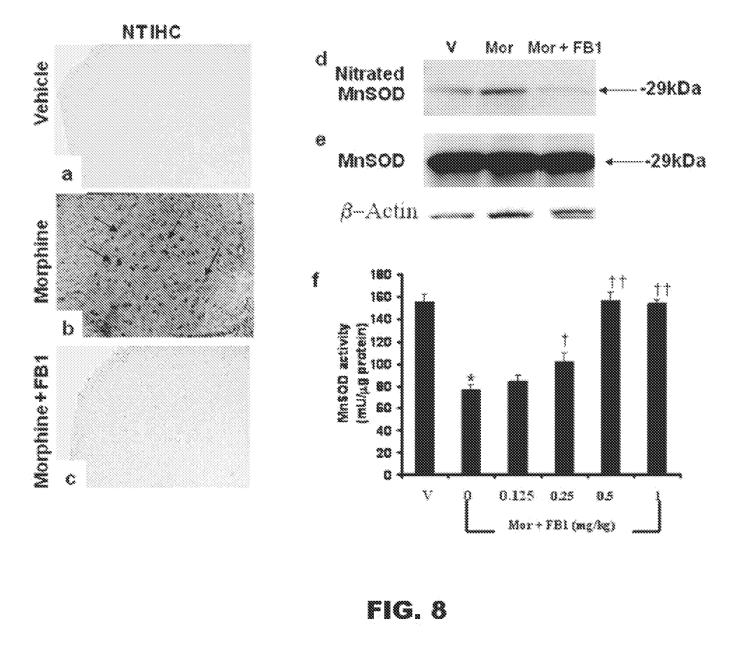

Peroxynitrite is a key player in the development of morphine antinociceptive tolerance, and data shows that formation of 3-nitrotyrosine (NT) in the superficial layers of the dorsal horn during morphine antinociceptive tolerance originates from spinal production of peroxynitrite (Muscoli, 2007). Detection of NT in this setting can therefore be reliably used as marker of peroxynitrite. The inventor of the present invention discovered that the appearance of NT staining in tolerant mice (FIG. 8b) was blocked by co-administration of morphine with FB1 (1 mg/kg/d; FIG. 8c), evidence of the contribution of ceramide in the production of spinal peroxynitrite. Post-translational nitration and enzymatic inactivation of MnSOD in the spinal cord is an important source for sustaining high levels of spinal peroxynitrite during the development of central sensitization associated with morphine antinociceptive tolerance (Muscoli, 2007). As shown in FIG. 8, FB1 (1 mg/kg/d) prevented post-translational nitration of mitochondrial manganese superoxide dismutase (MnSOD) as shown by immunoprecipitation (from 400±50 to 850±70 densitometry units±SEM for vehicle and morphine respectively, n=5, P<0.001; and from 850±70 to 350±45 for morphine and morphine plus FB1 respectively, n=5, P<0.001; a representative gel of five animals is shown in FIG. 8d) and restored in a dose-dependent manner (0.25-1 mg/kg/d, n=5) the loss of its enzymatic activity as measured spectrophotometrically (FIG. 80. Total levels of MnSOD protein did not change among the three groups (a representative gel of five animals is shown in FIG. 8e).

### Example 6

### Inhibition of Ceramide Biosynthesis Attenuates Neuroimmune Activation

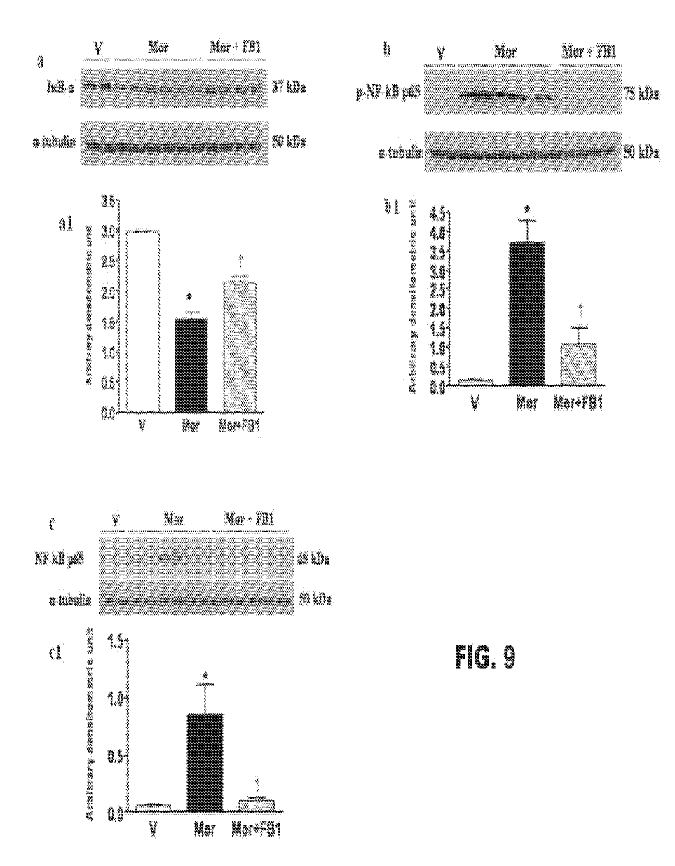

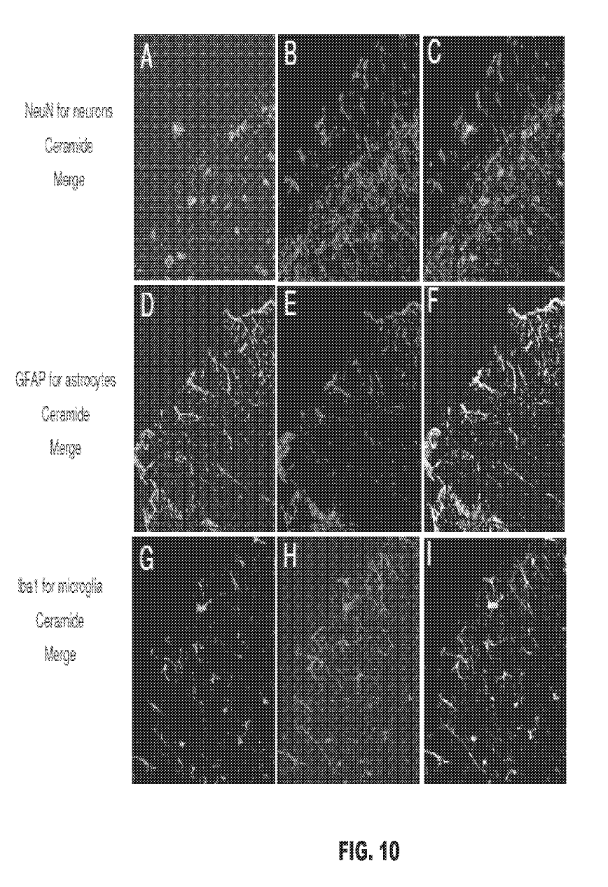

On day five, when compared to the vehicle group, acute injection of morphine (3 mg/kg, n=10) in the morphine group led to a significant activation of NF-κB as demonstrated by IκB-α degradation (FIG. 9a, a1), increased Ser536 phosphorylation (FIG. 9b, b1), and increased total NF-kB p65 nuclear expression (FIG. 9c, c1). The development of morphine antinociceptive tolerance is associated with neuronal activation (FIG. 10a), with activation of astrocytes (FIG. 10d), microglial cells (FIG. 10g) and with the appearance of ceramide (FIG. 10b-h) as detected by immunofluorescence studies (FIG. 10i, f) in dorsal horn tissues of the lumbar portion of the spinal cord. Results show that ceramide preferentially co-localizes with glial cells (astrocytes and microglia, FIG. 10f, i) but not with neurons (FIG. 10c).

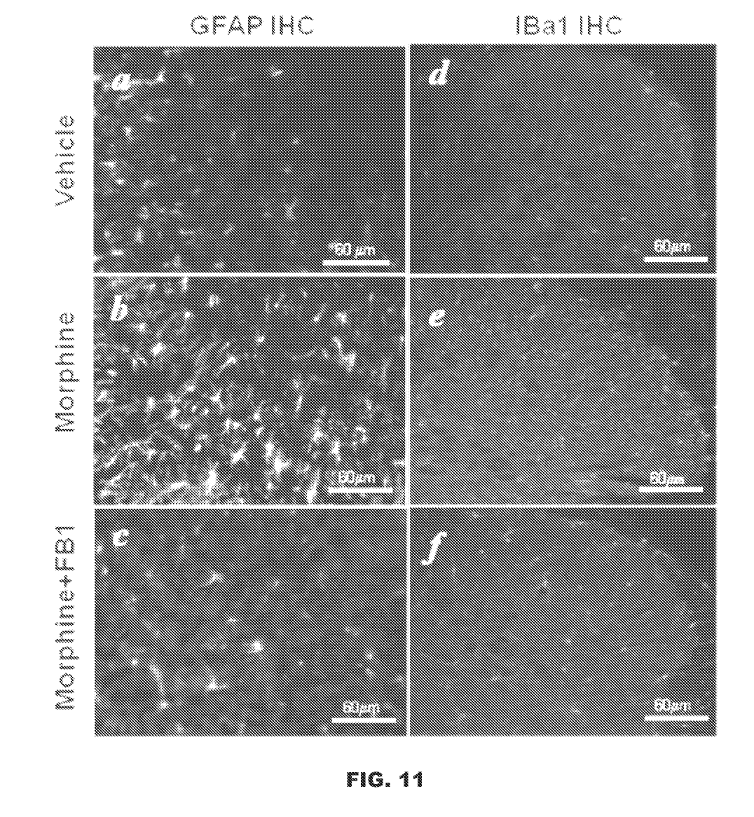

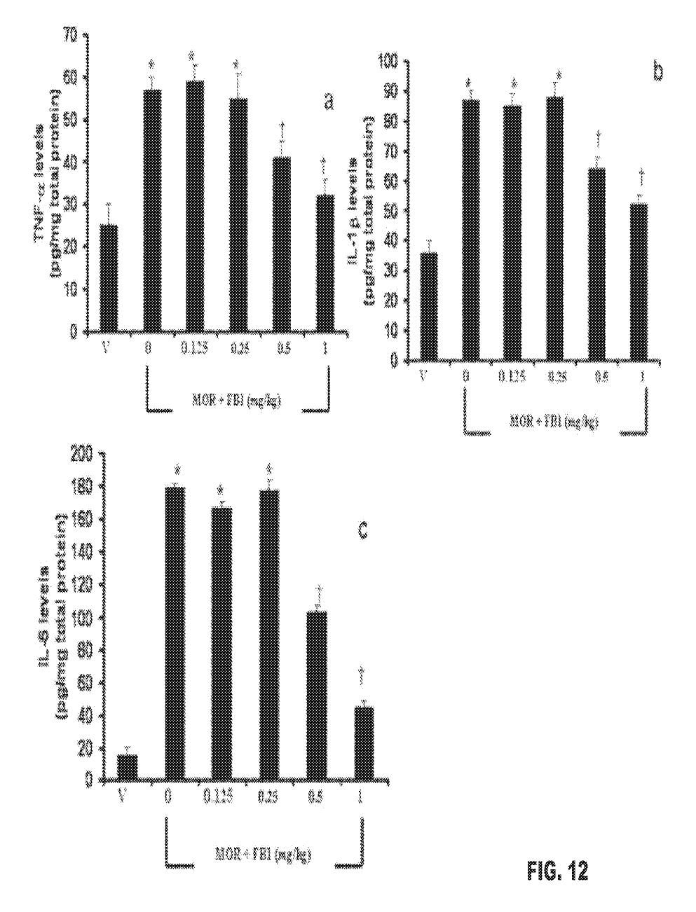

Furthermore, acute injection of morphine in the morphine group increased glial cell activation determined by enhanced spinal expression of GFAP (glial fibrillary acidic protein; a cellular marker for astrocytes; from 5455.13±0.514 to 7343.95±0.527 densitometry units, n=5, P<0.01; FIG. 11b) and IBa1 (ionized calcium binding adaptor molecule 1; a cellular marker for microglia (Narita et al., 2006), from 241.66±0.039 to 541.29±0.073 densitometry units±SEM, n=5, P<0.001; FIG. 11e), measured by immunohistochemistry and western blotting (not shown). Finally, acute injection of morphine in the morphine group increased immunoreactivity for TNF-α, IL-1β and IL-6 in the dorsal horn of the lumbar spinal cord, as measured by ELISA (n=10, FIG. 12a-c). NF-kB activation was attenuated by FB1 (1 mg/kg/d) (FIG. 9a-c), as was the activation of astrocytes (from 7343.95±0.527 to 4627.38±0.483 densitometry units±SEM, n=5, P<0.001; FIG. 11c) and microglial cell (from 541.29±0.073 to 275.53±0.053 densitometry units SEM, n=5, P<0.001; FIG. 110. Fumonisin B1 (0.25-1 mg/kg/d, n=10) reduced in a dose-dependent fashion increased release of TNF-α, IL-1β and IL-6 (FIG. 12a-c).

As shown in FIG. 11, when compared to vehicle (FIG. 11a and 11d), acute administration of morphine in tolerance mice led to neuroimmune activation as evidenced by increased GFAP (a marker of activated astrocytes; FIG. 11b) and Iba1 (a marker of activated microglial cells; FIG. 11e) immunoreactivity in the superficial layers of the dorsal horn, the activation of which was blocked by administration of 1 mg/kg/d of FB1 (FIG. 11c and 11f). Micrographs (×20 magnification) are representative of at least three experiments performed on different animals on different days.

As seen in FIG. 12, on day five, when compared to acute morphine in the vehicle group, repeated administration of morphine over the same time course led to a significant increase in TNF-α, IL-1β, and IL-6 in dorsal horn tissues (FIG. 12a-c), which were reduced by FB1 in a dose-dependent manner (0.25-1 mg/kg/d; n=10; FIG. 12a-c). Results are expressed as mean±SEM for n=10 animals.

The foregoing examples provide a foundation for certain novel findings the inventor has made with respect to the present invention. These findings are set forth now in greater detail.

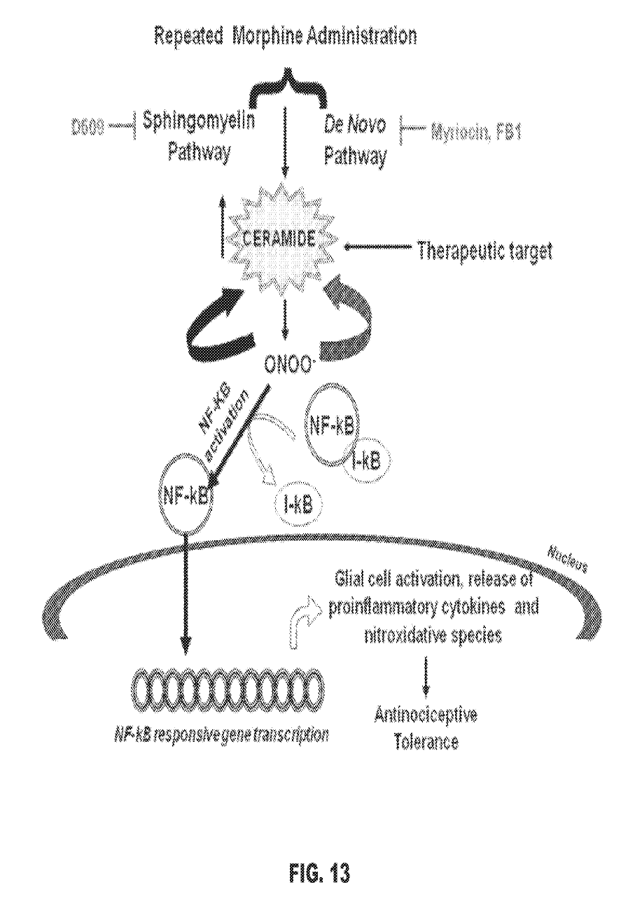

The present inventor has discovered a novel mechanism triggered by repeated administration of morphine that increases the activity of enzymes involved in the biosynthesis of ceramide from both the de novo and sphingomyelinase pathways; pharmacological inhibition of both pathways blocks the development of antinociceptive tolerance (see, for example, FIG. 13). Thus, these enzymatic pathways are functionally responsible for both spinal cord ceramide synthesis and antinociceptive tolerance to morphine. The critical role of ceramide in the control of neural apoptosis has been attributed to its generation through both sphingomyelin hydrolysis by neutral (Brann et al., 2002) and/or acid sphingomyelinases and de novo synthesis (Blazquez et al., 2000). The present discovery that the activity of the soluble and neutral forms of SMAse does not increase in response to repeated morphine administration suggests that either these enzyme isoforms do not contribute to the development of tolerance or that they do but that their enzymatic activity returns to baseline levels by the time of assay. Addressing the relative contributions of each isoform can be done reliably as selective inhibitors are developed. The profound and equal inhibitory effect of the three pharmacological inhibitors myriocin, FB1, and D609 on the antinociceptive tolerance to morphine indicates that controlling ceramide levels in the dorsal horn of the spinal cord is paramount to preventing tolerance, regardless of the enzymatic pathway by which it is synthesized. Therefore, only FB1 was chosen as an effective and well characterized inhibitor of ceramide biosynthesis in subsequent mechanistic studies aimed to understand the downstream pathophysiological effects initiated by an increase in spinal cord ceramide. The upstream events that link repeated morphine administration with the activation of ceramide biosynthesis remain to be elucidated.

The present discoveries implicate ceramide as an upstream signaling mediator in one of two major pathobiochemical mechanisms for development of morphine anti nociceptive tolerance, namely peroxynitrite-mediated nitroxidative stress and neuroimmune activation (see FIG. 13). Considerable evidence implicates peroxynitrite-mediated nitroxidative stress in the development of pain of several etiologies and, importantly, in opiate antinociceptive tolerance, caused by the presence of superoxide (Salvemini, 2001; Muscoli, 2007), nitric oxide (Pasternak, 1995) and more recently peroxynitrite (Muscoli, 2007). Ceramide stimulates the formation of reactive nitroxidative species including superoxide and nitric oxide (Pahan et al., 1998; Goldkorn et 2005). In turn, superoxide, nitric oxide, and peroxynitrite can increase steady-state concentrations of ceramide by activating sphingomyelinases and by increasing the degradation of ceramidases, the enzymes responsible for the degradation of ceramide (Pautz et al., 2002). The foregoing supports the close and reciprocal interaction between the nitroxidative and ceramide metabolic pathways: such close interplay contributes to the overall increase in the levels of ceramide and thus ceramide-mediated damage. The present discovery that inhibition of ceramide biosynthesis blocks peroxynitrite suggests that ceramide is an important signaling event in the formation of peroxynitrite, further supporting the intimate relationship between the ceramide metabolic and the nitroxidative pathways as observed in other pathological settings (Delogu et al., 1999; Kolesnick, 2002; Goggel et al., 2004; Masini et al., 2005; Petrache et al., 2005). A biologically relevant feature of peroxynitrite is post-translational tyrosine nitration and consequent modification of protein function (Radi, 2004) as exemplified by MnSOD, the enzyme that normally keeps concentrations of superoxide under tight control (McCord and Fridovich, 1969).

Peroxynitrite-mediated nitration of MnSOD inactivates the enzyme, leading to an increase in superoxide levels thereby favoring peroxynitrite fbrmation in several disease states (Yamakura et al., 1998; MacMillan-Crow et 2001; Yamakura et al., 2001) including in the development of morphine tolerance (Muscoli, 2007) and hyperalgesia associated with acute inflammation and in response to NMDA-receptor activation (Wang et al., 2004; Muscoli, 2007). As described herein, inhibition of ceramide biosynthesis blocks nitration of MnSOD by attenuating the formation of peroxynitrite, thus restoring the enzymatic activity of this enzyme. FB1 thus interrupts a potentially vicious cycle known to influence the presence of nitroxidative stress.

Neuroimmune activation contributes to morphine antinociceptive tolerance, as shown in both preclinical (Song and Zhao, 2001; Watkins et al., 2007) and clinical studies (Lu et 2004). Thus, anticytokine approaches and/or inhibitors of glial metabolism block morphine-induced hyperalgesia and antinociceptive tolerance (Song and Zhao, 2001; Watkins et al., 2007). Ceramide activates, through mechanisms ill-defined, several redox-sensitive transcription factors, including NF-kB, which in turn regulate the production of many inflammatory and pronociceptive cytokines. Inhibition of ceramide biosynthesis with inhibitors of the sphingomyelinase or de novo pathways blocks NF-kB activation and synthesis of TNF-α, IL-1β and IL-6 in animal models of acute and chronic inflammation (Delogu et al., 1999; Kolesnick, 2002; Gogget et al., 2004; Masini et al., 2005; Petrache et 2005). Described herein is the novel discovery that ceramide acts as a signaling mediator in neuroimmune activation. The present discoveries also suggest that activation of NF-κB is a key step in this process. Indeed, inhibition of ceramide biosynthesis by FB1 prevents NF-κB activation, blocks astrocytic and microglial cell activation, and suppresses the increase in TNF-α, IL-1β and IL-6 in dorsal horn tissues, thereby blocking antinociceptive tolerance. The present discoveries suggest that a mechanism through which ceramide activates NF-κB is via peroxynitrite. This is supported by the fact that 1) inhibition of ceramide biosynthesis blocks spinal formation of peroxynitrite, as demonstrated by the present discoveries; 2) peroxynitrite activates several redox-sensitive transcription factors, including NF kB and AP-1, as well MAPK kinases such as p38 kinase, to release TNF-α, IL-1β and IL-6 (Matata and Galinanes, 2002; Ndengele et al., 2005); and 3) peroxynitrite contributes to the development of antinociceptive tolerance through release of spinal TNF-α, IL-1β and IL-6 (Muscoli, 2007). Importantly, Oat cell activation can generate several nitroxidative species implicated in the development of morphine antinociceptive tolerance, including superoxide (Salvemini, 2001; Muscoli, 2007), nitric oxide (Pasternak, 1995) and peroxynitrite (Muscoli, 2007). It is important to recognize that ceramide is a potent proapoptotic signaling lipid, and that spinal apoptosis has been linked to antinociceptive tolerance (Mayer et al., 1999; Lim et al., 2005). In this context, whether ceramide contributes to the formation of dorsal horn “dark neurons” (Mayer et 1999) observed in antinociceptive tolerance is a viable possibility that needs to be explored in future studies.

The present discoveries have defined for the first time the importance of ceramide in the development of antinociceptive tolerance, and have provided evidence for the contribution of at least two mechanistic pathways through which this sphingolipid exerts its actions, namely peroxynitrite-derived nitroxidative stress and neuroimmune activation (see FIG. 13). These data provide a pharmacological basis for validating the approach of developing inhibitors of the ceramide metabolic pathway as adjuncts to opiates in the management of pain.

### Other Embodiments

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

### REFERENCES CITED

Citation of a reference herein shall not be construed as an admission that such is prior art relevant to patentability of the present invention. Specifically incorporated herein by reference in their entireties are the following publications:
Amer S, Rawal N, Gustafsson L L. Clinical experience of long-term treatment with epidural and intrathecal opioids—a nationwide survey. Acta Anaesthesiol Scand 1988; 32:253-259
Bartolomé J, Castillo I and Carreno V, Ribozymes as antiviral agents, Minerva Med. 2004 95:11-24
Baum, Solid-phase synthesis of benzodiazepines, C&EN, Jan. 18, 1993, page 33.
Bektas M, Jolly P S, Milstien S and Spiegel S., A specific ceramide kinase assay to measure cellular levels of ceramide, Analytical Biochemistry 2003 320:259-265.
Bethea J R, Castro M, Keane R W, Lee T T, Dietrich W D and Yezierski R P, Traumatic spinal cord injury induces nuclear factor-kappaB activation, J Neurosci 1998 18:3251-3260.
Blazquez C, Galve-Roperh I and Guzman M, De novo-synthesized ceramide signals apoptosis in astrocytes via extracellular signal-regulated kinase. Faseb J 2000 14:2315-2322.
Bligh E G, Dyer W J. A rapid method of total lipid extraction and purification. Can J Biochem Physiol 1959; 37:911-917.
Brann A B, Tcherpakov M, Williams I M, Futerman A H and Fainzilber M, Nerve growth factor induced p75-mediated death of cultured hippocampal neurons is age-dependent and transduced through ceramide generated by neutral sphingomyelinase. J Biol Chem 2002 277:9812-9818.
Campbell, D A, and Bermak, J C, Phosphonate ester synthesis using a modified Mitsunobu condensation. J. Org. Chem. 1994, 59:658-660.
Castillo S S, Levy M, Wang C, Thaikoottathil J V, Khan E and Goldkorn T, Nitric oxide-enhanced caspase-3 and acidic sphingomyelinase interaction: a novel mechanism by which airway epithelial cells escape ceramide-induced apoptosis. Exp Cell Res 2007 313:816-823.
Celerier E, Laulin J P, Corcuff J B, Le Moal M, Simonnet G. Progressive enhancement of delayed hyperalgesia induced by repeated heroin administration: a sensitization process. J Neurosci 2001; 21:4074-4080.
Celerier Rivat C, Jun Y, Laulin J P, Larcher A, Reynier P, Simonnet G. Long-lasting hyperalgesia induced by fentanyl in rats: preventive effect of ketamine. Anesthesiology 2000; 92:465-472.
Chen, C., Ahlberg-Randall, L A., Miller, R B., Jones, A D. and. Kurth, M J. ‘Analogous’ Organic synthesis of small-compound libraries: Validation of combinatorial chemistry in small-molecule synthesis, J. Am. Chem. Soc. 1994, 116:2661-2662.
Cho, C Y.; Moran, E J.; Chemy, S R.; Stephans, J C.; Fodor, S P A.; Adams, C L.; Sundaram, A.; Jacobs, J W.; Schultz, P G., An Unnatural Biopolymer, Science 1993, 261:1303-1305
Covino B, Dubner, R, Gybels, J, Losterlitz H W, Liebsking, J C, Sternbach, R A, Vylicky L, Yarnamura, H, Zuiramerman, M., Ethical standards for investigations of experimental pain in animals. PAIN 1980 9:141-143.
Cremesti A E and Fischl A S, Current methods for the identification and quantitation of ceramides: an overview, Lipids. 2000 35:937-945.
Dawkins J L, Hulme D J, Brahmbhatt S B, Auer-Grumbach M and Nicholson G A, Mutations in SPTLC1, encoding serine palmitoyltransfrase, long chain base subunit-1, cause hereditary sensory neuropathy type I. Nat Genet 2001 27:309-312.
De Conno F. Caraceni A, Martini C. Spoldi E, Salvetti M, Ventafridda V. Hyperalgesia and myoclonus with intrathecal infusion of high-dose morphine. Pain 1991; 47:337-339.
Delgado A, Casas J, Llebaria A, Abad J L, Fabrias G, Inhibitors of sphingolipid metabolism enzymes. Biochim Biophys Acta, 2006 December; 1758(12):1957-77.
Delogu G, Famularo G, Amati F, Signore L, Antonucci A, Trinchieri V, Di Marzio L and Cifone M G, Ceramide concentrations in septic patients: a possible marker of multiple organ dysfunction syndrome. Crit Care Med 1999 27:2413-2417.
Devulder J. Hyperalgesia induced by high-dose intrathecal sufentanil in neuropathic pain. J Neurosurg Anesthesiol. 1997; 9:146-148.
Dobrowsky R T and Kolesnick R N, Analysis of sphingomyelin and ceramide levels and the enzymes regulating their metabolism in response to cell stress. Methods Cell Biol 2001 66:135-165.
Erdreich-Epstein A, Tran L B, Cox O T, Huang E Y, Laug W E, Shimada H, Millard M. Endothelial apoptosis induced by inhibition of integrins alphavbeta3 and alphavbeta5 involves ceramide metabolic pathways. Blood 2005; 105:4353-4361.
Foley K M. Misconceptions and controversies regarding the use of opioids in cancer pain. Anticancer Drugs 1995; 6 Suppl 3:4-13.
Furka, A., Sebesty n, F., Asgedom, M. and Dib, G. (1991) General method for rapid synthesis of multicomponent peptide mixtures, Int J Peptide Prot Res 1991, 37; 487-493.
Goggel R, Winoto-Morbach S. Vielhaber G, Imai Y, Lindner K, Brade L, Brade H, Ehlers S, Slutsky A S, Schutze S, Gulbins F and Uhlig S, PAF-mediated pulmonary edema: a new role for acid sphingomyelinase and ceramide. Nat Med 2004 10:155-160.
Goldkorn T, Ravid T and Khan E M, Life and death decisions: ceramide generation and EGF receptor trafficking are modulated by oxidative stress. Antioxid Redox Signal 2005 7:119-128
Gautier C, Morvan F, Rayner B, Huynh-Dinh T, Igolen J, Imbach J L, Paoletti C and Paoletti J., Alpha-DNA. IV: Alpha-anomeric and beta-anomeric tetrathymidylates covalently linked to intercalating oxazolopyridocarbazole. Synthesis, physicochemical properties and poly (rA) binding, Nucleic Acids Res. 1987 15:6625-6641.
Hagihara, M., Anthony, N. J., Stout, T. J., Clardy, J. and Schreiber, Vinylogous Polypeptides: An Alternative Peptide Backbones, J. Amer. Chem. Soc, 1992 114:6568-6570.
Han X, Gross R W. Shotgun lipidomics: electrospray ionization mass spectrometric analysis and quantitation of cellular lipidomes directly from crude extracts of biological samples. Mass Spectrom Rev 2005; 24:367-412.
Hélène C, The anti-gene strategy: control of gene expression by triplex-forming-oligonucleotides, Anticancer Drug Des 1991 6:569-584.
Helene C, Thuong N T and Harel-Bellan A, Control of gene expression by triple helix-forming oligonucleotides. The antigene strategy, Ann N Y Acad Sci 1992 660:27-36
Haseloff J and Gerlach W L, Simple RNA enzymes with new and highly specific endoribonuclease activities, Nature 1988 334:585-591.
Hirschmarm, R, Nicolaou, K C, Pietranico, S., Salvino, J., Leahy, E M., Sprengeler, P A., Furst, G., Strader, C D. and Smith III, A B., Nonpeptidal peptidomimetics with β-D-glucose scaffolding. A partial somatostatin agonist bearing a close structural relationship to a potent, selective substance P antagonist, J. Am. Chem. Soc., 1992, 114; 9217-9218.
Hobbs-Dewitt, S., Kiely, J. S., Stankovic, C. J., Schroeder, M C, Reynolds Cody, D M and Pavia, M R Proc. Natl. Acad. Sci. USA, 1993 90; 6909-6913.
Horinouchi K, Erlich S. Peri. D P, Fertinz K, Bisgaier C L, Sandhoff K, Desnick R J, et al. Acid sphingomyelinase deficient mice: a model of types A and B Niemann-Pick disease. Nat Genet 1995; 10:288-293.
Houghten, R. A., Pinilla, C., Blondelle, S. E., Appel, J. R., Dooley, C. T., & Cuervo, J. H. Generation and use of synthetic peptide combinatorial libraries for basic research and drug discovery, Nature 1991 354; 84-86.
Inoue H, Hayase Y, Imura A, Iwai S, Miura K and Ohtsuka E, Synthesis and hybridization studies on two complementary nona(2′-O-methyl)ribonucleotides, Nucleic Acids Res. 1987A 15:6131-6148.
Inoue H, Hayase Y, Iwai S. Ohtsuka E, Sequence-dependent hydrolysis of RNA using modified oligonucleotide splints and RNase H., FEBS Lett. 1987B 215:327-330
Joseph E K and Levine J D, Caspase signalling in neuropathic and inflammatory pain in the rat. Eur J Neurosci 2004 20:2896-2902.
Kempin S A, Liljegren S J, Block L M, Rounsley S D, Yanofsky M F and Lam E Targeted disruption in Arabidopsis, Nature 1997 389:802-803.
Kolesnick R, The therapeutic potential of modulating the ceramide/sphingomyelin pathway. J Clin Invest 2002 110:3-8.
Lemaitre M, Bayard B and Lebleu B., Specific antiviral activity of a poly(L-lysine)-conjugated oligodeoxyribonucleotide sequence complementary to vesicular stomatitis virus N protein mRNA initiation site, Proc Natl Acad Sci U S A. 1987 84:648-652.
Letsinger R L, Zhang G R, Sun D K, Ikeuchi T and Sarin P S., Cholesteryl-conjugated oligonucleotides: oligonucleotides: synthesis, properties, and activity as inhibitors of replication of human immunodeficiency virus in cell culture, Proc Natl Acad Sci U S A. 1989 86:6553-6556.
Lewin A S and Hauswirth W W, Ribozyme gene therapy: applications for molecular medicine, Trends Mol Med 2001 7:221-228.
Liang R, Yan L, Loebach J, Ge M, Uozumi Y, Sekanina K, Horan N, Gildersleeve J, Thompson C, Smith A, Biswas K. Still W C, and Kahne D., Parallel synthesis and screening of a solid phase carbohydrate library, Science 1996 274:1520-1521
Liebisch G. Drobnik W, Reil M, Triimbach B, Arnecke R, Olgemolier B, Roscher A and Schmitz G., Quantitative measurement of different ceramide species from crude cellular extracts by electrospray ionization tandem mass spectrometry (ESI-MS/MS), The Journal of Lipid Research, 1999 40:1539-1546.
Lim G, Wang S, Lim J A and Mao J, Activity of adenylyl cyclase and protein kinase A contributes to morphine-induced spinal apoptosis. Neurosci Lett 2005 389:104-108.
Lu C H, Chao P C, Borel C O, Yang C P, Yeh C C, Wong C S and Wu C T, Preincisional intravenous pentoxifylline attenuating perioperative cytokine response, reducing morphine consumption, and improving recovery of bowel function in patients undergoing colorectal cancer surgery. Anesth Analg 2004 99:1465-1471; table of contents.
Luberto C, Hannan Y A. Sphingomyelin synthase, a potential regulator of intracellular levels of ceramide and diacylglycerol during SV40 transformation, Does sphingomyelin synthase account for the putative phosphatidylcholine-specific phospholipase C? J Biol Chem 1998; 273:14550-14559,
McCord J M and Fridovich I, Superoxide dismutase. An enzymic function for erythrocuprein (hemocuprein). J Biol Chem 1969 244:6049-6055.
MacMillan-Crow L A, Cruthirds D L, Ahki K M, Sanders P W and Thompson J A, Mitochondrial tyrosine nitration precedes chronic allograft nephropathy. Free Radic Biol Med 2001 31:1603-1608,
Maher L J 3rd, DNA triple-helix formation: an approach to artificial gene repressors? Bioessays 1992 14:807-815.
Masini E, Bani D, Vannacci A, Pierpaoli S, Mannaioni P F, Comhair S A, Xu W, Muscoli C. Erzurum S C and Salvemini D, Reduction of antigen-induced respiratory abnormalities and airway inflammation in sensitized guinea pigs hy a superoxide dismutase mimetic. Free Radic Biol Med 2005 39:520-531.
Mao J, Price D D, Mayer D J. Mechanisms of hyperalgesia and morphine tolerance: a current view of their possible interactions. Pain 1995; 62:259-274.
Matata B M and Galinanes M, Peroxynitrite is an essential component of cytokines production mechanism in human monocytes through modulation of nuclear factor-kappa B DNA binding activity. J Biol Chem 2002 277:2330-2335.
Mayer D J, Mao J, Holt J and Price D D, Cellular mechanisms of neuropathic pain, morphine tolerance, and their interactions, Proc Natl Acad Sci USA 1999 96:7731-7736
Muscoli C, Cuzzocrea S, Ndengele M M, Mollace V, Porreca F, Fabrizin F, Esposito E, Masini M, Matuschak G M, and Salvemini D, Therapeutic manipulation of peroxynitrite attenuates the development of opiate-induced antinociceptive tolerance, J. Clin. Invest 2007 117:1-11.
Modrak D E, Measurement of ceramide and sphingolipid metabolism in tumors: potential modulation of chemotherapy, Methods Mol Med 2005 111:183-94
Narita M, Yoshida T, Nakajima M, Narita M, Miyatake M, Takagi T, Yajima Y and Suzuki T, Direct evidence for spinal cord microglia in the development of a neuropathic pain-like state in mice. J Neurochem 2006 97:1337-1348.
Ndengele M M, Muscoli C, Wang Z Q, Doyle T M, Matuschak G M and Salvemini D, Superoxide potentiates NE-kappaB activation and modulates endotoxin-induced cytokine production in alveolar macrophages. Shock 2005 23:186-193.
Ossipov M H, Lai J, King T, Vanderah T W, Malan T P. Jr., Hruby V J, Porreca F. Antinociceptive and nociceptive actions of opioids. J Neurobiol 2004; 61: 126-148.
Ossipov M H, Lai J, Vanderah T W, Porreca F. Induction of pain facilitation by sustained opioid exposure: relationship to opioid antinociceptive tolerance. Life Sci 2003; 73:783-800.
Pahan K, Sheikh F G, Khan M, Namboodiri A M and Singh I, Sphingomyelinase and ceramide stimulate the expression of inducible nitric-oxide synthase in rat primary astrocytes. J Biol Chem 1998 273:2591-2600.
Pasternak G W, Nitric oxide and opioid tolerance, NIDA Res Monogr 1995 147:182-19.4.
Pautz A, Franzen R, Dorsch S, Boddinghaus B, Briner V A, Pfeilschifter J and Huwiler A, Cross-talk between nitric oxide and superoxide determines ceramide formation and apoptosis in glomerular cells. Kidney Int 2002 61:790-796.
Petrache I, Nataraian V, Zhen L, Medler R, Richter A T, Cho C, Hubbard W C, et al. Ceramide upregulation causes pulmonary cell apoptosis and emphysema-like disease in mice. Nat Med 2005, 11:491-498.
Radi R, Nitric oxide, oxidants, and protein tyrosine nitration. Proc Natl Acad Sci U S A 2004 101:4003-4008.
Renfrey S, Downton C and Featherstone J, The painful reality. Nat Rev Drug Discov 2003 2:175-176.
Rother J, van Echten G, Schwarzmann G and Sandhoff K, Biosynthesis of sphingolipids: dihydroceramide and not sphinganine is desaturated by cultured cells. Biochem Biophys Res Commun 1992 189:14-20.
Rothman R B. A review of the role of anti-opioid peptides in morphine tolerance and dependence. Synapse 1992; 12:129-138.
Salvemini D (2001) Analgesic methods using synthetic catalysts for the dismutation of superoxide radicals, in U.S. Pat. No. 6,180,620.
Salvemini D, Wang Z Q, Stern M K, Currie M G and Misko T P, Peroxynitrite decomposition catalysts: therapeutics for peroxynitrite-mediated pathology. Proc Natl Acad Sci U S A 1998 95:2659-2663.
Sarin P S, Agrawal S, Civeira M P, Goodchild J, Ikeuchi T and Zamecnik P C, Proc Natl Acad Sci U S A. 1988 85:7448-7451
Sarver N, Chang P S, Zaia J A, Ladne P A, Stephens D A and Rossi J J, Ribozymes as potential anti-HIV-1 therapeutic agents, Science 1990 247:1222-1225.
Song P and Zhao Z Q, The involvement of glial cells in the development of morphine tolerance. Neurosci Res 2001 39:281-286.
Schissel S L, Jiang X, Tweedie-Hardman J, Jeong T, Camejo E H, Najib J, Rapp J H, Secretory sphingomyelinase, a product of the acid sphingomyelinase gene, can hydrolyze atherogenic lipoproteins at neutral pH. Implications for atherosclerotic lesion development. J Biol Chem 1998; 273:2738-2746.
Schissel S L, Schuchnian E H, K J, Tabas I. Zn2+-stimulated sphingomyelinase is secreted by many cell types and is a product of the acid sphingomyelinase gene. J Biol Chem 1996; 271:18431-18436.
Schutze S, Potthoff K, Machleidt T, Berkovic D, Wiegrnann K, Kronke M. TNF activates NF-kappa B by phosphatidylcholine-specific phospholipase C-induced “acidic” sphingomyelin breakdown. Cell 1992; 71:765-776.
Simonnet G, Rivat C. Opioid-induced hyperalgesia: abnormal or normal pain? Neuroreport 2003; 14:1-7.
Smithies O, Gregg R G, Boggs S S, Koralewski M A and Kucherlapati R S, Insertion of DNA sequences into the human chromosomal beta-globin locus by homologous recombination, Nature 1985 317:230-234
Stein C A and Cohen J S, Oligodeoxynucleotides as inhibitors of gene expression: a review, Cancer Res. 1988 48:2659-2668
Tafesse F G, Temes P, Holthuis J C. The multigenic sphingomyelin synthase family. J Biol Chem 2006; 281:29421-29425.
Taylor D A, Fleming W W. Unifying perspectives of the mechanisms underlying the development of tolerance and physical dependence to opioids. J Pharmacol Exp Ther 2001; 297:11-18.
Thomas K R and Capecchi M R, Site-directed mutagenesis by gene targeting in mouse embryo-derived stern cells, Cell 1987 51:503-512.
Thompson S, Clarke A R, Pow A M, Hooper M L and Melton D W, Germ line transmission and expression of a corrected HPRT gene produced by gene targeting in embryonic stem cells, Cell 1989 56:313-321.
Vanderah T W, Ossipov M H, Lai Malan T P, Jr., Porreca F. Mechanisms of opioid-induced pain and antinociceptive tolerance: descending facilitation and spinal dynorphin. Pain 2001; 92:5-9.
van der Krol A R, Mol and Stuitje A R, Modulation of eukaryotic gene expression by complementary RNA or DNA sequences, Biotechniques 1988 6:958-976.
Vaughan T J, Williams A J, Pritchard K, Osbourn J K, Pope A R, Earnshaw J C, McCafferty J, Hodits R A, Wilton J, Johnson K S, Human Antibodies With Sub-nanomolar Affinities Isolated From a Large Non-immunized Phage Display Library, Nat Biotechnol 1996, 14:309-314.
Vera-Portocarrero L P, Zhang E T, King T, Ossipov M H, Vanderah T W, Lai J, Porreca F. Spinal NK-1 receptor expressing neurons mediate opioid-induced hyperalgesia and antinociceptive tolerance via activation of descending pathways, Pain 2006.
Wagner R W, Gene inhibition using antisense oligodeoxynucleotides, Nature. 1994 372:333-335
Wang Z Q, Porreca F, Cuzzocrea S, Galen K, Lightfoot R, Masini E, Muscoli C, Mollace V, Ndengele M, Ischiropoulos H and Salvemini D, A newly identified role for superoxide in inflammatory pain. J Pharmacol Exp Ther 2004 309:869-878
Watkins L R, Hutchinson M R, Ledeboer A, Wieseler-Frank J, Milligan E D and Maier S F, Norman Cousins Lecture. Glia as the “bad guys”: implications for improving clinical pain control and the clinical utility of opioids. Brain Behav Immun 2007 21:131-146.
Williams R D, Wang E and Merrill A H, Jr. Enzymology of long-chain base synthesis by liver: characterization of serine palmitoyltransferase in rat liver microsomes. Arch Biochem Biophys 1984 228:282-291.
Wright L and Kearney P, Current status of ribozymes as gene therapy agents for cancer Cancer Invest 2001, 19:495-509.
Yamakura F, Matsumoto T, Fujimura T, Taka H, Murayama K, Imai T and Uchida K, Modification of a single tryptophan residue in human Cu,Zn-superoxide dismutase peroxynitrite in the presence of bicarbonate. Biochim Biophys Acta 2001 1548:38-46.
Yamakura F, Taka H, Fujimura T and Murayama K, Inactivation of human manganese-superoxide dismutase by peroxynitrite is caused by exclusive nitration of tyrosine 34 to 3-nitrotyrosine. J Biol Chem 1998 273:14085-14089.
Zon G, Oligonucleotide analogues as potential chemotherapeutic agents, Pharm Res. 1988 5:539-549

What is claimed is:

1. A method for reducing, preventing or delaying the development of tolerance to, and/or physical dependence on an opioid drug upon administration of the opioid drug to a subject, the method comprising: administering to a subject in need thereof, an analgesic amount of the opioid drug and a therapeutically effective amount of an agent that inhibits ceramide biosynthesis.

2. A method according to claim 1, wherein the agent that inhibits ceramide biosynthesis is administered to the subject at a therapeutically effective time with respect to administering the opioid drug to the subject.

3. A method according to claim 1, wherein the ceramide synthesis inhibitor is administered from about 15 minutes to about 24 hours before administering the opioid drug.

4. A method according to claim 1, wherein the ceramide synthesis inhibitor is administered about 15 minutes before administering the opioid drug.

5. A method according to claim 1, wherein the ceramide synthesis inhibitor is administered about 2 hours before administering the opioid drug.

6. A method according to claim 1, wherein the ceramide synthesis inhibitor is administered about 24 hours before administering the opioid drug.

7. A method according to claim 1, wherein the ceramide synthesis inhibitor is administered at substantially the same time the opioid drug is administered.

8. A method according to claim 1, wherein the ceramide synthesis inhibitor is administered from about 15 minutes to about 24 hours after administering the opioid drug.

9. A method according to claim 1, wherein the ceramide synthesis inhibitor is administered about 15 minutes after administering the opioid drug.

10. A method according to claim 1, wherein the ceramide synthesis inhibitor is administered about 2 hours after administering the opioid drug.

11. A method according to claim 1, wherein the ceramide synthesis inhibitor is administered about 24 hours after administering the opioid drug.

12. A method according to claim 1, wherein the opioid drug comprises one or more opioid drugs selected from the group consisting of alfentanil, allylprodine, alphaprodine, anileridine, benzylmorphine, bezitramide, buprenorphine, butorphanol, clonitazene, codeine, cyclazocine, desomorphine, dextromoramide, dezocine, diampromide, diamorphone, dihydrocodeine, dihydromorphine, dimenoxadol, dimepheptanol, dimethylthiambutene, dioxaphetylbutyrate, dipipanone, eptazocine, ethoheptazine, ethylmethylthiambutene, ethylmorphine, etonitazene, fentanyl, heroin, hydrocodone, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papaveretum, pentazocine, phenadoxone, phenomorphan, phenazocine, phenoperidine, piminodine, piritramide, propheptazine, promedol, properidine, propiram, propoxyphene, sufentanil, tilidine, and tramadol.

13. A method according to claim 1, wherein the agent that inhibits ceramide biosynthesis is an antisense nucleic acid, a ribozyme, a triplex-forming oligonucleotide, a siRNA, a probe, a primer, an antibody or a combination thereof.

14. A method according to claim 1, wherein the agent that inhibits ceramide biosynthesis is a serine palmitoyltransferase inhibitor selected from the group consisting of sphingo-fungins, lipoxamycin, myriocin, L-cycloserine, beta-chloro-L-alanine, Viridiofungins, and combinations thereof.

15. A method according to claim 1, wherein the agent that inhibits ceramide biosynthesis is a dihydroceramide desaturase inhibitor selected from the group consisting of GT11, GT85, GT98, GT99, GT55, GT77, and mixtures thereof.

16. A method according to claim 1, wherein the agent that inhibits ceramide biosynthesis is a sphingomyelinase inhibitor selected from the group consisting of L-alpha-phosphatidyl-D-myoinositol-3,5-bisphosphate, L-alpha-phosphatidyl-D-myo-inositol-3,4,5-triphosphate, ceramide1-phosphate, sphingosine-1-phosphate, glutathione, desipramine, imipramine, SR33557, (3-carbazol-9-yl-propyl)-[2-(3,4dimethoxy-phenyl)-ethyl)-methyl-amine, hexanoic acid (2-cyclo-pent-1-enyl-2-hydroxy-1-hydroxy-methylethyl)-amide, GW4869, scyphostatin, macquarimicin A, alutenusin, chlorogentisylquinone, manumycin A, a-Mangostin, sphingotactones, 3-O-methylsphingomyelin, 3-O-ethylsphingomyelin, [3(10,11-Dihydro-dibenzo[b,f]azepin-5-yl)-N-propyl]-[2(3,4-dimethoxyphenyl)ethylmethylamine, [3(10,11-Dihydro-dibenzo[b,f]azepin-5-yl)-N-propyl]-[2(4-methoxyphenyl)ethyl]methylamine, [2(3,4-Dimethoxyphenyl)-ethyl]-[3(2-chlorphenothiazin-10-yl)-N-propyl]-methylamine, [2(4-Methoxyphenyl)-ethyl]-[3(2-chlorphenothiazin-10-yl)-N-propyl]-methylamine, [3(Carbazol-9-yl)-N-propyl]-[2(3,4-dimethoxyphenyl)-ethyl]methylamine, [3 (Carbazol-9-yl)-N-propyl]-[2(4-methoxyphenyl)-ethyl]methylamine, [2(3,4-Dimethoxyphenyl)-ethyl]-[2(phenothiazin-10-yl)-N-ethyl]-methylamine, [2 (4-Methoxyphenyl)-ethyl]-[2 (phenothiazin-10-yl)-N-ethyl]-methylamine, [(3,4-Dimethoxyphenyl)-acetyl]-[3(2-chlorphenothiazin-10-yl)-N-propyl]-methylamine, n-(1-naphthyl)-N′[2(3,4-dimethoxyphenyl)-ethyl]-ethyl diamine, n-(1-naphthyl)-N[20-methoxyphenyl)-ethyl]-ethyl diamine, n-[2(3,4-Dimethoxyphenyl)-ethyl]-n-[1-naphthylmethyl]amine, n-[2(4-Methoxyphenyl)-ethyl]-n-[1 amine, [3(10,11-Dihydro dibenzo[b,f]azepin-5-yl)-N-propyl]-[(4-methoxyphenyl)-acetyl]methylamine, [2(10,11-Dihydro-dibenzo[b,f]azepin-5-yl)-N-ethyl]-[2(3,4-dimethoxyphenyl)ethyl]methylamine, [2(10,11-Dihydro-dibenzo[b,f]azepin-5-yl)-N-ethyl]-[2(4-methoxyphenyl)-ethyl]methylamine, [2(10,11-Dihydro-dibenzo[b,f]azepin-5-yl)-N-ethyl]-[(4-methoxyphenyl)-acety-1]methylamine, n-[2(Carbazol-9-yl)-N-ethyl]-N′[2(4-methoxyphenyl)-ethyl]piperazine, 1 [2(Carbazol-9-yl)-N-ethyl]-4[2(4-methoxyphenyl)-ethyl]-3,5-dimethylpiperazine, [2(4-Methoxyphenyl)-ethyl]-[3(phenoxazin-10-yl)-N-propyl]-methylamine, [3(5,6,11,12-Tetrahydrodibenzo[b,f]azocin)-N-propyl]-[3(4-methoxyphenyl)propyl]methylamine, n-(5H-Dibenzo[A,D]cycloheptan-5-yl)-N′[2(4-methoxyphenyl)-ethyl]-propylene diamine, [2(Carbazol-9-yl)-N-ethyl]-[2(4-methoxyphenyl)-ethyl]methylamine, and combinations thereof.

17. A method for reducing, preventing or delaying the development of tolerance to, and/or physical dependence on morphine upon administration of the morphine to a subject, the method comprising: administering to a subject in need thereof, an analgesic amount of the morphine and a therapeutically effective amount of an agent comprising a compound selected from the group consisting of FB1, D609, myriocin and combinations thereof.

18. A method according to claim 18, wherein the agent comprising a compound selected from the group consisting of FB1, D609, myriocin and combinations thereof is administered to the subject at a therapeutically effective time with respect to administering the opioid drug to the subject.

19. A method for reducing, preventing or delaying the development of tolerance to, and/or physical dependence on an opioid drug upon administration of the opioid drug to a subject, the method comprising: administering to a subject in need thereof an analgesic amount of the opioid drug and a therapeutically effective amount of an agent that inhibits ceramide biosynthesis, wherein the agent that inhibits ceramide biosynthesis targets at least one ceramide-biosynthetic enzyme selected from the group consisting of a sphingomyelinase, serine palmitoyltransferase, 3-ketosphinganine reductase, ceramide synthase, dihydroceramide desaturase, and combinations thereof.

20. A method according to claim 19, wherein the agent that inhibits ceramide biosynthesis is administered to the subject at a therapeutically effective time with respect to administering the opioid drug to the subject.


SEQUENCE LISTING (36 sequences)

SEQ ID NO: 1 - protein, 631 aa, Homo Sapiens
MPRYGASLRQSCPRSGREQGQDGTAGAPGLLWMGLVLALALALALALALSDSRVLWAPAEAHPLSPQGHPARLHRIVPRLRDVFGWGNLTCPICKGLFTAINLGLKKEPNVARVGSVAIKLCNLLKIAPPAVCQSIVHLFEDDMVEVWRRSVLSPSEACGLLLGSTCGHWDIFSSWNISLPTVPKPPPKPPSPPAPGAPVSRILFLTDLHWDHDYLEGTDPDCADPLCCRRGSGLPPASRPGAGYWGEYSKCDLPLRTLESLLSGLGPAGPFDMVYWTGDIPAHDVWHQTRQDQLRALTTVTALVRKFLGPVPVYPAVGNHESTPVNSFPPPFIEGNHSSRWLYEAMAKAWEPWLPAEALRTLRIGGFYALSPYPGLRLISLNMNFCSRENFWLLINSTDPAGQLQWLVGELQAAEDRGDKVHIIGHIPPGHCLKSWSWNYYRIVARYENTLAAQFFGHTHVDEFEVFYDEETLSRPLAVAFLAPSATTYIGLNPGYRVYQIDGNYSGSSHVVLDHETYILNLTQANIPGAIPHWQLLYRARETYGLPNTLPTAWHNLVYRMRGDMQLFQTFWFLYHKGHPPSEPCGTPCRLATLCAQLSARADSPALCRHLMPDGSLPEAQSLWPRPLFC

SEQ ID NO: 2 - DNA, 2473 bp, Homo sapiens
atcagaggaagaggaaggggcggagctgctttgcggccggccgcggagcagtcagccgactacagagaagggtaatcgggtgtccccggcgccgcccggggccctgagggctggctagggtccaggccgggggggacgggacagacgaaccagccccgtgtaggaagcgcgacaatgccccgctacggagcgtcactccgccagagctgccccaggtccggccgggagcagggacaagacgggaccgccggagcccccggactcctttggatgggcctggtgctggcgctggcgctggcgctggcgctggcgctggctctgtctgactctcgggttctctgggctccggcagaggctcaccctctttctccccaaggccatcctgccaggttacatcgcatagtgccccggctccgagatgtctttgggtgggggaacctcacctgcccaatctgcaaaggtctattcaccgccatcaacctcgggctgaagaaggaacccaatgtggctcgcgtgggctccgtggccatcaagctgtgcaatctgctgaagatagcaccacctgccgtgtgccaatccattgtccacctctttgaggatgacatggtggaggtgtggagacgctcagtgctgagcccatctgaggcctgtggcctgctcctgggctccacctgtgggcactgggacattttctcatcttggaacatctctttgcctactgtgccgaagccgccccccaaaccccctagccccccagccccaggtgcccctgtcagccgcatcctcttcctcactgacctgcactgggatcatgactacctggagggcacggaccctgactgtgcagacccactgtgctgccgccggggttctggcctgccgcccgcatcccggccaggtgccggatactggggcgaatacagcaagtgtgacctgcccctgaggaccctggagagcctgttgagtgggctgggcccagccggcccttttgatatggtgtactggacaggagacatccccgcacatgatgtctggcaccagactcgtcaggaccaactgcgggccctgaccaccgtcacagcacttgtgaggaagttcctggggccagtgccagtgtaccctgctgtgggtaaccatgaaagcacacctgtcaatagcttccctccccccttcattgagggcaaccactcctcccgctggctctatgaagcgatggccaaggcttgggagccctggctgcctgccgaagccctgcgcaccctcagaattggggggttctatgctctttccccataccccggtctccgcctcatctctctcaatatgaatttttgttcccgtgagaacttctggctcttgatcaactccacggatcccgcaggacagctccagtggctggtgggggagcttcaggctgctgaggatcgaggagacaaagtgcatataattggccacattcccccagggcactgtctgaagagctggagctggaattattaccgaattgtagccaggtatgagaacaccctggctgctcagttctttggccacactcatgtggatgaatttgaggtcttctatgatgaagagactctgagccggccgctggctgtagccttcctggcacccagtgcaactacctacatcggccttaatcctggttaccgtgtgtaccaaatagatggaaactactccgggagctctcacgtggtcctggaccatgagacctacatcctgaatctgacccaggcaaacataccgggagccataccgcactggcagcttctctacagggctcgagaaacctatgggctgcccaacacactgcctaccgcctggcacaacctggtatatcgcatgcggggcgacatgcaacttttccagaccttctggtttctctaccataagggccacccaccctcggagccctgtggcacgccctgccgtctggctactctttgtgcccagctctctgcccgtgctgacagccctgctctgtgccgccacctgatgccagatgggagcctcccagaggcccagagcctgtggccaaggccactgttttgctagggccccagggcccacatttgggaaagttcttgatgtaggaaagggtgaaaaagcccaaatgctgctgtggttcaaccaggcaagatcatccggtgaaagaaccagtccctgggccccaaggatgccggggaaacaggaccttctcctttcctggagctggtttagctggatatgggagggggtttggctgcctgtgcccaggagctagactgccttgaggctgctgtcctttcacagccatggagtagaggcctaagttgacactgccctgggcagacaagacaggagctgtcgccccaggcctgtgctgcccagccaggaaccctgtactgctgctgcgacctgatgctgccagtctgttaaaataaagataagagacttggactccaaaaaaaaaaaaaaa

SEQ ID NO: 3 - protein, 423 aa, Homo sapiens
MKLNFSLRLRIFNLNCWGIPYLSKHRADRMRRLGDFLNQESFDLALLEEVWSEQDFQYLRQKLSPTYPAAHHFRSGIIGSGLCVFSKHPIQELTQHIYTLNGYPYMIHHGDWFSGKAVGLLVLHLSGMVLNAYVTHLHAEYNRQKDIYLAHRVAQAWELAQFIHHTSKKADVVLLCGDLNMHPEDLGCCLLKEWTGLHDAYLETRDFKGSEEGNTMVPKNCYVSQQELKPFPFGVRIDYVLYKAVSGFYISCKSFETTTGFDPHSGTPLSDHEALMATLFVRHSPPQQNPSSTHGPAERSPLMCVLKEAWTELGLGMAQARWWATFASYVIGLGLLLLALLCVLAAGGGAGEAAILLWTPSVGLVLWAGAFYLFHVQEVNGLYRAQAELQHVLGRAREAQDLGPEPQPALLLGQQEGDRTKEQ

SEQ ID NO: 4 - DNA, 1662 bp, Homo sapiens
gcggccgcgaccgccggggacgagcttggaggaaaaggaaccgggagccgcccacccgggggcgctctccggacccccagggtcctagcgcgcggcccttaccgagcctgggcgcccggatttcggsagcggatcgcctttccgggttggcggcccgcctgattgggaacagccggccggttgccgggggaacgcgggagtcgggcccgacctgagccacgcgggcttggtgcccacctgtgcgcgccgcctgcgaagaaggaacggtctagggagaaggcgccgccggccgcccccgtccccaccgcggccgtcgctggagagttcgagccgcctagcgcccctggagctccccaaccatgaagctcaacttctccctgcgactgcggatcttcaacctcaactgctggggcattccgtacttgagcaagcaccgggccgaccgcatgaggcgcctgggagactttctgaaccaggagagcttcgacctggctttgctggaggaggtgtggagtgagcaggacttccagtacctgagacagaagctgtcacctacctacccagctgcacaccacttccggagcggaatcattggcagtggcctctgtgtcttctccaaacatccaatccaggagcttacccagcacatctacactctcaatggctacccctacatgatccatcatggtgactggttcagtgggaaggctgtggggctgctggtgctccatctaagtggcatggtgctcaacgcctatgtgacccatctccatgccgaatacaatcgacagaaggacatctacctagcacatcgtgtggcccaagcttgggaattggcccagttcatccaccacacatccaagaaggcagacgtggttctgttgtgtggagacctcaacatgcacccagaagacctgggctgctgcctgctgaaggagtggacagggcttcatgatgcctatcttgaaactcgggacttcaagggctctgaggaaggcaacacaatggtacccaagaactgctacgtcagccagcaggagctgaagccatttccctttggtgtccgcattgactacgtgctttacaaggcagtttctgggttttacatctcctgtaagagttttgaaaccactacaggctttgaccctcacagtggcacccccctctctgatcatgaagccctgatggctactctgtttgtgaggcacagccccccacagcagaaccccagctctacccacggaccagcagagaggtcgccgttgatgtgtgtgctaaaggaggcctggacggagctgggtctgggcatggctcaggctcgctggtgggccaccttcgctagctatgtgattggcctggggctgcttctcctggcactgctgtgtgtcctggcggctggaggaggggccggggaagctgccatactgctctggacccccagtgtagggctggtgctgtgggcaggtgcattctacctcttccacgtacaggaggtcaatggcttatatagggcccaggctgagctccagcatgtgctaggaagggcaagggaggcccaggatctgggcccagagcctcagccagccctactcctggggcagcaggagggggacagaactaaagaacaataaagcttggccctttaaaaaaaaaaaaaaaaaa

SEQ ID NO: 5 - protein, 655 aa, Homo sapiens
MVLYTTPFPNSCLSALHCVSWALIFPCYWLVDRLAASFIPTTYEKRQRADDPCCLQLLCTALFTPIYLALLVASLPFAFLGFLFWSPLQSARRPYIYSRLEDKGLAGGAALLSEWKGTGPGKSFCFATANVCLLPDSLARVNNLFNTQARAKEIGQRIRNGAARPQIKIYIDSPTNTSISAASFSSLVSPQGGDGVARAVPGSIKRTASVEYKGDGGRHPGDEAANGPASGDPVDSSSPEDACIVRIGGEEGGRPPEADDPVPGGQARNGAGGGPRGQTPNHNQQDGDSGSLGSPSASRESLVKGRAGPDTSASGEPGANSKLLYKASVVKKAAARRRRHPDEAFDHEVSAFFPANLDFLCLQEVFDKRAATKLKEQLHGYFEYILYDVGVYGCQGCCSFKCLNSGLLFASRYPIMDVAYHCYPNKCNDDALASKGALFLKVQVGSTPQDQRIVGYIACTHLHAPQEDSAIRCGQLDLLQDWLADFRKSTSSSSAANPEELVAFDVVCGDFNFDNCSSDDKLEQQHSLFTHYRDPCRLGPGEEKPWAIGTLLDTNGLYDEDVCTPDNLQKVLESEEGRREYLAFPTSKSSGQKGRKELLKGNGRRIDYMLHAEEGLCPDWKAEVEEFSFITQLSGLTDHLPVAMRLMVSSGEEEA

SEQ ID NO: 6 - DNA, 5269 bp, Homo sapiens
gctgagtctgagggaggctccggacccgagagccgcgagagccgccgccgctgcggccgccgccagatctgcggccgggagcccgggctgtgaggagccgggaggagcggggtgcgctgccgggcgctgaccgccctcccgcccgccgtcagaggtctgcggtgacagctcttcttcagagagaaggacaacaaggtcccagtggcccctcctcagggtctgcagtaggcctccgcatggcccaccgaggtgaaccatgaccggctggccaacattcgccattgaccagccggagttgcatctcgccaggaggtgacccctcctccagctgcccccaactcgcccaccctcgcccaggaaagtgcccgcagctgccacggacaccatgtagtagggccggctgcggcgcccagtgagctgcgatggttttgtacacgaccccctttcctaacagctgtctgtccgccctgcactgtgtgtcctgggcccttatctttccatgctactggctggtggaccggctcgctgcctccttcatacccaccacctacgagaagcgccagcgggcagacgacccgtgctgcctgcagctgctctgcactgccctcttcacgcccatctacctggccctcctggtggcctcgctgccctttgcgtttctcggctttctcttctggtccccactgcagtcggcccgccggccctacatctattcacggctggaagacaagggcctggccggtggggcagccctgctcagtgaatggaagggcacggggcctggcaaaagcttctgctttgccactgccaacgtctgcctcctgcccgactcactcgccagggtcaacaacctttttaacacccaagcgcgggccaaggagatcgggcagagaatccgcaatggggccgcccggccccagatcaaaatttacatcgactcccccaccaatacctccatcagcgccgctagcttcagcagcctggtgtcaccacagggcggcgatggggtggcccgggccgtccccgggagcattaagaggacagcctctgtggagtacaagggtgacggtgggcggcaccccggtgacgaggctgccaacggcccagcctctggggaccctgtcgacagcagcagcccggaggatgcctgcatcgtgcgcatcggtggcgaggagggcggccggccacctgaagctgacgaccctgtgcctgggggccaggccaggaacggagctggcgggggcccaaggggccagacgcccaaccataatcagcaggacggggattcagggagcctgggcagcccctcggcctcccgggagtccctggtgaaggggcgagctgggccagacaccagtgccagcggggagccaggtgccaacagcaagctcctgtacaaggcctcggtggtgaagaaggcggctgcacgcaggaggcggcaccccgacgaggccttcgaccatgaggtctccgccttcttccccgccaacctggacttcctgtgcctgcaggaggtgtttgacaagcgagcagccaccaaattgaaagagcagctgcacggctacttcgagtacatcctgtacgacgtcggggtctacggctgccagggctgctgcagcttcaagtgtctcaacagcggcctcctctttgccagccgctaccccatcatggacgtggcctatcactgttaccccaacaagtgtaacgacgatgccctggcctctaagggagctctgtttctcaaggtgcaggtgggaagcacacctcaggaccaaagaatcgtcgggtacatcgcctgcacacacctgcatgccccgcaagaggacagcgccatccggtgtgggcagctggacctgcttcaggactggctggctgatttccgaaaatctacctcctcgtccagcgcagccaaccccgaggagctggtggcatttgacgtcgtctgtggagatttcaactttgataactgctcctctgacgacaagctggagcagcaacactccctgttcacccactacagggacccctgccgcctggggcctggtgaggagaagccgtgggccatcggtactctgctggacacgaacggcctgtacgatgaggatgtgtgcacccccgacaacctgcagaaggtcctggagagtgaggagggccgcagggagtacctggcgtttcccaccagcaagagctcgggccagaaggggcggaaggagctgctgaagggcaacggccggcgcatcgactacatgctgcatgcagaggaggggctgtgcccagactggaaggccgaggtggaagaattcagttttatcacccagctgtccggcctgacggaccacctgccagtagccatgcgactgatggtgtcttcgggggaggaggaggcatagaccgtccggagcagcggggcctctgccagcccttgcagctgcagcccatccctgggccatgtcccctccatcgagtgcccggtgcttgggggaggagggcagggacagggagggagccacagtcagtgcccgggaacctggaagctgcgctgctctgcgcctctgggcctcactgtggacagaggagtcaggcccgccccaggagcctccagctgcctaaccagtgccattctttcacaacacgattttctacaaatctacagcacaaccgagtttgtaacccgtgggttagtatgaggaccgggttcgtgtactctctgtatctcctcttaagcttcgtccagggttctttatttttgtctgctgccaatgtcgtctcgcatgcctgcaccctcgcatgcacgctgcccgcatgccacgtgccacgctgtagccacagaccccttgctcgggcctcacccaaggccaaactccaaacacaatcagaaccagccaaagaagcacttcctgggcacggccaccagctctcccgcctccagtgtgggccggctcctgcagggtccgagggctgcatctctaccagccagcccagggctcttcccagggtctcgcattcaagggcaattacattttaaaaagaaaaacagaaaaaggttaatcacaaaaccaaccctcacttcacagggtctgtaagtcactcatagaactttgctcttcccgagacagggtcccttccccagctcaggcacaacagagtctggcaggctctggcaccctgggcctcctccgggagcctcccatctgggcagtggagccataaacggggatccgagaagagagtatccacttttttttttacaggaagaagggactcacagcataaacgggggtgggggggatcctgattttgaaaataatctatttgtagcttctcttctatcaaaaccaacacatcctcttctttctgccaatcctctcccccacgggacacctctctggttcgggaccaatccctccctggggacgtgccccacctgcgtgccggctgagctcaggaacccctgcctgccccccgggtggggctgcggctctggcctcccaggcccatcctcaacagctaccccagccaacaccaaggccacaaggggaccccggcctaggaggcaggaagccaaggtgcagagagcagcctggccctcaccagtgcgcaagctggggcagcaaggctgacagttgctgcatgcccagggcagggtgtggtactggcacccaagttcagcatggcagagctggccaacagcttgtggtccccgatctgcctccagccccaagatgcctacagcccccaggccccttcggcagcactgcctctgcccacctgcctttaagagactccagggctgctcctgtcatgcagcgaaggttttgtctgtttcaaagttcgagactcaacttgagggactgtttttgacaatccccgctgacctccgctcctcgtggcgccctggccctacacccagcctggcccagggccggctttgcctggtgaggctggagggagcaccaggacctgctgtctgctgtcagcccctcctggtgctggtgccctgatgctgtgccttgtcacccattgagctgcaagagggaccaagagggggccacgcagccagccagatgcctggccctgtgctggggcagacaacgctgcagagcccagggagcctggcgctaggacgtgcgtccttgtgacactggcctgtctgaactcacctggcctgggaagcaccgtctgcccgggcccaagccctgcccctccagagtccagagccaggaaggggctgctgagggcgagcatcctgctgggctctctgcccggcccacccctccaaggggctggcctgtgagccttgactgggattcatgatgtggaggcccccaacttccagaagcagctggtactctgctcacacaagcgactgggccggccggccctggacccctagaccccgagccgcctgccgactgcctgcacagggagagcagttgaggcccgggcagggcccccacaccagaccccaacatagcttccccacccaggcaccccctcccggggcagcaggcgtgggagtcagggctgcatgctcctcccctcccacctcacaggcggccttaggcaagtcattttctgtcatcacaaggtcgcctctgcctagtcaggtcctggggtccagagtaaggatgtgcggcccccaggcccccgcacacctccctcagcaccaagaccgggacccccccacccacgtgtctcattgtggctgcctatggactcccgggccttgtgtgcaggccaggcccttccactgattttttaaagtgaaccattgctggatctcagattctgtggcatctaaggcctagcaggggtgggcacacgggtcacccgaggcccataccaagactctgttcctgccctaggcccagtctcaaaggaagccacaaggcgcgggggccactgaggaaggaaatgttcattttcatttgtccaaaaccaccttaagttttaagtatattaatcttgatgctttttaactattgctttttaacttgctgagatttagaaatactgttataaaaacttttttaatttctgtatttttttctgtattgtatcttcatgggacattaggggttttctatggtaagcacacctatggttttggtaaaaacattatcaaatatatatccagacggttcttccctagaagaaaaacaagtctttacacctgataaaatattttgcgaagagaggtgttctttttccttactggtgctgaaaggaaggatggataacgaggagaaaataaaactgtgaggctcaa

SEQ ID NO: 7 - protein, 453 aa, Homo sapiens
MALVRALVCCLLTAWHCRSGLGLPVAPAGGRNPPPAIGQFWHVTDLHLDPTYHITDDHTKVCASSKGANASNPGPFGDVLCDSPYQLILSAFDFIKNSGQEASFMIWTGDSPPHVPVPELSTDTVINVITNMTTTIQSLFPNLQVFPALGNHDYWPQDQLPVVTSKVYNAVANLWKPWLDEEAISTLRKGGFYSQKVTTNPNLRIISLNTNLYYGPNIMTLNKTDPANQFEWLESTLNNSQQNKEKVYIIAHVPVGYLPSSQNITAMREYYNEKLIDIFQKYSDVIAGQFYGHTHRDSIMVLSDKKGSPVNSLFVAPAVTPVKSVLEKQTNNPGIRLFQYDPRDYKLLDMLQYYLNLTEANLKGESIWKLEYILTQTYDIEDLQPESLYGLAKQFTILDSKQFIKYYNYFFVSYDSSVTCDKTCKAFQICAIMNLDNISYADCLKQLYIKHNY

SEQ ID NO: 8 - DNA, 2210 bp, Homo sapiens
gctttgcaaccagctgcggatgcctggacgtctaattgctcaaaggtgttatctgagactgaagaaacagacttttcccctggattctttgaaaatccccctctcttattctctagcagaataaaaactccctgctttttcctttttacacacggatttgagagatcttgttctccctccttctggcttttactaaattgaatgtctttctctatccccaagcaccagtatgtcagtgtttgacatcaactgcaccactgatacacgagtcggaatttgagcttctacaagtacattccttcctaggccaaacactgacgctaagaaatacgagaacagatcatcgctaaacagcagctgaaggtcaggcgaactgactcgctgcggaatctgcctttgcacgtgatcagtcggacgtctacacccgcagccgtcttctgtctccgcctcaccctcaggcctgacggtccgagtggagctgcgggacagcccgaacctccaggtcagccccgcggccctccatggcgctggtgcgcgcactcgtctgctgcctgctgactgcctggcactgccgctccggcctcgggctgcccgtggcgcccgcaggcggcaggaatcctcctccggcgataggacagttttggcatgtgactgacttacacttagaccctacttaccacatcacagatgaccacacaaaagtgtgtgcttcatctaaaggtgcaaatgcctccaaccctggcccttttggagatgttctgtgtgattctccatatcaacttattttgtcagcatttgattttattaaaaattctggacaagaagcatctttcatgatatggacaggggatagcccacctcatgttcctgtacctgaactctcaacagacactgttataaatgtgatcactaatatgacaaccaccatccagagtctctttccaaatctccaggttttccctgcgctgggtaatcatgactattggccacaggatcaactgcctgtagtcaccagtaaagtgtacaatgcagtagcaaacctctggaaaccatggctagatgaagaagctattagtactttaaggaaaggtggtttttattcacagaaagttacaactaatccaaaccttaggatcatcagtctaaacacaaacttgtactacggcccaaatataatgacactgaacaagactgacccagccaaccagtttgaatggctagaaagtacattgaacaactctcagcagaataaggagaaggtgtatatcatagcacatgttccagtggggtatctgccatcttcacagaacatcacagcaatgagagaatactataatgagaaattgatagatatttttcaaaaatacagtgatgtcattgcaggacaattttatggacacactcacagagacagcattatggttctttcagataaaaaaggaagtccagtaaattctttgtttgtggctcctgctgttacaccagtgaagagtgttttagaaaaacagaccaacaatcctggtatcagactgtttcagtatgatcctcgtgattataaattattggatatgttgcagtattacttgaatctgacagaggcgaatctaaagggagagtccatctggaagctggagtatatcctgacccagacctacgacattgaagatttgcagccggaaagtttatatggattagctaaacaatttacaatcctagacagtaagcagtttataaaatactacaattacttctttgtgagttatgacagcagtgtaacatgtgataagacatgtaaggcctttcagatttgtgcaattatgaatcttgataatatttcctatgcagattgcctcaaacagctttatataaagcacaattactagtatttcacagtttttgctaatagaaaatgctgattctgattctgagatcaatttgtgggaattttacataaatctttgttaattactgagtgggcaagtagacttcctgtctttgctttctttttttttttctttttgatgccttaatgtagatatctttatcattctgaattgtattatatatttaaagtgctcattaatagaatgatggatgtaaattggatgtaaatattcagtttatataattatatctaatttgtacccttgttgaaattgtcatttatacaataaagcgaattctttatctctaaaaaaaaaaaaaaaaaa

SEQ ID NO: 9 - protein, 450 aa, Homo sapiens
MRLLAWLIFLANWGGARAEPGKFWHIADLHLDPDYKVSKDPFQVCPSAGSQPVPDAGPWGDYLCDSPWALINSSIYAMKEIEPEPDFILWTGDDTPHVPDEKLGEAAVLEIVERLTKLIREVFPDTKVYAALGNHDFHPKNQFPAGSNNIYNQIAELWKPWLSNESIALFKKGAFYCEKLPGPSGAGRIVVLNTNLYYTSNALTADMADPGQQFQWLEDVLTDASKAGDMVYIVGHVPPGFFEKTQNKAWFREGFNEKYLKVVRKHHRVIAGQFFGHHHTDSFRMLYDDAGVPISAMFITPGVTPWKTTLPGVVNGANNPAIRVFEYDRATLSLKDMVTYFMNLSQANAQGTPRWELEYQLTEAYGVPDASAHSMHTVLDRIAGDQSTLQRYYVYNSVSYSAGVCDEACSMQHVCAMRQVDIDAYTTCLYASGTTPVPQLPLLLMALLGL

SEQ ID NO: 10 - DNA, 1890 bp, Homo sapiens
ccagatcataccctgctgggcaaaggaggaagagccagaggatccagacgccttggaggacttggaacacctgtaacaggacaaggagttctgctcaggcacgtggccacagaaaactacttaggaagcctgtggtgagaacaacaacagtgcctgagaatcccacggctctggggaagtgagccccgaggatgaggctgctcgcctggctgattttcctggctaactggggaggtgccagggctgaaccagggaagttctggcacatcgctgacctgcaccttgaccctgactacaaggtatccaaagaccccttccaggtgtgcccatcagctggatcccagccagtgcccgacgcaggcccctggggtgactacctctgtgattctccctgggccctcatcaactcctccatctatgccatgaaggagattgagccagagccagacttcattctctggactggtgatgacacgcctcatgtgcccgatgagaaactgggagaggcagctgtactggaaattgtggaacgcctgaccaagctcatcagagaggtctttccagatactaaagtctatgctgctttgggaaatcatgattttcaccccaaaaaccagttcccagctggaagtaacaacatctacaatcagatagcagaactatggaaaccctggcttagtaatgagtccatcgctctcttcaaaaaaggtgccttctactgtgagaagctgccgggtcccagcggggctgggcgaattgtggtcctcaacaccaatctgtactataccagcaatgcgctgacagcagacatggcggaccctggccagcagttccagtggctggaagatgtgctgaccgatgcatccaaagctggggacatggtgtacattgtcggccacgtgcccccggggttctttgagaagacgcaaaacaaggcatggttccgggagggcttcaatgaaaaatacctgaaggtggtccggaagcatcatcgcgtcatagcagggcagttcttcgggcaccaccacaccgacagctttcggatgctctatgatgatgcaggtgtccccataagcgccatgttcatcacacctggagtcaccccatggaaaaccacattacctggagtggtcaatggggccaacaatccagccatccgggtgttcgaatatgaccgagccacactgagcctgaaggacatggtgacctacttcatgaacctgagccaggcgaatgctcaggggacgccgcgctgggagctcgagtaccagctgaccgaggcctatggggtgccggacgccagcgcccactccatgcacacagtgctggaccgcatcgctggcgaccagagcacactgcagcgctactacgtctataactcagtcagctactctgctggggtctgcgacgaggcctgcagcatgcagcacgtgtgtgccatgcgccaggtggacattgacgcttacaccacctgtctgtatgcctctggcaccacgcccgtgccccagctcccgctgctgctgatggccctgctgggcctgtgcacgctcgtgctgtgacctgccaggctcaccttcttcctggtaacgggtaacgggggcagcgcccaggatcacccagagctgggccttccaccatttcctccgcgcctgaggagtgaactgaaataggacaaccgaatcaggaagcgaagccccaggagctgcagccatccgtgatcgcgccactgcactccagcctgggcgacaaagccagactctctccaaaaacaaaccagaaacagaaaagaaatgacgacccaagacccccctacaagcatacttcttttgcgtattatgttttactcacaaaacaaagctcatcatgcgtttgaaaaaaaaaaaaaaaaaa

SEQ ID NO: 11 - protein, 837 aa, Homo sapiens
MTTFGAVAEWRLPSLRRATLWIPQWFAKKAIFNSPLEAAMAFPHLQQPSFLLASLKADSINKPFAQQCQDLVKVIEDFPAKELHTIFPWLVESIFGSLDGVLVGWNLRCLQGRVNPVEYSIVMEFLDPGGPMMKLVYKLQAEDYKFDFPVSYLPGPVKASIQECILPDSPLYHNKVQFTPTGGLGLNLALNPFEYYIFFFALSLITQKPLPVSLHVRTSDCAYFILVDRYLSWFLPTEGSVPPPLSSSPGGTSPSPPPRTPAIPFASYGLHHTSLLKRHISHQTSVNADPASHEIWRSETLLQVFVEMWLHHYSLEMYQKMQSPHAKESFTPTEEHVLVVRLLLKHLHAFANSLKPEQASPSAHSHATSPLEEFKRAAVPRFVQQKLYLFLQHCFGHWPLDASFRAVLEMWLSYLQPWRYAPDKQAPGSDSQPRCVSEKWAPFVQENLLMYTKLFVGFLNRALRTDLVSPKHALMVFRVAKVFAQPNLAEMIQKGEQLFLEPELVIPHRQHRLFTAPTFTGSFLSPWPPAVTDASFKVKSHVYSLEGQDCKYTPMFGPEARTLVLRLAQLITQAKHTAKSISDQCAESPAGHSFLSWLGFSSMDTNGSYTANDLDEMGQDSVRKTDEYLEKALEYLRQIFRLSEAQLRQFTLALGTTQDENGKKQLPDCIVGEDGLILTPLGRYQIINGLRRFEIEYQGDPELQPIRSYEIASLVRTLFRLSSAINHRFAGQMAALCSRDDFLGSFCRYHLTEPGLASRHLLSPVGRRQVAGHTRGPRLSLRFLGSYRTLVSLLLAFFVASLFCVGPLPCTLLLTLGYVLYASAMTLLTERGKLHQP

SEQ ID NO: 12 - DNA, 4182 bp, Homo sapiens
aacagttattgggcgctcacggtgtgctgagcggcgctctaggagccgggagtggcatggcggaccgcaggggtccgctgcttggcgatctgggcctccctgatagactgcattggtcgtgctcgcttaggtggcagcacccagcccagtgccaggcatacattgggcgctcggtaaatgcctctttcaagaaagcgtgcaattttctggtccgcaccggtgcaccactaggggtcgcttttcggggcgggcggggggaaggggggggcactaatcaacaatactgcttacgcgcacgcggattccttgctggggagaaagtaccttggggcgccggaggccgccacaacgcaggcgcattcagctaaggaccactccctcccccgcactcctgcctcgccatttctcttccccgcccggccggccttcgctttgcgcacgcgccttttgaggtaacggcccaaagaggtggaagcgcttttcccgcccggccgcggggcgtggctctgcgcgcagcttgatgacgactttcggcgccgtggcggaatggcggcttccatctctgaggcgagcgacgctatggatcccacagtggtttgctaagaaggccattttcaactctccactggaggctgctatggcgttccctcacctgcagcagcccagctttctactggctagcctgaaagctgactctataaataagccctttgcacagcagtgccaagacttggttaaagtcattgaggactttccagcaaaggagctgcacaccatcttcccatggctggtagaaagcatttttggcagcctagatggtgtcctcgttggctggaacctccgctgcttacaggggcgcgtgaatcctgtggagtacagcatcgtgatggaatttctcgaccctggtggcccaatgatgaagttggtttataagcttcaagctgaagactataagttcgactttcctgtctcctacttgcctggtcctgtgaaggcgtccatccaggagtgcatcctccctgacagtcctctgtaccacaacaaggtccagttcacccctactgggggccttggtctgaacttggccctgaatccgttcgagtattacatattcttctttgccttgagcctcatcactcagaagccacttcctgtgtccctccacgtccgtacttcagactgtgcctatttcatcctggtggacaggtacctgtcatggttcctgcccaccgaaggcagtgtgcccccaccactctcctccagcccaggggggaccagcccctcaccacctcccaggacaccagccataccctttgcttcctatggcctccaccacactagcctcctaaagcgacacatctctcatcagacgtctgtgaatgcagaccccgcctcccacgagatctggaggtcagaaactctgctccaggtttttgttgaaatgtggcttcatcactattccttggagatgtatcaaaaaatgcagtcccctcatgccaaggagtcgttcacgcctactgaggagcatgtgttggtggtgcgcctgctgctgaagcacctgcacgcctttgccaacagcctgaagccagagcaggcctcaccctccgcccactcccacgccaccagccccctggaggagttcaaacgggctgctgtcccgaggttcgtccagcagaaactctacctcttcttgcagcattgctttggccactggcccctggacgcatcgttcagagctgtcctggagatgtggctgagctacctgcagccgtggcggtacgcgcctgacaagcaggctccgggcagcgactcccagccccggtgtgtgtcggagaaatgggcaccctttgtccaggagaacctgctgatgtacaccaagttgtttgtgggctttctgaaccgcgcgctccgcacagacctggtcagccccaagcacgcgctcatggtgttccgagtggccaaagtctttgcccagcccaacctggctgagatgattcagaaaggtgagcagctattcctggagccagagctggtcatcccccaccgccagcaccgactcttcacggcccccacattcactgggagcttcctgtcaccctggccaccagcggtcactgatgcctccttcaaggtgaagagccacgtctacagcctggagggccaggactgcaagtacaccccgatgtttgggcccgaggcccgcaccctggtcctgcgcctcgctcagctcatcacacaggccaaacacacagccaagtccatctccgaccagtgtgcggagagcccggctggccactccttcctctcatggctgggctttagctccatggacaccaatggctcctacacagccaacgacctggacgagatggggcaagacagtgtccggaagacagatgaatacctggagaaggccctggagtacctgcgccagatattccggctcagcgaagcgcagctcaggcagttcacactcgccttgggcaccacccaggatgagaatggaaaaaagcaactccccgactgcatcgtgggtgaggacggactcatccttacgcccctggggcggtaccagatcatcaatgggctgcgaaggtttgaaattgagtaccagggggacccggagctgcagcccatccggagctatgagatcgccagcttggtccgcacactctttaggctgtcgtctgccatcaaccacagatttgcaggacagatggcggctctgtgttcccgggatgacttcctcggcagcttctgtcgctaccacctcacagaacctgggctggccagcaggcacctgctgagccctgtggggcggaggcaggtggccggccacacccgcggccccaggctcagcctgcgcttcctgggcagttaccggacgctggtctcgctgctgctggccttcttcgtggcctctctgttctgcgtcgggcccctcccatgcacgctgctgctcaccctgggctatgtcctctacgcctctgccatgacactgctgaccgagcgggggaagctgcaccagccctgaaggtgtcagctgccttcagagcaggctggagggatttgccacacagccccacccttgggctgagaggacctgggaagcccctccaggagggaacacggtcatcctcgggcttctggagcggggttcctgcagccgcagaggcatctggaggaaacgcaaccaagaaaggaaggcaggtgggccccagcaaaggagtagctgccagggctcaacagctacgctctgtgacagcgcagagctcagcggcggcctttccctccctccgccaaggactcacggccaagccagctctcggggccttttttccactgcccatttggctactctgctgcaccaagcttgggagccagcctgccaacagccacctgggcctggcctccccactggctggccttgaggttggcagagtgggttgtggcgcttcctctctctgtgtgggaccaggacagtggcttaagtctccactccaggaaagaatcaaagtttctagagttgtgagaaaaccagagagtggctgtcctgattcttcactgtgaggggcgttcttcatgttctcccagctgttccaagactgggccgtagaattccatgtttcaggagcctaagaccctcccagagcccaggggcttcaccgcagaccccaagccattgagcacatcacccaaagcagtggccaacatcgcggacccctgtgccttgtcacagatgggtgctggtcctcaggcgttggggacactgctgggtcgatggggtcggattctgccagtttctgctctgcagccaaagatggtcagaagcattgtcacttcagtaacatcaagtgctcaaagacatggcaaccgttcagtggtacttaagtattcaaaatatacaactacagattctctgacagaaaccagcacggggtcttcaccttcattcaccccacaggcgacatgcgagggagaacagcatctcagtggtgatttccaaaccaagcctttgttttcggtgtggggttttgggggtttgctttaatgtttttgaaattgtaaatgttgggctttgtattttgatgtaaactgagcataatggcattttagggcctgtgaccaaaaatgaagcttgtaacgaccatggatctgaataaacatgtccttgcttctgag

SEQ ID NO: 13 - protein, 473 aa, Homo sapiens
MATATEQWVLVEMVQALYEAPAYHLILEGILILWIIRLLFSKTYKLQERSDLTVKEKEELIEEWQPEPLVPPVPKDHPALNYNIVSGPPSHKTVVNGKECINFASFNFLGLLDNPRVKAAALASLKKYGVGTCGPRGFYGTFDVHLDLEDRLAKFMKTEEAIIYSYGFATIASAIPAYSKRGDIVFVDRAACFAIQKGLQASRSDIKLFKHNDMADLERLLKEQEIEDQKNPRKARVTRRFIVVEGLYMNTGTICPLPELVKLKYKYKARIFLEESLSFGVLGEHGRGVTEHYGINIDDIDLISANMENALASIGGFCCGRSFVIDHQRLSGQGYCFSASLPPLLAAAAIEALNIMEENPGIFAVLKEKCGQIHKALQGISGLKVVGESLSPAFHLQLEESTGSREQDVRLLQEIVDQCMNRSIALTQARYLEKEEKCLPPPSIRVVVTVEQTEEELERAASTIKEVAQAVLL

SEQ ID NO: 14 - DNA, 2780 bp, Homo sapiens
gcgcttgtgacccgccttccggaaggaagcggctaactatggcgaccgccacggagcagtgggttctggtggagatggtacaggcgctttacgaggctcctgcttaccatcttattttggaagggattctgatcctctggataatcagacttcttttctctaagacttacaaattacaagaacgatctgatcttacagtcaaggaaaaagaagaactgattgaagagtggcaaccagaacctcttgttcctcctgtcccaaaagaccatcctgctctcaactacaacatcgtttcaggccctccaagccacaaaactgtggtgaatggaaaagaatgtataaacttcgcctcatttaattttcttggattgttggataaccctagggttaaggcagcagctttagcatctctaaagaagtatggcgtggggacttgtggacccagaggattttatggcacatttgatgttcatttggatttggaagaccgcctggcaaaatttatgaagacagaagaagccattatatactcatatggatttgccaccatagccagtgctattcctgcttactctaaaagaggggacattgtttttgtagatagagctgcctgctttgctattcagaaaggattacaggcatcccgtagtgacattaagttatttaagcataatgacatggctgacctcgagcgactactaaaagaacaagagatcgaagatcaaaagaatcctcgcaaggctcgtgtaactcggcgtttcattgtagtagaaggattgtatatgaatactggaactatttgtcctcttccagaattggttaagttaaaatacaaatacaaagcaagaatcttcctggaggaaagcctttcatttggagtcctaggagagcatggccgaggagtcactgaacactatggaatcaatattgatgatattgatcttatcagtgccaacatggagaatgcacttgcttctattggaggtttctgctgtggcaggtcttttgtaattgaccatcagcgactttccggccagggatactgcttttcagcttcgttacctcccctgttagctgctgcagcaattgaggccctcaacatcatggaagagaatccaggtatttttgcagtgttgaaggaaaagtgcggacaaattcataaagctttacaaggcatttctggattaaaagtggtgggggagtccctttctccagcctttcacctacaactggaagagagcactgggtctcgcgagcaagatgtcagactgcttcaggaaattgtagatcaatgcatgaacagaagtattgcattaactcaggcgcgctacttggagaaagaagagaagtgtctccctcctcccagcattcgggttgtggtcacggtggaacaaacagaggaagaactggagagagctgcgtccaccatcaaggaggtagcccaggccgtcctgctctaggcagagtcccgggaccatggcctcctgccacacaacacgcagagaggactcaagactcccgctggccatggagtggcctgaaagagagcaagaacatgtggatctttgataggattgttaccaaatggtgtcagtatggaccaattgtgtgaccatgagaaggatgcttattttttttaaaaagaaaacacatctaaaagcccaggaactgatttttttaagaggaaaactaatgacagtgtataactgatgtttaaattgtgcatttagtactatttaaatgttttcttatactagtattttatattcttttgttgtcgtttaaaactggagcttcagtgtctcttccctccctctaatagtaatggttcagtaagcactccttaactccttagtatttcatagaaaaatgactgcaacattaaagctaagaggaacacttcaacatatgtggtacaaatttatattgaagatctaaataaaccacgtattttccagtcttcgttgtgtgaagctaaatggtggctaaaaggaacactttttgtgtgattattataaactttgcattgtatttgaatcttagaacttttgtacacactaaatattgatgtcacaccatttctaatctgagcatccttagccagagaatattcattatacttcctaagtgagcaataatttaaatcagaagctattttattttaatgtaattaacctttctttacatttcttatgtgttcacctctaatctgttttaggaagagagttggttattatgttgatcccataatataaatcatatcctttatattttagaatatctcaaatgtattccttttttgtatggtgggtttgcctagggacgtgtaactacaggcttttactaagccaaggaaaaagagaatttttcttttcatcttacaaattccagatatctacaaaagatgtgaaagcactaaaaataccatttttaagcagtactttacctgttttttctttagcaaaccaggttatgtggtgtaaaggtttgttatacgtgccacaatatagcatataaatattatgccatcattccttctcttgttaaaggtagaagaataaaattgtgatttttataacctgtgcttattactcaaatggtcttcaacatctttttaaacaacacatactttttgaatgttcagtttctattttgcttgaggtattttgtacatatgtgccttgtgattgctgctgctttaaaggataaagtactctttgggggatgagtctggtttgttttgttttattttttaatgaaataaacctatattcctgatt

SEQ ID NO: 15 - protein, 562 aa, Homo sapiens
MRPEPGGCCCRRTVRANGCVANGEVRNGYVRSSAAAAAAAAAGQIHHVTQNGGLYKRPFNEAFEETPMLVAVLTYVGYGVLTLFGYLRDFLRYWRIEKCHHATEREEQKDFVSLYQDFENFYTRNLYMRIRDNWNRPICSVPGARVDIMERQSHDYNWSFKYTGNIIKGVINMGSYNYLGFARNTGSCQEAAAKVLEEYGAGVCSTRQEIGNLDKHEELEELVARFLGVEAAMAYGMGFATNSMNIPALVGKGCLILSDELNHASLVLGARLSGATIRIFKHNNMQSLEKLLKDAIVYGQPRTRRPWKKILILVEGIYSMEGSIVRLPEVIALKKKYKAYLYLDEAHSIGALGPTGRGVVEYFGLDPEDVDVMMGTFTKSFGASGGYIGGKKELIDYLRTHSHSAVYATSLSPPVVEQIITSMKCIMGQDGTSLGKECVQQLAENTRYFRRRLKEMGFIIYGNEDSPVVPLMLYMPAKIGAFGREMLKRNIGVVVVGFPATPIIESRARFCLSAAHTKEILDTALKEIDEVGDLLQLKYSRHRLVPLLDRPFDETTYEETED

SEQ ID NO: 16 - DNA, 7250 bp, Homo sapiens
ccttggccgagaccggtcctctgcggagagggccccgccctctgtgaaggcccgcccgggaattggcggcggcgctgcagccatttccggtttcggggaggtgggtggggtgcggagcgggacttggagcagccgccgccgctgccaccgcctacagagcctgccttgcgcctggtgctgccaggaagatgcggccggagcccggaggctgctgctgccgccgcacggtgcgggcgaatggctgcgtggcgaacggggaagtacggaacgggtacgtgaggagcagcgctgcagccgcagccgcagccgccgccggccagatccatcatgttacacaaaatggaggactatataaaagaccgtttaatgaagcttttgaagaaacaccaatgctggttgctgtgctcacgtatgtggggtatggcgtactcaccctctttggatatcttcgagatttcttgaggtattggagaattgaaaagtgtcaccatgcaacagaaagagaagaacaaaaggactttgtgtcattgtatcaagattttgaaaacttttatacaaggaatctgtacatgaggataagagacaactggaatcggccaatctgtagtgtgcctggagccagggtggacatcatggagagacagtctcatgattataactggtccttcaagtatacagggaatataataaagggtgttataaacatgggttcctacaactatcttggatttgcacggaatactggatcatgtcaagaagcagccgccaaagtccttgaggagtatggagctggagtgtgcagtactcggcaggaaattggaaacctggacaagcatgaagaactagaggagcttgtagcaaggttcttaggagtagaagctgctatggcgtatggcatgggatttgcaacgaattcaatgaacattcctgctcttgttggcaaaggttgcctgattctgagtgatgaactgaatcatgcatcactggttctgggagccagactgtcaggagcaaccattagaatcttcaaacacaacaatatgcaaagcctagagaagctattgaaagatgccattgtttatggtcagcctcggacacgaaggccctggaagaaaattctcatccttgtggaaggaatatatagcatggagggatctattgttcgtcttcctgaagtgattgccctcaagaagaaatacaaggcatacttgtatctggatgaggctcacagcattggcgccctgggccccacaggccggggtgtggtggagtactttggcctggatcccgaggatgtggatgttatgatgggaacgttcacaaagagttttggtgcttctggaggatatattggaggcaagaaggagctgatagactacctgcgaacacattctcatagtgcagtgtatgccacgtcattgtcacctcctgtagtggagcagatcatcacctccatgaagtgcatcatggggcaggatggcaccagccttggtaaagagtgtgtacaacagttagctgaaaacaccaggtatttcaggagacgcctgaaagagatgggcttcatcatctatggaaatgaagactctccagtagtgcctttgatgctctacatgcctgccaaaattggcgcctttggacgggagatgctgaagcggaacatcggtgtcgttgtggttggatttcctgccaccccaattattgagtccagagccaggttttgcctgtcagcagctcataccaaagaaatacttgatactgctttaaaggagatagatgaagttggggacctattgcagctgaagtattcccgtcatcggttggtacctctactggacaggccctttgacgagacgacgtatgaagaaacagaagactgagcctttttggtgctccctcagaggaactctccctcacccaggacagcctgtggcctttgtgagccagttccaggaaccacacttctgtggccatctcacgtgaaagacattgcctcagctactgaaggtggccacctccactctaaatgacattttgtaaatagtaaaaaactgcttctaatccttcctttgctaaatctcacctttaaaaacgaaggtgactcactttgctttttcagtccattaaaaaaacattttattttgcaaccattctacttgtgaaatcacgctgaccctagcctgtctctggctaaccacacaggccattcccctctcccagcaccttgcagacttgggcccatcaagagctactgctggccctggctccgcagcctggatacttacctggccctcctccctagggagcaagtgccttccacttacttcccatccaggtctcagaggtctcaaggccaaccttggaatccttatttaaccattcaagtaatcaacggaagttttcaccctttaatcttaagtttagccttttaagaaaaacagtaagcgatgactgctgaaaggctcattgtgtaatctcccaagggtttggtcttattccattttcttctggtcaccagatgatttcttcctttaccatcaaatacttcttcataatggtcacagtctgaggatgtgcgcaaattctggttcttcccaagctctaaccgtaacacgtcccaccccctttttaaagcacttactgttttcagagcacccatatcccaccctggtgagaaggccactctcacatctgagtgttgggtacaaagctgctccgtagagtgatgtgcactcctggtgggtgaggggcaggggcagtggcagtgtgcaaagaattgattactccttgcagagcctgtggcttgcatttcctactgctttctacgtttgaaaattatgacagtctctggctaggtctgggtccagattaggatttaaactgataaaggaaactgttggtaaatcctctgctcagaaagcatttatcatgttcctatttaaggattaggtttattaatttaggcctcttagaagctaacccacttaaatattactcttctgaatgctagttctcttttattcttgatgtcctaagtcaattgaatctggcatctggggctagggtctgcctgtctacatattttttatttttttctgagaaattctgaacacatagatctctttcctaaactgacattttctattttgactgttttcatactataaccaggtaaagggacttctttcagagagctttatactgcctgaccaaagaacaaatctgaaaatcaccattttaaagttattttttcagttgaaccaaagtttaagtgaagaggacttttggcatattatacccaggatcagtttgtctttttgtatccatcaagtattacaggagaaggattgggaacagaatggaaaaacagtgtatgaaagtcatgttacaggccgagtgcggtggctcacacctgtaatcctagcactttgggaggctgaggcaggtggctcacttgaggtcaggaattcaagaccagcctggccaacatggtgaaaccccgtctctactaaaaagacaaaaaattagctgggcgtggtggcgggcacctataatcccacctacttggtaggctgaggcaggagaatcgcttgaacccaggaggcggaggttgcagtgagacgagattgtgccactgcactctagcctgggtgacagagcaaaactgtgtctcaaaaaaaaaagtcatgttacacatttaagtttttgaaattgctccttttatcggtaaagattctcaatccaaattctcctgggtgtgttgtcatcagctgtgatatgtttgtgcacattacgtatagcagaggatgtaagcaatattattgtttgtgaagttttgtttttaatgtcttgagtatgagttatgtttagtcactgtcagcatctgagaactttaataagcccttgagatattccaaagttttattttacttttttaaagaacagaaaaagatgaatgaaagaaccaaggagagatgcagagactatatttagcatgtataggttaaagtaagaaggaggttgtggtaactaaataggagtcctataaaatcaaatacattgtcaaccttttctgcacatctagtttcctaccatagaatcccactggaataccacatagcttttgcactgcagttactatttactaatgtaaacgtagggtttgtaaaagtcacaaacttataagcaatgaacttacctgctagtctttttattttggcttgcatgaagtcactgcaaattcaaatgtcagtaccggcatttaaaatatatctatatcactttgttggtacaaagttatttcaagataagtgtaattttgttacaagtttattttgaagagacaaatctcctgtgatctatgcaggacctctgtactttctaaagaacaaaatgttatgtagacattatacatggttggttgtctcttcttgaaactgtaatgtaaatctagggtccagtcatatcctaggtatcatcatttatccaagtacttggaggaatacaagtatatataaatacagtcattgagaataagtcgatttgaggcatacaagagtagtttcttacacagtttaacacggcctgattcaagactctgataggattcaaacagataccggttaaccatgactaccaaaactgatcatctgagtcgattgatagaggtgtgactagtccttagcactttttctcattcctctttttattcagcattgctgttacctatttcaggtttataagacctctttcagcagatcacatcagaagccaggaaatgcatagctaggagatgtcaaaagcccatatgaggagtggaccaagcagcagtggcggtttctcctcgcatctttttttttttaagctttaacttagcaggggcatggactttatagcactttttcaactttttgctttgctttggataagaaatccttacctttaaaaaaagcttctagtctccataacccccaaagtactgcttatttgtttgaagaatccagccatcgtagtgctttagtcactatcgtaaacattcatgatagggcaaggattttaaaacaggattcttgcttctgtagtcatcaaggtgaacagaagcatcctacacaaccactaagggctctatgtttgtgtcatgcctcttcaaacaccaaggagttgaacatgcttccagtgatttgtctccgtaatgccttcttcctttatttggcctttctttctttctgtaccttcaagttcttgatttttaaaattccaactctagagaaaaccaatatatggtggtgctgggctttgaagatagcatatcagacgccttggttctgtttgtacacttagccttacatttcaggaggaggcttttcattaggggcttaagctagctcctttggcttttaaaaaaaattttttttcaaatttcttcattacctaagggagcctgcatctaaatttctcaactagttcagcctagctgaattttctagtgtgtaatacactttgcttccttcttattggtgaaaaccagggggatgagtggcttccatggagagatttcctgatttctcagggaggaaaaaagtgatgacatttaccactacttttatgtttttcccctttttccaaattgataaggatttctggttcctagtgatccgggattgggcaacagtgcagaactgccagtcatgccgtaggccgtgaagaaagaatgtgagtaactgttgttttgcaaggatttgtagggttatgggcagttgttgtttgaagcattgctatgacctaattcccaaggtatctttcctctcttggtgttctaggtaagccaatgagctttaatctctacttgctataaccgtgtgcttagaaaaagaggtgagagtagtggttttccttcaaactgtccacattcatgaagattatgaattgttaggacagccagggcaagatagaccctgtctctacaaaaatttttttctaaattaaccgggcatggtggtgcctgcctgtagtcccacctgtgtgggagaatcacttgagcctgggaggtcaaggctgcagtgagccatgattgcacccctgcactccagcctgggtgacagagtgagaccctggctcaataagagggggaaaaaaaattgttaggagctgggtgcggatgcagcctgcaatcccagctacttgagaggctgaggccggaggattgcttaaacccaagaatttgagcgtagcctgggcaacacagcaagaccccatctaagaaaaaaatgttttttaaatcagcttagcccaaaggggttgtgaatggggaggtataaaaagcaaagattattttttggctactaagccaagaacttacagggattttttttttcagtcccagaacctacagataccctgctacttgcttcacgtggatgctcagtgcccagcagccatcttaatacattaaaccagtttaaaaaataccttccatgtggagaaaaacatgtctttttctcgcctcaactttatccacatgaaatgtgtgcccatggctgggcgcagtggctcacctgtaatcccaacactttgggaggctgaagcaggcagattgcttgaggccaggagttcgagaacagtctggccaacatggcgaaacctcatctctactaaaattacaaaaattagccgggcatggtggcacatgcctgtaatcccagctacgtcaggaggctgaggcacaggaattgcttgaacccaagaggcagaggatgcaatgagccaagatcacaccactgcactccagccttggcgacagagggagactctgtctcaaaaaaaaaaaaaaaaggtgtgcccaggcccctagccattgccatgtgcccagccagagagccaaattagagggctggcttccctatcacacagaataaatgctagtgctagccaatgatccctttgcttttaatgtatagaaaatactgttgttccttttgtcatttccagtgacatctgttttctaagcagctcttttctagggaggaaaccaaaggggctaggttaagaccctaatagaaatgttttttctaatctctggtgagtctggaagtgtcacattcacagtccacccttgggagtggcttggtggagctggggacaaggttttgtttactacatagtgcacatgataaatggccttaaactgtgattctttctggtaggataagttataataaactgaccctaaagaatgcaaaaaaaaaaaaaaaaaa

SEQ ID NO: 17 - protein, 552 aa, Homo sapiens
MANPGGGAVCNGKLHNHKKQSNGSQSRNCTKNGIVKEAQQNGKPHFYDKLIVESFEEAPLHVMVFTYMGYGIGTLFGYLRDFLRNWGIEKCNAAVERKEQKDFVPLYQDFENFYTRNLYMRIRDNWNRPICSAPGPLFDLMERVSDDYNWTFRFTGRVIKDVINMGSYNFLGLAAKYDESMRTIKDVLEVYGTGVASTRHEMGTLDKHKELEDLVAKFLNVEAAMVFGMGFATNSMNIPALVGKGCLILSDELNHTSLVLGARLSGATIRIFKHNNTQSLEKLLRDAVIYGQPRTRRAWKKILILVEGVYSMEGSIVHLPQIIALKKKYKAYLYIDEAHSIGAVGPTGRGVTEFFGLDPHEVDVLMGTFTKSFGASGGYIAGRKDLVDYLRVHSHSAVYASSMSPPIAEQIIRSLKLIMGLDGTTQGLQRVQQLAKNTRYFRQRLQEMGFIIYGNENASVVPLLLYMPGKVAAFARHMLEKKIGVVVVGFPATPLAEARARFCVSAAHTREMLDTVLEALDEMGDLLQLKYSRHKKSARPELYDETSFELED

SEQ ID NO: 18 - DNA, 3855 bp, Homo sapiens
agaaggagccagcatggacaatctcctttacagtttcggaagcaggtttgttgccatggagttcacattttgacgggagttgagaagtataaaggtaaccatttgttttagtttcaacgatctgacaaaaagataggctgttgctcttcttctggaaaagcctgattggtaagattcctttaagggctcagccccaaagagctttatcccatcccctcgcagactgaaaactaaagcctgcagagacctctgaaggaaaacctgtcccgggctctgtcacttcacacccatggctaaccctggaggtggtgctgtttgcaacgggaaacttcacaatcacaagaaacagagcaatggctcacaaagcagaaactgcacaaagaatggaatagtgaaggaagcccagcaaaatgggaagccacatttttatgataagctcattgttgaatcgtttgaggaagcaccccttcatgttatggttttcacttacatgggatatggaattggaaccctgtttggctatctcagagactttttaagaaactggggaatagaaaaatgcaacgcagctgtggaacgaaaagaacaaaaagattttgtgccactgtatcaagactttgaaaatttttatacaagaaacctttacatgcgaatcagagacaactggaaccggcccatctgcagtgccccagggcctctgtttgatttgatggagagggtatcagacgactataactggacgtttaggtttactggaagagtcatcaaagatgtcatcaacatgggctcctataacttccttggtcttgcagccaagtatgatgagtctatgaggacaataaaggatgttttagaggtgtatggcacaggcgtggccagcaccaggcatgaaatgggcaccttggataagcacaaggagttggaggaccttgtggctaagttcctgaatgtggaagcagctatggtctttgggatgggattcgcaactaactcaatgaatatcccagcattagttggaaagggatgcctcattttaagtgatgagttaaaccacacatcgcttgtgcttggggcccgactctcaggtgcaaccataagaatcttcaaacacaacaacacacaaagcctagagaagctcctgagagatgctgtcatctatggccagcctcgaacccgcagagcttggaaaaagattctcatcctggtggagggtgtctacagcatggaaggttccatcgtgcatctgccccagatcatagctctaaagaagaaatacaaggcttacctctacatagatgaagctcacagtattggggccgtgggcccaaccggccggggtgtcacggagttctttggactagaccctcatgaagttgatgtgctcatgggcacattcaccaaaagttttggagcttcaggaggttacatagctggaaggaaggacctcgtggattatttacgggttcactcgcatagtgctgtttatgcttcatccatgagcccaccgatagcagagcaaatcatcagatcactaaaacttatcatgggactggatgggaccactcaagggctgcagagagtacagcaacttgcgaaaaacacaagatacttcagacaaagactgcaggaaatgggattcattatctatggcaatgagaatgcttctgttgttcctctgcttctttatatgcctggtaaagtagcggcttttgcaaggcatatgctagagaaaaaaattggagtggtggtcgtgggatttccagccactcccctcgcagaagctcgggctcggttttgtgtttcagcggcacatacccgggagatgttagacacggttttagaagctcttgatgaaatgggtgatctcttgcaactgaaatattcccggcacaagaagtcagcacgtcctgagctctatgatgagacgagctttgaactcgaagattaagtttcctggtcctgaatgacacataaagactttgcgagaaagacctccctccttgcctcacaaggaatataaatggatttctcccccttcctcaggacaattttggttcccagaccagcttgattgaactgagggagacgttgttgtttttaatgtctccagcttggactgcagagacaaaaacatgattccagatttaagtctctcttcttccaagtattctactagaaatacacacacacacacacacacacttctgagaatatttttaatggcaataagcctgtgttttagctgctactgtgcagaccctttcagggattccaaccaaatgcaaatgagagaatttaaaatttttattcagtaagttcactatgtgtttacttattccatctgcagatgtaagtgagtgtggtggacagtagccaccttccttgttccactcataaaagcatcagctagatctcatctgtatcatgggaagttccaggcaggagggtaaagaaatgttgtttctaccatttgtcacatttggacgtttttcacagacaagtgtcaaaagacatagttaatgtttcgagggggaaagcagaactgatcaactgcgactagagacgtctttgaaggaaattttccttttcctcttgctggtctcctacagttttacagctgagcttttgaggtttggaaaattcaagatgtttgtttcataagaaaaggggcagaaagcaagcacaagacacttttaggtctagactaaaatggtagttacaacaatttgaaccttgttggtgcagtgcacggtgaatgcttaccctgcacagcctctattaccttaaggaattagttgtcattttcctattgaattttgagtaaatggtgaaattcagtgtcctttagaatcgactcccaagactatatttgaagaatgcattgattcaagaaggacatttaaaagcaaattctgactttttcaagaccaacacacttgtttaggcctattaaattgtactatcacttgttacatgccctctgaattgggagaaagtgggcttgcacactttgaggtaactaactgtaatttacttgtgttctctctttcttgctctccttctcaacatgaacacaaacctctatggaaaagtagcctcttgttaatctgatctagtttgtatggaaaaagcccatggagaaccttatctttaacaagctcccagagggtgcccagatttggaaattctagagagcagtggtgaccttttagcaaagcttctgtaacagttcgaatgagttgcagaacattccactccatcaaatgacactgtaaacaaatcacttcaagagaggcttggttttgtgtggcacagatggacccaagctttcatgctgtgcactgagatagaaactccacccgcagcgcctgcgatggatggagcagtgtgccctgatgctcaaagcgtattaaaggaaaaaaagtgatcagcatggaaagtttttatggggaaattataactccaagtgggtgcattggtttaaaaatggatcacaatgatagagttcttcatgaatgtttacaagttgtaaggaataccgttagtgaaagaggaaaagaagtgggttctgaaaatgcagtttcccatccaatgattttgataacaaaattccaacttttctcaatgaaactgatgcagtattatttgtgcaaataaaatgtgtcataaatatgcaaagaaagggagacatactgttcttatcttgaatatgtgcatttaaatgaattgtccaaaatgcataaatgccttgcgttctataaaggaaacaggaggtcaaaaagacagcgagatatctagtccttccacaaggtatggaaagcaataaacatcttcctttctttctgaaaaaaaaaa

SEQ ID NO: 19 - protein, 332 aa, Homo sapiens
MLLLAAAFLVAFVLLLYMVSPLISPKPLALPGAHVVVTGGSSGIGKCIAIECYKQGAFITLVARNEDKLLQAKKEIEMHSINDKQVVLCISVDVSQDYNQVENVIKQAQEKLGPVDMLVNCAGMAVSGKFEDLEVSTFERLMSINYLGSVYPSRAVITTMKERRVGRIVFVSSQAGQLGLFGFTAYSASKFAIRGLAEALQMEVKPYNVYITVAYPPDTDTPGFAEENRTKPLETRLISETTSVCKPEQVAKQIVKDAIQGNFNSSLGSDGYMLSALTCGMAPVTSITEGLQQVVTMGLFRTIALFYLGSFDSIVRRCMMQREKSENADKTA

SEQ ID NO: 20 - DNA, 5198 bp, Homo sapiens
tcttcccctccgccgcggcccgccccggcccgcaaacccaaacactccaggcgcccgcccgccgcgcgtgattctcgcctcgccgcagcccagccctgcgcgccttgcccggcggcccccgcccggccgctccgggcccctggccccgcggagcgatgctgctgctggctgccgccttcctcgtggccttcgtgctgctgctgtacatggtgtctccgctcatcagccccaagcccctcgccctgcccggggcgcatgtggtggttacaggaggttccagtggcatcgggaagtgcattgctatcgagtgctataaacaaggagcttttataactctggttgcacgaaatgaggataagctgctgcaggcaaagaaagaaattgaaatgcactctattaatgacaaacaggtggtgctttgcatatcagttgatgtatctcaagactataaccaagtagagaatgtcataaaacaagcacaggagaaactgggtccagtggacatgctggtaaattgtgcaggaatggcagtgtcaggaaaatttgaagatcttgaagttagtacctttgaaaggttaatgagcatcaattacctgggcagcgtgtaccccagccgggccgtgatcaccaccatgaaggagcgccgggtgggcaggatcgtgtttgtgtcctcccaggcaggacagttgggattattcggtttcacagcctactctgcatccaagtttgccataaggggattggcagaagctttgcagatggaggtgaagccatataatgtctacatcacagttgcttacccaccagacacagacacacctggctttgccgaagaaaacagaacaaagcctttggagactcgacttatttcagagaccacatctgtgtgcaaaccagaacaggtggccaaacaaattgttaaagatgccatacaaggaaatttcaacagttcccttggctcagatgggtacatgctctcggccctgacctgtgggatggctccagtaacttctattactgaggggctccagcaggtggtcaccatgggccttttccgcactattgctttgttttaccttggaagttttgacagcatagttcgtcgctgcatgatgcagagagaaaaatctgaaaatgcagacaaaactgcctaatcttcttaccccttggaagaagactgtttccaaataatttgaacagcttgctgctaaatgggacccaatttttggcctatagacacttatgtattgttttcgaatacgtcagattggaccagtgctcttcaggaatgtggctgcaagcaaggggctagaagttcacctcctgacagtattattaatactatgcaaatatggaataggagaccatttgattttctaggctttgtggtagagaggtgaaggtatgagaattaatagcgtgtgaacaaagtaaagaacaggattccagaatgatcattaaatttgtttctatttattcttttttgcccccctagagattaagtccagaaatgtactttctggcacataaagaaatcttgaggactttgtttaaaccttccataaaaaaacaattttcggtttctcgggttctctctctctctctctctgtctctctgtctctctgtctctctgtctctctgtctctctgtctctctctctctctctctttctttctttgtgtattttattcaagatgagttggacccattgccagtgagtctgaatgtcactgacagccctgtgttgtgctcaggactcactctgctgctggtggaaactcatggcttctctctctctttgatcccataaagctacgagggggacgggagagggcagtgcaatgggaagtaaagagatattttccagtaggaaaagcaatgctttcttgtctttagactcaaatgcttagggaacgtttcatttctcattcatggggaaaggcagcctccttaaatgttttctgaagagcggtaaaatctagaagcttaagaatttacagttccttcaataaccatgatgacctgaagttcacctatcccattttagcatctacttgtttttcccatctcttcctttccaattttgcttatactgctgtaatatttttgtaaaaaaaaaaaaaaaggaaaaaaaagaccagctaaaattttcgacttgactttttaacttaactcatgaattaattaaagcaaatgaaaaaattaaaaagtgtgactttttctcggagcatatatgtagcttttaggaaaggctgatgatggtataaagtttgctcattaagaaaaaaagacaaggctgattttgaagagagttgcttttgaaataaaatgatcacctgttctttatgtgactctcccactgaacctgcagacattatttttataccacatgctaaggaagcccactcatctaactctgtagccctggaaaccctcttggcccctgaatgttgtttccaggttataggatctgtgttcattagtggattttgacagcaagtgcctctgatgagttttagccaacattccccgttgcatcctgctcattgttcacactcctctgacttgaggagagctgccttcaccttgatcttgtaacctgtcttccttgcatatacggtagcacattccagatcacttcggtgagaaaagacctctgtttttgaagaggaaactttcttaaattgtaaaataaaacttaaaaaacataaaaactctatacaaaaattgaagacctaaaaattatagcataccttttttttgctcttgagtttttgaagctcttgactttaaatgacttaactttttaaaaaataagatgttttatggctttaacccatgtttttattaagatctgagaatagcatattttaaacactgatagatacagaagaaattattatttaatatataaattgcttggaatgatttcattgcagcttgtaaggatgggttgatgccagaatggtttcataaatctttcagtcttacagagccaaagtttgcagattatatcgagttgcgttgtgcagctgctaaaatggaatcatagctttttataatttgcagaaaacaattgcattaatgtctggatgtttctcagttttattcttttctcataaatgttcctcgaatgttggcaccttctactgctgatctatattagaatctagtccaagaaagtgtctgatttaataacattaaatagttatttcaatatattttttcaaattatcctcatagaacctgttgcctccagtgagatggtaatctactacccgaataaatttaagagcacctgttgagagcccctgttttgcggtagaagacaattgtccctatcccaatttccactgaaatatgagagcagatttgcagagaccatcatgtcagagaagaaatttccttgtcagagatgttaaagggctacagtcccttagcaaagaaacttcgaaatctaagttggtttgtgtttttctaaatttgataccgtaattcatttggcagcaaaatctgacttgaactgatgtgagagtattgtactttatatttatgctacttcattgtgacttaattttccctgaagaaatatccatggatttgattataggttttcccttagatgctgtggaggtgttttgtaatattcaaaatatgcccatattgcctttttaaaaccccaaagattatgaattctgaaacacatccagcccagcgggttttggataaggggttgtaggcatttaagcagcctcacataatgggctgacttcatccaaaacatgaaaatattacaaggcaaattctattttttatattttttggttcaatgcttgaacaacttgtttttctgctgggggaagaaaaaaagaaccaaccctgagtgtgattgttacggaaactaatgactttgtttttaaaggatcacattgattcaacaccttctattggacccagaagtgcgtaaatattacctatggtagtaaacgtttaattatcattcagtttaaatgttggccttctgtatgtagccaagaacagctcattttgtgaatttcagtttttaagtggctgctttttgatttggttgtattattttattataatgtatttgcaagtatattaaaaaattaacattgagccataaaaatccccaaatatgttcaaggacttcataattgaaaaatatatagaaaacaatccttacttctttttacaaaaacaaaatcatgggaattattcttttctatatatttagttataaatctttctctgggccgggcgtggtggctcacgccagtaatcccagcactttgggaggctgagacaggcgaatcacgaggtcaggagttcgagaccagcctggccaacatggtgaaaccccgtctctactgaaaatacaaaaaattagctggacacggtggcaggcgcctgtgtgtggcgggcgccagctactcaggaggctgaggcaggagaatcgcttgaacccaggaggcagaggttgcagtgagccaagattgcgccactgcactccagcctaggtgacagtgcgagactctgtctcaaaaaaaaaaaaaaagaaaaaaaatctttatctggatctgttaaaccatatattattgatcattgcaagtgaaattttgagagattgtttctagtatttaggtgatgaaaacatttggtaatattgctttggttcaaagaattttatgtctttatctttctagaagaaagcaattatatatatatttttgctaaattacataaacatttaattacatcaggttctaatttaaacatgtattactcacttgaggccacttttaaatattcatactctttgacataagatgctttgtatatttctcatttcttttagttcttagtaagtcagctttaaaaagtacctgccaaccagaaccttccatattctggactaaatcttgctcttcggattatacttcagtgcagtaactgtggatttgcaattttgaaggggagatagtagctattatattttacacttgcttgatgtgataactctaaagactttttaactgataaaagcgcacatggctattttgatacacaaagttgtgtttgctactttagaagcttttgtggcagaattgtaatctaattttcataccttgtatttctgaatcacaacaaaaaaataaatggggaacaagactta

SEQ ID NO: 21 - protein, 350 aa, Homo sapiens
MAAAGPAAGPTGPEPMPSYAQLVQRGWGSALAAARGCTDCGWGLARRGLAEHAHLAPPELLLLALGALGWTALRSAATARLFRPLAKRCCLQPRDAAKMPESAWKFLFYLGSWSYSAYLLFGTDYPFFHDPPSVFYDWTPGMAVPRDIAAAYLLQGSFYGHSIYATLYMDTWRKDSVVMLLHHVVTLILIVSSYAFRYHNVGILVLFLHDISDVQLEFTKLNIYFKSRGGSYHRLHALAADLGCLSFGFSWFWFRLYWFPLKVLYATSHCSLRTVPDIPFYFFFNALLLLLTLMNLYWFLYIVAFAAKVLTGQVHELKDLREYDTAEAQSLKPSKAEKPLRNGLVKDKRF

SEQ ID NO: 22 - DNA, 2499 bp, Homo sapiens
ggcggagggaaacagggcgctgtgagggcagccagtgaggacttgggctttccctgagtggaacaggagccatagagggctgcaagcagacgaggaacaggccaacagcgtcgcagccaccttctcacctctgtggggtgaggaggaagccaggataccagacggaagagggtagtggtggtagtggtggtggtggcggggagcggggggcaaggctgtgcagatgcagagaagtggccagatgcagggccacctggatgtgttgtgggggactcggtgtggggcaaggggaaggtgtgttcacctgtgcccctgaagttcaggtgcaggtgttcgggagccagcgcggctgctgcaggactcatccctgcatcctgcaccttgaaggccaggtgtggggacatcctgcgggctaggatggagggggggtgcagcagtgcctggggagatggcggtcctaacagaggtgaccagtgtcttcctcgatggccttcagtggctttgggcacagggccttgtcaatgaacagtgcattggaaagtcacttatggtggtcccaatgatgtcacagtcgatccagattgggagtccctcccacgttggcacccactcctgcagcctctctgcaactcatgtattaacctgatgttggggagccccccccccgggttccccagggtcacagcagcaagtgaccctcagagaaccccctaatctcagcagcccccatcacacctttgtcccagtagcccgctgtgcagacctgcagaagaagatgcctgggaagtgcccagggtacccgcgtccaagctggaaccccattgccaagctcctggtgtagggcagagagctgcgtctccactaggtgcgcctcccaacgccctggcgtgggccgtgtccttttgaagaatgagaatgtttcattgagcctacgcctcctgaatgcgggagggccacgggatggtcccaagactgtgccaaggcgtgccaggcctcgggtccttgagaaggagcgagggctccaagaacggtgaggtcggagcgggccggggcgatggagcgggagcggcgtggcgtgctggagggccgggagaagcgggggtcgccagtcgaggggactgcctgctagtgtcccccgtgccccccgtcacgggcacggcgcggtggggtgggggagcggggggcgcgcggggcaggcatgggggcgcgacacggcggtgcgcggggtctctcggggtccaggtccaacgggcccccagctccggcccccgcgcctgggtttctctgctgggaaacgggcaggggcgcgggcccgcctccagggcgccccgcgtccccgctggccgccccccagccgcgcccccagcggggcggagcttgcgccggtcccgcccctccgccctccgctctcccgcccgcgcgcccccggcccagctgcggcgcgtgacgcggggcgcgcggctccgtcggctaccgcgggcgggcgcaggcgacgggcacggcgggcgagcgggcggtatggcggcggcggggcccgcggcggggccgacggggcccgagcccatgccgagctacgcgcagctagtgcagcgcggctggggcagcgcgctggcggcggcgcggggctgcacggactgcggctgggggctggcgcgtcgcggcctggctgagcacgcgcacctggcgccgcccgagctgctgctgctggcgctcggcgcgctgggctggaccgccctgcgctccgcggccactgcgcgcctctttcgggtcagtgtggccgggggccgggacgaggggaccccggactgggggaagccgggaccggggacctggggccgccggcgcgttcctttcttccagcgctggctgctgcggatgaaggggtcgccggaacgtgggtgggggcctcgggctggagaggcccaggacgttggagccccaggataaggtctctcgggccgccgcgcgcccccttcagacagtgctgcgtgggggaaccgggtagatgcgggggtgtggccgggctggggtgagtctgggacatggggggcccgagacaggtgggatgtttgagacgtgggggaccgaggcacggccctttccggtcagtttgggaccagggggccccgcgcggtggatggctccggccaccgcgcgctctttccggcagggccgagccagacactgagaagcccaggaccgggaacgggagggctcggcgcaccgcggcctgcgccatgcgcctccctgccatgcgggctgcgccccggcccggagcgaggtccgctgcccgttttctgctgggtccgtgcggcggcggggccggcttgcccgctgtaatcgggaagaggcaggagctgcccggtcgctgccttgtgcctatctggggtcagcgccaccctccgaccagggatggcaagggcctgggtgctctggccacgccagtggtcgctcccttggaggtgacatggcccggggcttcagggttggactgtgctg

SEQ ID NO: 23 - protein, 380 aa, Homo sapiens
MLQTLYDYFWWERLWLPVNLTWADLEDRDGRVYAKASDLYITLPLALLFLIVRYFFELYVATPLAALLNIKEKTRLRAPPNATLEHFYLTSGKQPKQVEVELLSRQSGLSGRQVERWFRRRRNQDRPSLLKKFREASWRFTFYLIAFIAGMAVIVDKPWFYDMKKVWEGYPIQSTIPSQYWYYMIELSFYWSLLFSIASDVKRKDFKEQIIHHVATIILISFSWFANYIRAGTLIMALHDSSDYLLESAKMFNYAGWKNTCNNIFIVFAIVFIITRLVILPFWILHCTLVYPLELYPAFFGYYFFNSMMGVLQLLHIFWAYLILRMAHKFITGKLVEDERSDREETESSEGEEAAAGGGAKSRPLANGHPILNNNHRKND

SEQ ID NO: 24 - DNA, 2465 bp, Homo sapiens
gagcggagggttggggtctggcctcccgcgccggggcgaaggggcagccgcagcgcagaggcccgccccgccctcccctcccgtcacgcccagcctcccggcccttgggctgctcgcggcctttttttcccggctgggctcgggctcagctcgactgggctcggcgggcggcggcggcggcgccggcggctggcggaggagggagggcgagggcgggcgcgggccggcgggcgggcggaagagggaggagaggcgcggggagccaggcctcggggcctcggagcaaccacccgagcagacggagtacacggagcagcggccccggccccgccaacgctgccgccgggatgctccagaccttgtatgattacttctggtgggaacgtctgtggctgcctgtgaacttgacctgggccgatctagaagaccgagatggacgtgtctacgccaaagcctcagatctctatatcacgctgcccctggccttgctcttcctcatcgttcgatacttctttgagctgtacgtggctacaccactggctgccctcttgaacataaaggagaaaactcggctgcgggcacctcccaacgccaccttggaacatttctacctgaccagtggcaagcagcccaagcaggtggaagtagagcttttgtcccggcagagcgggctctctggccgccaggtagagcgttggttccgtcgccgccgcaaccaggaccggcccagtctcctcaagaagttccgagaagccagctggagattcacattttacctgattgccttcattgccggcatggccgtcattgtggataaaccctggttctatgacatgaagaaagtttgggagggatatcccatacagagcactatcccttcccagtattggtactacatgattgaactttccttctactggtccctgctcttcagcattgcctctgatgtcaagcgaaaggatttcaaggaacagatcatccaccatgtggccaccatcattctcatcagcttttcctggtttgccaattacatccgagctgggactctaatcatggctctgcatgactcttccgattacctgctggagtcagccaagatgtttaactacgcgggatggaagaacacctgcaacaacatcttcatcgtcttcgccattgtttttatcatcacccgactggtcatcctgcccttctggatcctgcattgcaccctggtgtacccactggagctctatcctgccttctttggctattacttcttcaattccatgatgggagttctacagctgctgcatatcttctgggcctacctcattttgcgcatggcccacaagttcataactggaaagctggtagaagatgaacgcagtgaccgggaagaaacagagagctcagagggggaggaggctgcagctgggggaggagcaaagagccggcccctagccaatggccaccccatcctcaataacaaccatcgtaagaatgactgaaccattattccagctgcctcccagattaatgcataaagccaaggaactaccccgctccctgcgctatagggtcactttaagctctggggaaaaaggagaaagtgagaggagagttctctgcatcctccctccttgcttgtcacccagttgcctttaaaccaaattctaaccagcctatccccaggtagggggacgttggttatattctgttagagggggacggtcgtattttcctccctacccgccaagtcatcctttctactgcttttgaggccctccctcagctctctgtgggtaggggttacaattcacattccttattctgagaatttggccccagctgtttgcctttgactccctgacctccagagccagggttgtgccttattgtcccatctgtgggcctcattctgccaaagctggaccaaggctaacctttctaagctccctaacttgggccagaaaccaaagctgagcttttaactttctccctctatgacacaaatgaattgagggtaggaggagggtgcacataacccttaccctacctctgccaaaaagtgggggctgtactggggactgctcggatgatctttcttagtgctacttctttcagctgtccctgtagcgacaggtctaagatctgactgcctcctcctttctctggcctcttcccccttccctcttctcttcagctaggctagctggtttggagtagaatggcaactaattctaatttttatttattaaatatttggggttttggttttaaagccagaattacggctagcacctagcatttcagcagagggaccattttagaccaaaatgtactgttaatgggttttttttaaaaattaaaagattaaataaaaaatattaaataaaacatggcaataagtgtcagactattaggaattgagaagggggatcaactaaataaacgaagagagtctttcttatgccttccttgca

SEQ ID NO: 25 - protein, 383 aa, Homo sapiens
MFWTFKEWFWLERFWLPPTIKWSDLEDHDGLVFVKPSHLYVTIPYAFLLLIIRRVFEKFVASPLAKSFGIKETVRKVTPNTVLENFFKHSTRQPLQTDIYGLAKKCNLTERQVERWFRSRRNQERPSRLKKFQEACWRFAFYLMITVAGIAFLYDKPWLYDLWEVWNGYPKQPLLPSQYWYYILEMSFYWSLLFRLGFDVKRKDFLAHIIHHLAAISLMSFSWCANYIRSGTLVMIVHDVADIWLESAKMFSYAGWTQTCNTLFFIFSTIFFISRLIVFPFWILYCTLILPMYHLEPFFSYIFLNLQLMILQVLHLYWGYYILKMLNRCIFMKSIQDVRSDDEDYEEEEEEEEEEATKGKEMDCLKNGLRAERHLIPNGQHGH

SEQ ID NO: 26 - DNA, 3894 bp, Homo sapiens
cgggatgcaccgctgcggggaccccccgcccggcctcgcggacccgcctcggcccaggacggaggaggaggtggtgaaacaggaagcaacatcccatcaggaagaacctgagggtgccatccagtagcttcgcctcacgtcactgactgccctcagtcagaaatcttgactcgcaactttggagggtgggtctctgaaggaatttcaggtcttacacagagatgagttgtgctgctctctgaggagaacgaagctggttggaacgttggaagctgctctctgactacacttcacaagcaaggggcaccttttgtggactgacatttcagaaagggatgttgtgaaacaaaagctgacatttatatatatatacatatatacagtatttgagttcctcagtagaaagctatcatatatactcagaatgttttggacgtttaaagaatggttctggttggaaagattctggcttcctccaacaataaagtggtcagatcttgaggatcacgatggactcgtctttgtaaaaccttctcatttatacgtgacaattccatatgcttttctcttgctgattatcagacgtgtatttgaaaaatttgttgcttcacctctagcaaaatcatttggcattaaagagacagttcgaaaggttacaccaaatactgtcttagagaattttttcaaacattccacaaggcaaccattgcaaactgatatttatggactggcaaagaagtgtaacttgacggagcgccaggtggaaagatggtttaggagtcggcggaatcaagagaggccttccaggctgaagaaattccaggaagcttgctggagatttgcattttacttaatgatcactgttgctggaattgcgtttctttatgataaaccttggctatatgacttatgggaggtttggaatggctatcccaaacagcccctgctgccatcccagtactggtactacattttagaaatgagtttttattggtctctgttatttagacttggctttgatgtcaagagaaaggattttctagctcatatcatccaccacctggctgctattagtctgatgagcttctcttggtgtgctaattatattcgcagtgggaccctcgtgatgattgtacacgatgtggctgacatttggctggagtctgctaagatgttttcttatgctggatggacgcagacctgtaacaccctgtttttcatcttctccaccatatttttcatcagccgcctcattgtttttcctttctggattttatattgcacgctgatcttgcctatgtatcacctcgagcctttcttttcatacatcttcctcaacctacagctcatgatcttgcaggtccttcacctttactggggttattacatcttgaagatgctcaacagatgtatattcatgaagagcatccaggatgtgaggagtgatgacgaggattatgaagaggaagaggaagaggaagaagaagaggctaccaaaggcaaagagatggattgtttaaagaacggcctcagggctgagaggcacctcattcccaatggccagcatggccattagctggaagcctacaggactcccatggcacagcatgctgcaagtactgttggcagcctggcttccaggccccacaccgaccccacattctgcccttccctctttctcaccaccgccttccctcccacctaagatgtgtttaccaaaatgttgttaacttgtgttaaaatgttaaatataagcatgcccatggatttttactgcagttaggactcagactggtcaaagatttcaaagatttctccacagaaccgtctcagttctaattgcactccctcatgcatgtcactttctcaggggctcgctttgttatagaccctttcgcctcgccaccttgcctgtcctcaggacgctttcacaggtgctaagtgatctcatttttcccaggtgtgtttggccacaaagagcagcttctttctcaaaatgagttagaagtggcagtgggacaggagcggaaggaccacaccaggagacacttccatcctgaagcttaggtgcctcatctccacagggcggtggcagtccctgcctgccaccccacagggttacagcaaggattaagtgagatcacagaagtttaagtacacactgtcaaccgtggggtcatcgtgaaccaacccactttgcactgtttgggagacagaaacctggctaaacatggcactgcaatgggcctgaacaaaaggcagaatctttaaaaattcctcttaaatgactctggagatacctggaaatgaaagtgccaagaaaggtgttccattttatttgctaactatttatgcattagctccccaagggtcattctcagattctcctggaattcttctccctcaggacccatggttcaaggaaggaagcttaggccctgttccttcctgtcctttgctggtctttcccttttttcctttctaggagggaagcttctgtgctgctgccctgagcccttccttcaggccagcacagtacctggggacctccacgggggaatgggatccaggccaggttgcttgctgagcctcatcacccaggaggcctgagcctctggggagggcacgcatgtacactgccagacccaggggagatcttgggaacagaggatgctacgtgatttcctctggcttccaacccaatcagcctgcatcacagtgaaacacaacacaaaaggccataaaaggcatacccctgagaaatgtttaagggcaggtgctagagtcagtgccagctccctgggaggagggatagggacgggcagatcagtagccagcttgtcctcaccctccggaagggagcaccggagaagacttgcacacgtccctgcctcccttgcggccttgtcaccagagggacacatctcctgggcacaatgtgcagggctgacctgggagacttctcaggtggctgctggctgaggagaggccaggccttcccaaggaagaccctgagtaaaatctgcatctgtcctcaccacctggcacagtctctcataacggtcagattttgtatgttcatcctatttattcaggggctcgtactagaaagccacaggagaggtcggcttgtagggactggaaaatcagccccaagcagagcagctgcagggcccctggagccggaaggacactgcacagaacagactgcgttattgttatttttaaataaaaatatacatttgaaaccttaaggctaaacaaaaataaacaaaaaatcctttagaattctttccacaacaatatctctttctgagaaattgttacaaacaaggtcagattttctctgtataacatttgcttttatgaggacaatatcatatgcattatatgcataatatgatattataaatcaaaatgcctgcacccactttagggtatagctattgacttattattaatattatattattattattttgctggaagaaggtcacactaagatataattttttatgttttcagttaacggtatgctttcttctttgcttatttggtttttgtctctgtaccaaatatcttcttgcttaaggtagaaaagtatttgtttacctctatctccagttttttttcttatttgaatgttgaaggtaaaattgatataccaattttaactatttctgatacagctgaaagcactaaactacttcataagaagtagatactcatttttgtaacactatttagggcttttgtggttaattttaaaggaaaccactctttctacaggaaacaagggctcaggattcttcagatgaccttataaaaatgcagtccacagtgctatcaatattgtaacagtaatgactacaataaagccaaaagtccagtgta

SEQ ID NO: 27 - protein, 394 aa, Homo sapiens
MLSSFNEWFWQDRFWLPPNVTWTELEDRDGRVYPHPQDLLAALPLALVLLAMRLAFERFIGLPLSRWLGVRDQTRRQVKPNATLEKHFLTEGHRPKEPQLSLLAAQCGLTLQQTQRWFRRRRNQDRPQLTKKFCEASWRFLFYLSSFVGGLSVLYHESWLWAPVMCWDRYPNQTLKPSLYWWYLLELGFYLSLLIRLPFDVKRKDFKEQVIHHFVAVILMTFSYSANLLRIGSLVLLLHDSSDYLLEACKMVNYMQYQQVCDALFLIFSFVFFYTRLVLFPTQILYTTYYESISNRGPFFGYYFFNGLLMLLQLLHVFWSCLILRMLYSFMKKGQMEKDIRSDVEESDSSEEAAAAQEPLQLKNGAAGGPRPAPTDGPRSRVAGRLTNRHTTAT

SEQ ID NO: 28 - DNA, 1817 bp, Homo sapiens
aagccttttttcccctgctgggggccgaggcccggccaggagcagagtccggctgcctggggcgggcggcgcgtgtctgcagctgctccgggtagcccgctaggcgcgccgtccccagccccgccgccggccctcggtgcgcccggccgcctgcacccccaggagcagctgctgtgaataaacacagaagtggagctgggggactgattagaagcctcattcagtgcacctgggccccagcaggcccagccaggcgtggaggaagaggcattgaggactttccttacctgtttttccagctcacccactgccagcagagaatgctgtccagtttcaacgagtggttttggcaggacaggttctggttaccacccaatgtcacgtggacagagctagaagaccgggatggccgtgtctacccccacccccaggacttgttggcagccctgcccctggcgctggtcctcctggccatgcgccttgcctttgagagattcattggcctgcccctgagccggtggctgggtgtgagggatcagaccaggaggcaagtgaagcccaacgccacgctggagaaacacttcctcacggaagggcacaggcccaaggagccccagctgtctctcctggccgcccagtgtggcctcacgctgcagcagacccagcgatggttccggagacgccggaaccaggatcgaccccagctgaccaagaagttctgtgaggccagctggaggtttctcttctacctgtcctccttcgtgggcggcctctcggtcctgtaccacgagtcatggctgtgggcaccagtaatgtgctgggacaggtacccaaaccagactctgaagccatccctgtactggtggtacctcttggagctgggtttctacctctcactgctaatcaggctgccctttgatgtcaagcgcaaggatttcaaggagcaggtgatacaccacttcgtggcggtcatcctgatgaccttctcctacagtgccaacctgctgcgcattggctctctggtgctgctgttacacgattcctctgactacctgctggaggcctgtaagatggtcaactacatgcagtatcagcaagtgtgcgacgctctcttcctcatcttctcctttgtcttcttctacacccgactggtcctctttcccacccagatcctctacaccacatactacgagtccatcagcaacaggggccccttcttcggctactacttcttcaacgggcttctgatgttgctgcagctgctgcacgtgttctggtcttgcctcattctgcgcatgctctatagcttcatgaagaagggccagatggagaaggacattcgtagtgatgtagaagaatcagactccagtgaggaggcggcggcggcccaggaacctctgcagctaaagaacggggcagctggagggcccaggccagcccccactgatggccctcggagccgggtggccgggcgtctgaccaacaggcacacaacagccacatagccgggcggggctggctgtaaggggttgcccccccgccagtgccttggatatttctggggtgactggactggcgcccctgggccacctttctggagacagggagggccccacccggggtgggtgggaaggctgatgatctgtctccagccccttccttctgcccacccacccttcttccctctgggcaactggacagatctgggagccagcagctggatgctgtggctggccagagacacctccaggctgtggcctgggggctggggggagccccaggctgaaaagggtccaattaaaacaaatggagccaaa

SEQ ID NO: 29 - protein, 392 aa, Homo sapiens
MATAAQGPLSLLWGWLWSERFWLPENVSWADLEGPADGYGYPRGRHILSVFPLAAGIFFVRLLFERFIAKPCALCIGIEDSGPYQAQPNAILEKVFISITKYPDKKRLEGLSKQLDWNVRKIQCWFRHRRNQDKPPTLTKFCESMWRFTFYLCIFCYGIRFLWSSPWFWDIRQCWHNYPFQPLSSGLYHYYIMELAFYWSLMFSQFTDIKRKDFLIMFVHHLVTIGLISFSYINNMVRVGTLIMCLHDVSDFLLEAAKLANYAKYQRLCDTLFVIFSAVFMVTRLGIYPFWILNTTLFESWEIIGPYASWWLLNGLLLTLQLLHVIWSYLIARIALKALIRGKVSKDDRSDVESSSEEEDVTTCTKSPCDSSSSNGANRVNGHMGGSYWAEE

SEQ ID NO: 30 - DNA, 1986 bp, Homo sapiens
ttcggggtgggcgtaagatggcgacagcagcgcagggacccctaagcttgctgtggggctggctgtggagcgagcgcttctggctacccgagaacgtgagctgggctgatctggaggggccggccgacggctacggttacccccgcggccggcacatcctctcggtgttcccgctggcggcgggcatcttcttcgtgaggctgctcttcgagcgatttattgccaaaccctgtgcactctgtattggcatcgaggacagtggtccttatcaggcccaacccaatgccatccttgaaaaggtgttcatatctattaccaagtatcctgataagaaaaggctggagggcctgtcaaagcagctggattggaatgtccgaaaaatccaatgctggtttcgccatcggaggaatcaggacaagcccccaacgcttactaaattctgtgaaagcatgtggagattcacattttatttatgtatattctgctatggaattagatttctctggtcgtcaccttggttctgggacatccgacagtgctggcataactatccatttcagcctctttcaagtgggctttatcactattatatcatggaattggccttctattggtcccttatgttttctcagtttacagacattaaaagaaaggacttcctgatcatgtttgtgcatcacttggtcaccattgggcttatctccttctcctacatcaacaatatggttcgagtgggaactctgatcatgtgtctacatgatgtctcagacttcttgctggaggcagccaaactggccaattatgccaagtatcagcggctctgtgacaccctttttgtgatcttcagtgctgtttttatggttacacgactaggaatctatccattctggattctgaacacgaccctctttgagagttgggagataatcgggccttatgcttcatggtggctcctcaatggcctgctgctgaccctacagcttctgcatgtcatctggtcctacctaattgcacggattgctttgaaagccttgatcaggggaaaggtatcgaaggatgatcgcagtgatgtggagagcagctcagaggaagaagatgtgaccacctgcacaaaaagtccctgtgacagtagctccagcaatggtgccaatcgggtgaatggtcacatgggaggcagctactgggctgaagagtaaggtggttgctatagggacttcagcacacatggacttgtagggccactggcaacatactcctcttggcccttcccatatctactcttctgtgattgggagactgcaaggcactgaggagtatcaaagaagcaaatattttcactttgaaagaaaactgccattttgtatttaatagcctccaggttctttcagtaatgttatttgctctgtgtgtttttgtgtgtttgttgatgtgcgtttgtgcatatgcgtgagtttcattgccggggttggggcacaattgtggactggggccatgaggccttccctggtccccactgaacccaccttagttccacatttggctgcatcttgaattatgccaactccagacttctccttcttttttgcccttggctcttgacactctaaacccctggaccatctgaatggagcagccaagttcagtcccacatttctgtactgttcctctttcacagctggaatatgtcacatgatgaagttgtatagaaacagaaccatggatggatggccaggattgccgtggtccctagctagatccccttcctatcaatcacctgatagcaacagggacagctgccaataccctgctctttactcaatggtacccagggagggagcatgggaagagggtgagctgagggctggaggagggcaacagccactgggtgagctgttcacggtcttatactattgtttgtttgtgattaaaagtgcttcaacccaaaaaaaaaaaaaaaaaaaaaaaaaaaaaaaaaaaaaaaaaaaa

SEQ ID NO: 31 - protein, 384 aa, Homo sapiens
MAGILAWFWNERFWLPHNVTWADLKNTEEATFPQAEDLYLAFPLAFCIFMVRLIFERFVAKPCAIALNIQANGPQIAPPNAILEKVFTAITKHPDEKRLEGLSKQLDWDVRSIQRWFRQRRNQEKPSTLTRFCESMWRFSFYLYVFTYGVRFLKKTPWLWNTRHCWYNYPYQPLTTDLHYYYILELSFYWSLMFSQFTDIKRKDFGIMFLHHLVSIFLITFSYVNNMARVGTLVLCLHDSADALLEAAKMANYAKFQKMCDLLFVMFAVVFITTRLGIFPLWVLNTTLFESWEIVGPYPSWWVFNLLLLLVQGLNCFWSYLIVKIACKAVSRGKVSKDDRSDIESSSDEEDSEPPGKNPHTATTTNGTSGTNGYLLTGSCSMDD

SEQ ID NO: 32 - DNA, 6259 bp, Homo sapiens
gggcgggagcagcggcggcggcggcacaggctcggggccagccgggcgcgcatccccgggcgccctgcgcggtggagagcttggcgggctgcgggtgccgcaggacaggagtggacaaagcaagatggcagggatcttagcctggttctggaacgagaggttttggctcccgcacaatgtcacctgggcggacctgaagaacacggaggaggccaccttcccgcaggctgaggacctctatctcgcttttcccctggccttctgtatcttcatggtgcggctcatcttcgagagatttgtagccaaaccgtgcgccatagccctcaacattcaggccaatggaccacaaattgctccgcccaatgccattctggaaaaggtcttcactgcaattacaaagcatcctgatgaaaagagattggaaggcctctccaagcaactggactgggatgttcgaagcattcagcgctggtttcgacaaagacgcaatcaggagaagccaagcacgctgacgaggttctgtgagagcatgtggagattttcattttacctttatgtatttacctacggagtcagattcctgaaaaagaccccctggttgtggaatacgaggcattgctggtacaactacccctatcagccactcacaactgaccttcactactattacatcctggagctgtcgttttattggtctttgatgttttctcagttcactgatatcaaaagaaaggactttggcattatgttcctgcaccaccttgtatctattttcttgattaccttttcatatgtcaacaatatggcccgagtaggaacgctggtcctttgtcttcatgattcagctgatgctcttctggaggctgccaaaatggcaaattatgccaagtttcagaaaatgtgtgatctcctgtttgttatgtttgccgtggtttttatcaccacacgactgggtatatttcctctctgggtgttaaataccacattatttgaaagctgggagatcgttggaccttacccttcctggtgggtttttaacctactgctattgctagtacaagggttgaactgcttctggtcttacttgattgtgaaaatagcttgcaaagctgtttcaagaggcaaggtgtccaaggatgatcgaagtgatattgagtctagctcagatgaggaggactcagaacctccgggaaagaatccccacactgcgacaaccaccaatgggaccagtggtaccaacgggtatctcctgactggctcctgctccatggatgattaattactcaaaactacaagtcccaagcaaagtgaactatttgttcctggaagtatttaataagttgcaaatgcagttcctttcataatatctcagcaccagaaacaaaaattaagattatcaaagcattttgaatagtgcactgccatgtgtcctgtctgtgaatgaagaagaattaccattctctctttgtaggcatgctgtatgtaattgacacaagggaacagtatttgcatttgtactgtcttagaatattatttatttttttgtatttgtaaatctgtggacaaaagagggtttcctcactccttttactcactgggctcatgacagtgaaggagatgctccatctgcttctccccctttctcttgctgtagtccaatgtgctatgagcatcagcttactttgtcacttagagcaagcaaaacccagtgcaagagtctcgttcagctctaaataggtttgctttcttttagttacagtgcccattttgaaattgcctatacagtcttagtgaccatttaaaccggacgaactaggtgtttaattttcactcttcatgttcaattagcagttcaaattaaagaagatggttattggagaacttttttgaatggttttgtattaaattgctttgaaatagatttcatttcttgtgcacacagccaagatttcttcaatgggtgtgagctagttgagggttaaccttgtaggttgcagagtgtatttgtttgtttgtttgtttttctctgtgatgaggtcagtgctctgattttgaaggaggatattcactgaagctcatagttataaacaaggaaatcactgttaagaatgggaatttgtcctgtgttctgggaataacataaagagagcaactgatttcagccaggttttgccactaccctataattagtgcagtcttatgttataaaagaaagaagttaactatatttggggacaaaaaaatatttcaagagttgataaagattacctgtgcagtgcagagcactttaatgcaaccagctttcaagaaaaagccctatctagtacttgatgttgatgtttttattttgctgagcaaaataaagccaatgggagaaagactattttaccctttgcttttctccttaaacgtaatccagatgactttcctgttactaaacactgagcagcattacactacaatgcttctttggtttccaggaatttttttcaaatggggctgtttctggaaaaatgaaaaattctattggacaatggcaatatcaacaatgaggaaaattactgaagaataagtttccataagtctcctacatagcagtgttatttatgtacagataagaaaaccatatgtcagccaaagattttatctcttcttctaacttttagtaagaggaaaaagggattataaaacccttcataaatcaagaaggccatcacttagaacgaaccccaaacaaaaatgccataatataaatgtgtgaatcagggctgtgaagacaacagcagaaatgctaacaagcgtgcagaaacaccagagagtgcgtatcctgctcagaaccattcacatttaattcaattcttggaaaaaattaaagctttttgcccacaatttgcaatctgtgggttaatagttaaaagaatgttcccaaccaaaaaattcttaccgtaatattatatcttgccctacttatttacaaaataatatgtttctgttatggtccttagtaataattgaagaggcttagaaatacatctgcttgtttattgagaaaacgatgcaaataattctgcttttagagccttgttattttatttcacaaaacaggcatatgtctaggagtgtaatttgtggatggttgagtttgtaagaacaatcataaaaggacttgttagtctccagaacatctgctaaaatgcaagtatatgtataaggtaatagcatattacagctgaaaattttgagaaggtaaaagtttcttaattaaaatatgaacatatttagcttgctttagtgtctggggcaagtcctctcaatggctaaaattaactttagagatccatgtgttcaggtttagatcatatgacactcgagcacagaagaataatttcaaggaggtcatcttgtaaattaaaggtttagaagaattgcataaaacgtagtaaatggggtctgtcattagcaaaggcaaatctaagcaatcatttttccccccagaagttacttagaaggagaactgggaacacttggggtctctctaactgatggcattcacttcacacagtcgtctatgttatccagagatttttatttcattttacattttagggcacagttctttggggctaattaaaatggggtttgcaggctttttatggtgaagaataatatatctctgtctatagctttcccatggtagcctgataaggctgagagagaaaaatatgtgcagtatctcatcctccccctgtaccaggccatagctttgaagtgtattttgtaaattcaactataggttagtcagaatgctgtttttcgttaattaacttagcctgtgttgatatctcctccttcctggtcacattcaaaccttcccagagtacaaaggggtatgtagaaaggattccagaagaagtaatactttattctctaatgttaatagcttttctggatctcttagtagggggaaagtagaaaatcgagtagaatttggcctcaggtcaataaatgatataaaaacatgtgttctatttatgtatatatatgtatgtttctccaaaaagtgataaaaccaaaatatcactgactcatccctacccatattcttttcataaaacccactcaccaggtacaatagaaacttccctccttgtttgtcagcctcctgttcatgttccccacacacctgaaggtggtagaatcttttcagcctcttagccagtgagctaaatatggctaagcacaggtcataagagcacctaaggccagcatataagccaactacagttcacctttccaaatttggtcctatggatgttgagcatagggaagcaactctcagtattttggattattcaagtgtatgtggtaaaaatgcagatgattgctgctttaccccagggtttattagcatcctacttctgctgggctgcatcattatataatgccacaggcatctagtcaaggtaaagaagccagaaaggttaggcaagaagtgagataaaatcagatcacctttgatcaaaatggttggtgaacctccacatgtccagttctgttgccaaactttccattcagagtatttggtggagtttgaatttgagcaaactaaatgccttcatcttaggtagaaagggcctgaatcttccattttatattcaaacctcattgttatttggcctaagtaaaaagtcagatttcatttccatttacctgagttcgctttaaagagcttttcaaagagagctttatagacacccacaattgtccccaatctcttcatgatgttgcattaatagttgtttttgtccctttcttggaaatgttaatgccaaagttgcctgaacattgggcggttttcttaatttgaagtataaaaattataaagagtaattccaaaggtattaaaagattgtttaacagtatgtgtggtgatgtcattatctccagagaggcttcaagaaatcctttggaaataaaaagttaaatgtttacatttcatgtggtatttcaggtcttcaggttctgattagcttacttttttcctttgtctttggctgatttctgctttgtagataaataataatagccctgagatgtttctaacatttaaataagaaaaaaatcaaatcgaagtcagcctgctggaaaagtgatcacatggcagttgcagtaacttgtatggaaagagaaaatgcaatgagcccagttactgcacttgccactaccatgctgtccatggaaggaataatcagcagttcagttgtcacaagccgcccttgaagaaaacgcagcaaaatattttaaaatgaagatattgcagtccccagagccagtgaaggtttcttttggtaaaatgaaattgtgccattgtcaaagtaccccgtagtgatgagcactgactggttcactggccacattttagttcttcataataataggccacaaaagggctctgtggtttgcctccatgtgcactggcccctccccacccctagggggcactcagtagctgctgagaaggcctgtccacgaggctgttggaacccctccaataaatacttagaggtagtgtatctgatgcttgttttcgtggagaaaattgtattggagaacttaaaacatcacgaatatttttaataggatccgcagacacccaaaggagaagcttggtcttttccaggtatttccaacttgagttcaacccaaagcctttgaaaggaatgcattaccacatgaccacatgctgagaccccatggggtctaacacgggacctaagaaagtctctgcagccagatagtacatggtgtctccacaaaactaggcattctggagattgcccagaaagggatgtgaggggaccgttaagatctgtcttgcttatctcatgcactcacattccttcagcctcctggagttcctgataaaaggaagccagggtgttgacattttttagctattgatttcccaatagcttgtggatcagttgtacacccacacttccttctctgcctaattccgtttttctggaaaaagtagtatgcccatgtatgtgtgtttttcttaacacaggtccatgaaagtttggcttcctggtttgatgtctgttgcgtggcctggaaaccagggagcagcaactattgagatggtttctgtgttcagtgaaaaattctatttcattgagacaattttttctttatccacagtaattttttgacactgtcatcatgaaactacccttaggaaaataagattacctgc

SEQ ID NO: 33 - protein, 323 aa, Homo sapiens
MGSRVSREDFEWVYTDQPHADRRREILAKYPEIKSLMKPDPNLIWIIIMMVLTQLGAFYIVKDLDWKWVIFGAYAFGSCINHSMTLAIHEIAHNAAFGNCKAMWNRWFGMFANLPIGIPYSISFKRYHMDHHRYLGADGVDVDIPTDFEGWFFCTAFRKFIWVILQPLFYAFRPLFINPKPITYLEVINTVAQVTFDILIYYFLGIKSLVYMLAASLLGLGLHPISGHFIAEHYMFLKGHETYSYYGPLNLLTFNVGYHNEHHDFPNIPGKSLPLVRKIAAEYYDNLPHYNSWIKVLYDFVMDDTISPYSRMKRHQKGEMVLE

SEQ ID NO: 34 - DNA, 1375 bp, Homo sapiens
  misc_feature (1246)..(1246): n is a, c, g, t or u
gccgccgccacctctgagcagccggctgggagcgagagccgacagctagtctgcaagccaccgctgtcgccatggggagccgcgtctcgcgggaagacttcgagtgggtctacaccgaccagccgcacgccgaccggcgccgggagatcctggcaaagtatccagagataaagtccttgatgaaacctgatcccaatttgatatggattataattatgatggttctcacccagttgggtgcattttacatagtaaaagacttggactggaaatgggtcatatttggggcctatgcgtttggcagttgcattaaccactcaatgactctggctattcatgagattgcccacaatgctgcctttggcaactgcaaagcaatgtggaatcgctggtttggaatgtttgctaatcttcctattgggattccatattcaatttcctttaagaggtatcacatggatcatcatcggtaccttggagctgatggcgtcgatgtagatattcctaccgattttgagggctggttcttctgtaccgctttcagaaagtttatatgggttattcttcagcctctcttttatgcctttcgacctctgttcatcaaccccaaaccaattacgtatctggaagttatcaataccgtggcacaggtcacttttgacattttaatttattactttttgggaattaaatccttagtctacatgttggcagcatctttacttggcctgggtttgcacccaatttctggacattttatagctgagcattacatgttcttaaagggtcatgaaacttactcatattatgggcctctgaatttacttaccttcaatgtgggttatcataatgaacatcatgatttccccaacattcctggaaaaagtcttccactggtgaggaaaatagcagctgaatactatgacaacctccctcactacaattcctggataaaagtactgtatgattttgtgatggatgatacaataagtccctactcaagaatgaagaggcaccaaaaaggagagatggtgctggagtaaatatcattagtgccaaagggattcttctccaaaactttagatgataaaattagccgggcgtggcggcacatgcctgtaatcccagctacatgggaggctgaggtgggagaattgcttgaacccaggaggcggaggcagaggctgcagtgacccaagattgtgccactgcactccaccctgggcaacagagcaagaccccatcntcgagagatnagatgagatatatataaaaaataaaaagctatttctagtttatttcactataaagttttgctttattaaaaagctaataaacagctattaatcacaaaaaaaaaaaaaaaaaaaaaaaa

SEQ ID NO: 35 - protein, 323 aa, Homo sapiens
MGNSASRSDFEWVYTDQPHTQRRKEILAKYPAIKALMRPDPRLKWAVLVLVLVQMLACWLVRGLAWRWLLFWAYAFGGCVNHSLTLAIHDISHNAAFGTGRAARNRWLAVFANLPVGVPYAASFKKYHVDHHRYLGGDGLDVDVPTRLEGWFFCTPARKLLWLVLQPFFYSLRPLCVHPKAVTRMEVLNTLVQLAADLAIFALWGLKPVVYLLASSFLGLGLHPISGHFVAEHYMFLKGHETYSYYGPLNWITFNVGYHVEHHDFPSIPGYNLPLVRKIAPEYYDHLPQHHSWVKVLWDFVFEDSLGPYARVKRVYRLAKDGL

SEQ ID NO: 36 - DNA, 1411 bp, Homo sapiens
aatcagagctggttccgcgccgcggccgccgcgacaggtgcagcagagccgagccggccgcgctccgaacggcgcctcccgccccaccatgggcaacagcgcgagccgcagcgacttcgagtgggtctacaccgaccagccgcacacgcagcggcgcaaggagatactggccaagtacccggccatcaaggccctgatgcggccagacccgcgcctcaagtgggcggtgctggtgctggtgctggtgcagatgctggcctgctggctggtgcgcgggctggcctggcgctggctgctgttctgggcctacgcctttggtggctgcgtgaaccactcgctgacgctggccatccacgacatctcgcacaacgcggccttcggcacgggccgtgcggcacgcaaccgctggctggccgtgttcgccaacctgcccgtgggtgtgccctacgccgcctccttcaagaagtaccacgtggaccaccaccgctacctgggcggcgacgggctggacgtggacgtgcccacgcgtctggagggctggttcttctgcacacccgcccgcaagctgctctggctggtgctgcagcccttcttctactcactacggccgctctgcgtccaccccaaggccgtgacccgcatggaggtgctcaacacgctggtgcagctggcggccgacctggccatctttgccctttgggggctcaagcccgtggtctacctgctggccagctccttcctgggcctgggcctgcaccccatctcgggccacttcgtggccgagcactacatgttcctcaagggccacgagacctactcctactatgggcctctcaactggatcaccttcaatgtgggctaccacgtggagcaccacgacttccccagcatcccgggctacaacctgccgctggtgcggaagatcgcgcccgagtactacgaccacctgccgcagcaccactcctgggtgaaggtgctctgggattttgtgtttgaggactccctggggccctatgccagggtgaagcgggtgtacaggctggcaaaagatggtctgtgagcccgggctgcctcctggtggtggccattgtcccccatcggcccctcagccttgcaccccagcactgagaagctacatttccttcctgtgctctggactgctgcccttgtccccgaggagtgtcccgcgcagccacacctggcaacagcagtgtgggctgcagggctccgtctgcacgtggacttgccctggaccttgagtgtggccctccctttctgggcctccccaggtgaggcctggccctgccccaccatgacctgggtgctctgagcccacggttcccacggagctgacttctccggggtgcctgtgccctacattaaacccggcgtttgtttcacagccaaaaaa